SEQUENCE LISTING (165 sequences)

SEQ ID NO: 1 - protein, 23 aa, Artificial sequence
  Synthetic
FALALKALKKALKKLKKALKKAL

SEQ ID NO: 2 - protein, 23 aa, Artificial Sequence
  Synthetic
FALALKALKKALKKLKKALKKAL

SEQ ID NO: 3 - protein, 38 aa, Artificial sequence
  Synthetic
MPKWKVFKKIEKVGRNIRNGIVKAGPAIAVLGEAKALG

SEQ ID NO: 4 - protein, 23 aa, Artificial sequence
  Synthetic
FAKKLAKKLKKLAKKLAKLALAL

SEQ ID NO: 5 - protein, 38 aa, Artificial sequence; identical to SEQ ID NO: 3
  Synthetic

SEQ ID NO: 6 - protein, 23 aa, Artificial sequence
  Synthetic
FAKKLAKKLKKLAKKLAKLALAL

SEQ ID NO: 7 - protein, 23 aa, Artificial sequence
  Synthetic
GIGKFLHSAKKFGKAFVGGIMNS

SEQ ID NO: 8 - protein, 23 aa, Artificial sequence
  Synthetic
FALAAKALKKLAKKLKKLAKKAL

SEQ ID NO: 9 - protein, 23 aa, Artificial sequence
  Synthetic
FALALKALKKLLKKLKKLAKKAL

SEQ ID NO: 10 - protein, 23 aa, Artificial sequence
  Synthetic
FALALKALKKLAKKLKKLAKKAL

SEQ ID NO: 11 - protein, 21 aa, Artificial sequence
  Synthetic
FALAKLAKKAKAKLKKALKAL

SEQ ID NO: 12 - protein, 19 aa, Artificial sequence
  Synthetic
FALALKALKKLKKALKKAL

SEQ ID NO: 13 - protein, 19 aa, Artificial sequence
  Synthetic
FALALKALKKLKKALKKAL

SEQ ID NO: 14 - protein, 19 aa, Artificial sequence
  Synthetic
FAKKLAKKLKKLAKLALAL

SEQ ID NO: 15 - protein, 23 aa, Artificial sequence
  Synthetic
VALALKALKKALKKLKKALKKAL

SEQ ID NO: 16 - protein, 16 aa, Artificial sequence
  Synthetic
FALALKKALKALKKAL

SEQ ID NO: 17 - protein, 17 aa, Artificial sequence
  Synthetic
FAKKLAKLAKKLAKLAL

SEQ ID NO: 18 - protein, 19 aa, Artificial sequence
  Synthetic
FAKKLAKLAKKLAKLALAL

SEQ ID NO: 19 - protein, 23 aa, Artificial sequence
  Synthetic
FALALKALKKALXXLKKALKKAL

SEQ ID NO: 20 - protein, 15 aa, Artificial sequence
  Synthetic
FAKKLAKLAKKLLAL

SEQ ID NO: 21 - protein, 15 aa, Artificial sequence
  Synthetic
FAKKLAKLAKKALAL

SEQ ID NO: 22 - protein, 15 aa, Artificial sequence
  Synthetic
FALAKKALKKAKKAL

SEQ ID NO: 23 - protein, 19 aa, Artificial sequence
  Synthetic
FAKKLAKKLKKLAKLALAK

SEQ ID NO: 24 - protein, 22 aa, Artificial sequence
  Synthetic
GIGKFLKKAKKFGKAFVKILKK

SEQ ID NO: 25 - protein, 13 aa, Artificial sequence
  Synthetic
FAKLLAKLAKKLL

SEQ ID NO: 26 - protein, 15 aa, Artificial sequence
  Synthetic
FAKKLAKLALKLAKL

SEQ ID NO: 27 - protein, 14 aa, Artificial sequence
  Synthetic
FAKKLAKKLAKLAL

SEQ ID NO: 28 - protein, 15 aa, Artificial sequence
  Synthetic
FAKKLKKLAKLAKKL

SEQ ID NO: 29 - protein, 12 aa, Artificial sequence
  Synthetic
FAKKALKALKKL

SEQ ID NO: 30 - protein, 13 aa, Artificial sequence
  Synthetic
VAKLLAKLAKKLL

SEQ ID NO: 31 - protein, 12 aa, Artificial sequence
  Synthetic
FAKLLAKLAKKL

SEQ ID NO: 32 - protein, 17 aa, Artificial sequence
  Synthetic
VAKKLAKLAKKLAKLAL

SEQ ID NO: 33 - protein, 15 aa, Artificial sequence
  Synthetic
KWKLFKKIGAVLKVL

SEQ ID NO: 34 - protein, 13 aa, Artificial sequence
  Synthetic
FAKLLAKLAKKAL

SEQ ID NO: 35 - protein, 13 aa, Artificial sequence
  Synthetic
FAKLLAKALKKLL

SEQ ID NO: 36 - protein, 13 aa, Artificial sequence
  Synthetic
FAKLLKLAAKKLL

SEQ ID NO: 37 - protein, 10 aa, Artificial sequence
  Synthetic
FAKLLAKKLL

SEQ ID NO: 38 - protein, 10 aa, Artificial sequence
  Synthetic
FAKKLAKALL

SEQ ID NO: 39 - protein, 10 aa, Artificial sequence
  Synthetic
FAKKLAKKLL

SEQ ID NO: 40 - protein, 9 aa, Artificial sequence
  Synthetic
FAKLAKKLL

SEQ ID NO: 41 - protein, 17 aa, Artificial sequence
  Synthetic
FAKKLAKLAKKLAKLAL

SEQ ID NO: 42 - protein, 13 aa, Artificial sequence
  Synthetic
ILPWKWPWWPWRR

SEQ ID NO: 43 - protein, 15 aa, Artificial sequence
  Synthetic
FAKALKALLKALKAL

SEQ ID NO: 44 - protein, 13 aa, Artificial sequence
  Synthetic
FAKLLAKLAKAKL

SEQ ID NO: 45 - protein, 13 aa, Artificial sequence
  Synthetic
FAKLLAKLAKLKL

SEQ ID NO: 46 - protein, 22 aa, Artificial sequence
  Synthetic
FAKKLAKKLKKLAKKLAKKWKL

SEQ ID NO: 47 - protein, 18 aa, Artificial sequence
  Synthetic
FAKKLAKKLKKLAKKLAK

SEQ ID NO: 48 - protein, 22 aa, Artificial sequence
  Synthetic
FAKKLAKKLKKLAKKLAKKWKL

SEQ ID NO: 49 - protein, 23 aa, Artificial sequence
  Synthetic
KWKLFKKKTKLFKKFAKKLAKKL

SEQ ID NO: 50 - protein, 13 aa, Artificial sequence
  Synthetic
FAKKLAKKLAKAL

SEQ ID NO: 51 - protein, 13 aa, Artificial sequence
  Synthetic
FAKKLAKKLAKLL

SEQ ID NO: 52 - protein, 14 aa, Artificial sequence
  Synthetic
FAKKLAKKLAKAAL

SEQ ID NO: 53 - protein, 15 aa, Artificial sequence
  Synthetic
FAKKLAKKAKLAKKL

SEQ ID NO: 54 - protein, 12 aa, Artificial sequence
  Synthetic
FAKKLKKLAKKL

SEQ ID NO: 55 - protein, 23 aa, Artificial sequence
  Synthetic
KTKLFKKFAKKLAKKLKKLAKKL

SEQ ID NO: 56 - protein, 23 aa, Artificial sequence
  Synthetic
KWKLFKKKTKLFKKFAKKLAKKL

SEQ ID NO: 57 - protein, 13 aa, Artificial sequence
  Synthetic
ILPWKWPWWPWRR

SEQ ID NO: 58 - protein, 13 aa, Artificial sequence
  Synthetic
FAKALAKLAKKLL

SEQ ID NO: 59 - protein, 13 aa, Artificial sequence
  Synthetic
FAKLLAKLAKKAA

SEQ ID NO: 60 - protein, 13 aa, Artificial sequence
  Synthetic
FAKLLALALKLKL

SEQ ID NO: 61 - protein, 13 aa, Artificial sequence
  Synthetic
FAKLLAKLAKAKA

SEQ ID NO: 62 - protein, 13 aa, Artificial sequence
  Synthetic
FAKLLAKLAKAKG

SEQ ID NO: 63 - protein, 31 aa, Artificial sequence
  Synthetic
FAKKLAKKLKKLAKKLAKLALALKALALKAL

SEQ ID NO: 64 - protein, 23 aa, Artificial sequence
  Synthetic
FAKKLAKKLKKLAKKLIGAVLKV

SEQ ID NO: 65 - protein, 13 aa, Artificial sequence
  Synthetic
FAKLLAKALKLKL

SEQ ID NO: 66 - protein, 13 aa, Artificial sequence
  Synthetic
FAKLLAKALKKAL

SEQ ID NO: 67 - protein, 12 aa, Artificial sequence
  Synthetic
FAKLLAKALKKL

SEQ ID NO: 68 - protein, 20 aa, Artificial sequence
  Synthetic
KWKLFKKALKKLKKALKKAL

SEQ ID NO: 69 - protein, 23 aa, Artificial sequence
  Synthetic
KIAKVALAKLGIGAVLKVLTTGL

SEQ ID NO: 70 - protein, 12 aa, Artificial sequence
  Synthetic
FAKKLAKLAKKL

SEQ ID NO: 71 - protein, 19 aa, Artificial sequence
  Synthetic
MPKEKVFLKIEKMGRNIRN

SEQ ID NO: 72 - protein, 26 aa, Artificial sequence
  Synthetic
GIGAVLKVLTTGLPALISWIKRKRQQ

SEQ ID NO: 73 - protein, 16 aa, Artificial sequence
  Synthetic
FAKKLAKLAKKLAKAL

SEQ ID NO: 74 - protein, 12 aa, Artificial sequence
  Synthetic
FAKKLLAKALKL

SEQ ID NO: 75 - protein, 13 aa, Artificial sequence
  Synthetic
FAKFLAKFLKKAL

SEQ ID NO: 76 - protein, 13 aa, Artificial sequence
  Synthetic
FAKLLFKALKKAL

SEQ ID NO: 77 - protein, 13 aa, Artificial sequence
  Synthetic
FAKLLAKFLKKAL

SEQ ID NO: 78 - protein, 13 aa, Artificial sequence
  Synthetic
FAKLLAKAFKKAL

SEQ ID NO: 79 - protein, 13 aa, Artificial sequence
  Synthetic
FAKLFAKAFKKAL

SEQ ID NO: 80 - protein, 13 aa, Artificial sequence
  Synthetic
FAKLLAKALKKFL

SEQ ID NO: 81 - protein, 14 aa, Artificial sequence
  Synthetic
FAKLLAKALKKFAL

SEQ ID NO: 82 - protein, 14 aa, Artificial sequence
  Synthetic
FAKLLAKLAKKFAL

SEQ ID NO: 83 - protein, 14 aa, Artificial sequence
  Synthetic
FAKLFAKLAKKFAL

SEQ ID NO: 84 - protein, 13 aa, Artificial sequence
  Synthetic
FKLAFKLAKKAFL

SEQ ID NO: 85 - protein, 10 aa, Artificial sequence
  Synthetic
FAKLLAKLAK

SEQ ID NO: 86 - protein, 13 aa, Artificial sequence
  Synthetic
FAKLLAKLAKKVL

SEQ ID NO: 87 - protein, 13 aa, Artificial sequence
  Synthetic
FAKLLAKLAKKIL

SEQ ID NO: 88 - protein, 13 aa, Artificial sequence
  Synthetic
FAKLLAKLAKKEL

SEQ ID NO: 89 - protein, 13 aa, Artificial sequence
  Synthetic
FAKLLAKLAKKSL

SEQ ID NO: 90 - protein, 5 aa, Artificial sequence
  Synthetic
FAKLA

SEQ ID NO: 91 - protein, 5 aa, Artificial sequence
  Synthetic
FAKLF

SEQ ID NO: 92 - protein, 5 aa, Artificial sequence
  Synthetic
KAKLF

SEQ ID NO: 93 - protein, 5 aa, Artificial sequence
  Synthetic
KWKLF

SEQ ID NO: 94 - protein, 13 aa, Artificial sequence
  Synthetic
FGKGIGKVGKKLL

SEQ ID NO: 95 - protein, 15 aa, Artificial sequence
  Synthetic
FAFGKGIGKVGKKLL

SEQ ID NO: 96 - protein, 22 aa, Artificial sequence
  Synthetic
FAKAIAKIAFGKGIGKVGKKLL

SEQ ID NO: 97 - protein, 22 aa, Artificial sequence
  Synthetic
FAKLWAKLAFGKGIGKVGKKLL

SEQ ID NO: 98 - protein, 12 aa, Artificial sequence
  Synthetic
FAKLWAKLAKKL

SEQ ID NO: 99 - protein, 13 aa, Artificial sequence
  Synthetic
FAKGVGKVGKKAL

SEQ ID NO: 100 - protein, 15 aa, Artificial sequence
  Synthetic
FAFGKGIGKIGKKGL

SEQ ID NO: 101 - protein, 16 aa, Artificial sequence
  Synthetic
FAKIIAKIAKIAKKIL

SEQ ID NO: 102 - protein, 15 aa, Artificial sequence
  Synthetic
FAFAKIIAKIAKKII

SEQ ID NO: 103 - protein, 7 aa, Artificial sequence
  Synthetic
FALALKA

SEQ ID NO: 104 - protein, 12 aa, Artificial sequence
  Synthetic
KWKLAKKALALL

SEQ ID NO: 105 - protein, 12 aa, Artificial sequence
  Synthetic
FAKIIAKIAKKI

SEQ ID NO: 106 - protein, 12 aa, Artificial sequence
  Synthetic
FALALKALKKAL

SEQ ID NO: 107 - protein, 8 aa, Artificial sequence
  Synthetic
FALKALKK

SEQ ID NO: 108 - protein, 13 aa, Artificial sequence
  Synthetic
KYKKALKKLAKLL

SEQ ID NO: 109 - protein, 17 aa, Artificial sequence
  Synthetic
FKRLAKIKVLRLAKIKR

SEQ ID NO: 110 - protein, 13 aa, Artificial sequence
  Synthetic
FAKLAKKALAKLL

SEQ ID NO: 111 - protein, 13 aa, Artificial sequence
  Synthetic
KAKLAKKALAKLL

SEQ ID NO: 112 - protein, 17 aa, Artificial sequence
  Synthetic
KLALKLALKALKAAKLA

SEQ ID NO: 113 - protein, 11 aa, Artificial sequence
  Synthetic
FAKLLAKLAKK

SEQ ID NO: 114 - protein, 13 aa, Artificial sequence
  Synthetic
FAKLLAKLAKKGL

SEQ ID NO: 115 - protein, 1 aa, Artificial sequence
  Synthetic
M

SEQ ID NO: 116 - protein, 13 aa, Artificial sequence
  Synthetic
VAKLLAKLAKKVL

SEQ ID NO: 117 - protein, 13 aa, Artificial sequence
  Synthetic
YAKLLAKLAKKAL

SEQ ID NO: 118 - protein, 17 aa, Artificial sequence
  Synthetic
KLLKLLLKLYKKLLKLL

SEQ ID NO: 119 - protein, 26 aa, Artificial sequence
  Synthetic
FAVGLRAIKRALKKLRRGVRKVAKDL

SEQ ID NO: 120 - protein, 16 aa, Artificial sequence
  Synthetic
KLAKKLAKLAKLAKAL

SEQ ID NO: 121 - protein, 16 aa, Artificial sequence
  Synthetic
KLAKKLAKLAKLAKAL

SEQ ID NO: 122 - protein, 9 aa, Artificial sequence
  Synthetic
KWKKLAKKW

SEQ ID NO: 123 - protein, 9 aa, Artificial sequence
  Synthetic
KWKKLAKKW

SEQ ID NO: 124 - protein, 17 aa, Artificial sequence
  Synthetic
KLWKKWAKKWLKLWKAW

SEQ ID NO: 125 - protein, 16 aa, Artificial sequence
  Synthetic
KLWKKWAKKWLKLWKA

SEQ ID NO: 126 - protein, 11 aa, Artificial sequence
  Synthetic
FALALKALKKL

SEQ ID NO: 127 - protein, 11 aa, Artificial sequence
  Synthetic
FALAKALKKAL

SEQ ID NO: 128 - protein, 12 aa, Artificial sequence
  Synthetic
FALALKLAKKAL

SEQ ID NO: 129 - protein, 6 aa, Artificial sequence
  Synthetic
FALLKL

SEQ ID NO: 130 - protein, 10 aa, Artificial sequence
  Synthetic
FALALKALKK

SEQ ID NO: 131 - protein, 10 aa, Artificial sequence
  Synthetic
FALKALKKAL

SEQ ID NO: 132 - protein, 11 aa, Artificial sequence
  Synthetic
FALLKALKKAL

SEQ ID NO: 133 - protein, 4 aa, Artificial sequence
  Synthetic
KWKK

SEQ ID NO: 134 - protein, 5 aa, Artificial sequence
  Synthetic
KWKKL

SEQ ID NO: 135 - protein, 9 aa, Artificial sequence
  Synthetic
KFKKLAKKF

SEQ ID NO: 136 - protein, 9 aa, Artificial sequence
  Synthetic
KFKKLAKKW

SEQ ID NO: 137 - protein, 11 aa, Artificial sequence
  Synthetic
FALALKALKKA

SEQ ID NO: 138 - protein, 12 aa, Artificial sequence
  Synthetic
FALLKALLKKAL

SEQ ID NO: 139 - protein, 11 aa, Artificial sequence
  Synthetic
FALALKLAKKL

SEQ ID NO: 140 - protein, 11 aa, Artificial sequence
  Synthetic
LKKLAKLALAF

SEQ ID NO: 141 - protein, 11 aa, Artificial sequence
  Synthetic
VALALKALKKL

SEQ ID NO: 142 - protein, 10 aa, Artificial sequence
  Synthetic
FALALKLKKL

SEQ ID NO: 143 - protein, 10 aa, Artificial sequence
  Synthetic
FALALKAKKL

SEQ ID NO: 144 - protein, 4 aa, Artificial sequence
  Synthetic
FALA

SEQ ID NO: 145 - protein, 5 aa, Artificial sequence
  Synthetic
WALAL

SEQ ID NO: 146 - protein, 23 aa, Artificial sequence
  Synthetic
GIGKFLHAAKKFAKAFVAEIMNS

SEQ ID NO: 147 - protein, 23 aa, Artificial sequence
  Synthetic
FAKKFAKKFKKFAKKFAKFAFAF

SEQ ID NO: 148 - protein, 10 aa, Artificial sequence
  Synthetic
KKVVFKVKFK

SEQ ID NO: 149 - protein, 10 aa, Artificial sequence
  Synthetic
FKVKFKVKVK

SEQ ID NO: 150 - protein, 38 aa, Artificial sequence
  Synthetic
LPKWKVFKKIEKVGRNIRNGIVKAGPAIAVLGEAKALG

SEQ ID NO: 151 - protein, 23 aa, Artificial sequence
  Synthetic
FAKKLAKKLKKLAKKLAKLAKKL

SEQ ID NO: 152 - protein, 15 aa, Artificial sequence
  Synthetic
VAKALKALLKALKAL

SEQ ID NO: 153 - protein, 13 aa, Artificial sequence
  Synthetic
VAKFLAKFLKKAL

SEQ ID NO: 154 - protein, 23 aa, Artificial sequence
  Synthetic
VAKKFAKKFKKFAKKFAKFAFAF

SEQ ID NO: 155 - protein, 19 aa, Artificial sequence
  Synthetic
VAKKLAKLAKKLAKLALAL

SEQ ID NO: 156 - protein, 15 aa, Artificial sequence
  Synthetic
VAKKLAKLAKKLLAL

SEQ ID NO: 157 - protein, 13 aa, Artificial sequence
  Synthetic
VAKLLAKALKKLL

SEQ ID NO: 158 - protein, 23 aa, Artificial sequence
  Synthetic
VALALKALKKALKKLKKALKKAL

SEQ ID NO: 159 - protein, 23 aa, Artificial sequence
  Synthetic
VALALKALKKALKKLKKALKKAL

SEQ ID NO: 160 - protein, 23 aa, Artificial sequence
  Synthetic
VALALKALKKLAKKLKKLAKKAL

SEQ ID NO: 161 - protein, 23 aa, Artificial sequence
  Synthetic
VALALKALKKLLKKLKKLAKKAL

SEQ ID NO: 162 - protein, 23 aa, Artificial sequence
  Synthetic
FAKKLAKKLKKLAKKLAKLALAL

SEQ ID NO: 163 - protein, 30 aa, Artificial sequence
  Synthetic
FAKKLAKKLKKLAKKLAKLALALKALALKA

SEQ ID NO: 164 - protein, 18 aa, Artificial sequence
  Synthetic
FAKKLAKKLKKLAKKLAK

SEQ ID NO: 165 - protein, 13 aa, Artificial sequence
  Synthetic
FAKLLALALKKAL

(19) United States
(12) Patent Application Publication
(10) Pub. No.: US 2005/0209157 A1
(43) Pub. Date: Sep. 22, 2005

(54) Short bioactive peptides and methods for their use

(76) Inventor: Donald R. Owen, Kenner, LA (US)

Correspondence Address: HOWREY LLP, C/O IP DOCKETING DEPARTMENT, 2941 FAIRVIEW PARK DRIVE, SUITE 200, FALLS CHURCH, VA, 22042-7195, US

(21) Appl. No.: 11/136,186

(22) Filed: May 24, 2005

Related U.S. Application Data
(62) Division of application No. 10/109,171, filed on Mar. 28, 2002.
(60) Provisional application No. 60/279,505, filed on Mar. 28, 2001.

(51) Int. Cl. A61K 38/10; A61K 38/08; C07K 7/08; C07K 7/06

(52) U.S. Cl. 514/13; 514/14; 514/15; 530/326; 530/327; 530/328; 530/329

(57) ABSTRACT

Short bioactive peptides containing phenylalanine, leucine, alanine, and lysine residues are disclosed. The peptides can be used in antibacterial, antifungal, anticancer, and other biological applications.

### CROSS REFERENCE TO RELATED APPLICATIONS

This is a divisional of co-pending U.S. patent application Ser. No. 10/109,171, filed Mar. 28, 2002; which claims the benefit of U.S. Provisional Patent Application Ser. No. 60/279,505 filed Mar. 28, 2001. Each of the foregoing applications is herein incorporated by reference.

### FIELD OF THE INVENTION

The invention relates to short length peptides containing phenylalanine, leucine, alanine, and lysine amino acid residues (F, L, A, and K; “FLAK peptides”) in their primary sequence. In particular, FLAK peptides having desirable antimicrobial, antifungal, anticancer, and other biological activities are disclosed.

### BACKGROUND OF THE INVENTION

Various bioactive peptides have been reported in both the scientific literature and in issued patents. Peptides historically have been isolated from natural sources, and have recently been the subject of structure-function relationship studies. Additionally, natural peptides have served as starting points for the design of synthetic peptide analogs.

A review of peptide antibiotics was published by R. E. W. Hancock in 1997 (Lancet 349: 418-422). The structure, function, and clinical applications of various classes of peptides were discussed. An additional review of cationic peptide antibiotics was published in 1998 (Hancock, R. E. W. and Lehrer, R. Trends Biotechnol. 16: 82-88). The peptides are typically cationic amphipathic molecules of 12 to 45 amino acids in length. The peptides permeabilize cell membranes leading to the control of microbial agents. The clinical potential of host defense cationic peptides was discussed by R. E. W. Hancock in 1999 (Drugs 57(4): 469-473; Antimicrobial Agents and Chemotherapy 43(6): 1317-1323). The antibacterial, antifungal, antiviral, anticancer, and wound healing properties of the class of peptides are discussed.

Reviews of the structural features of helical antimicrobial peptides, and their presumed mechanisms of action have been published (see, for example, Dathe, M. and Wieprecht, T. Biochimica et Biophysica Acta 1462: 71-87 (1999); Epand, R. M. and Vogel H. J. Biochimica et Biophysica Acta 1462: 11-28 (1999)). Structural parameters believed to be capable of modulating activity and selectivity include helicity, hydrophobic moment, hydrophobicity, angle subtended by the hydrophilic/hydrophobic helix surfaces, and charge.

A wide array of naturally occurring alpha helical peptides have been reported. The following are representative of the many references in the field.

Cecropins are a family of α-helical peptides isolated from insects. Cecropins are known for their antibacterial properties, as described in U.S. Pat. Nos. 4,355,104 and 4,520,016. The cecropins were generally found to have activity against gram-negative bacteria, but not against all gram-negative bacteria. Cecropins were found not to have activity against eucaryotic cells (Andreu, et al., Biochemistry 24: 163-188 (1985); Boman, et al., Developmental and Comparative Immunol. 9: 551-558 (1985); Steiner et al., Nature 292: 246-248 (1981)). Cecropins from Drosophila and Hyalphora were presented as having activity against various strains of fungi (Ekengren, S. and Hultmark, D., Insect Biochem. and Molec. Biol. 29: 965-972 (1999)). Cecropin A from mosquito Aedes aegypti is reportedly different from most insect cecropins in that it lacks tryptophan and C-terminal amidation (Lowenberger, C. et al., J. Biol. Chem. 274(29): 20092-20097 (1999)).

Frogs from the genus Rana produce a wide array of antimicrobial peptides in their skin (Goraya, J. et al., Eur. J. Biochem. 267: 894-900 (2000)). Peptides as short as 13 amino acids were reported, and were grouped into structural families. The sequences showed little or no sequence identity to peptides isolated from frogs of other genera, such as the magainin and dermaseptin peptides.

U.S. Pat. No. 5,962,410 disclosed the inhibition of eucaryotic pathogens, and the stimulation of lymphocytes and fibroblasts with lytic peptides such as cecropins and sarcotoxins. Various peptides presented include Cecropin B, Cecropin SB-37, Cecropin A, Cecropin D, Shiva-1, Lepidopteran, Sarcotoxin 1A, Sarcotoxin 1B, and Sarcotoxin 1C.

Transgenic mice producing the Shiva-1 cecropin class lytic peptide were reported by Reed, W. A. et al., Transgenic Res. 6: 337-347 (1997). Infection of the transgenic mice with a Brucella abortus challenge resulted in a reduction of the number of bacteria relative to infection of non-transgenic mice.

Magainin is an α-helical 23 amino acid peptide isolated from the skin of the African frog Xenopus laevis (Zasloff, M. Proc. Natl. Acad. Sci. USA. 84: 5449-5453 (1987).

Cathelin associated α-helical peptides of 23 to 38 amino acids are found in the blood cells of sheep, humans, cattle, pigs, mice, and rabbits (Zanetti, M. et al., FEBS Lett. 374: 1-5 (1995)).

The antimicrobial activities of buforin II, cecropin P1, indolicidin, magainin II, nisin, and ranalexin were reported by Giacomette, A. et al. (Peptides 20: 1265-1273 (1999)). The peptides showed variable activities against bacteria and yeast.

Various synthetic peptides have been prepared and assayed both in vitro and in vivo.

U.S. Pat. No. 5,861,478 disclosed synthetic lytic peptides of about 20 to 40 amino acids which adopt an α-helical conformation. The peptides are effective in the treatment of microbial infections, wounds, and cancer. The peptides disclosed include cecropin B, SB-37*, LSB-37, SB-37, Shiva 1 and 10-12, β-fibrin signal peptide, Manitou 1-2, Hecate 1-3, Anubis 1-5 and 8, and Vishnu 1-3 and 8.

Hecate was described as a synthetic peptide analog of melittin by Baghian, A. et al. (Peptides 18(2): 177-183 (1997)). The peptides differ in their charge distribution, but not in their amphipathic alpha helical conformation. Hecate inhibited herpes simplex virus (HSV-1) while not adversely affecting cell growth and protein synthesis.

Synthetic peptides D2A21, D4E1, D2A22, D5C, D5C1, D4E, and D4B were described in Schwab, U. et al., Antimicrob. Agents and Chemotherapy 43(6): 1435-1440 (1999). Activities against various bacterial strains were presented.

Hybrid peptides made of cecropin and melittin peptides were reportedly prepared and assayed by Juvvadi, P. et al. (J. Peptide Res. 53: 244-251 (1999)). Hybrids were synthesized to investigate the effects of sequence, amide bond direction (helix dipole), charge, amphipathicity, and hydrophobicity on channel forming ability and on antibacterial activity. Sequence and amide bond direction were suggested to be important structural requirements for the activity of the hybrids.

A 26 amino acid insect cecropin—bee melittin hybrid, and analogs thereof, were described in a study of salt resistance (Friedrich, C. et al., Antimicrobial Agents and Chemotherapy 43(7): 1542-1548 (1999)). A tryptophan residue in the second position was found to be critical for activity. Modest changes in sequence were found to lead to substantial changes in the properties of the peptides.

The effects of proline residues on the antibacterial properties of α-helical peptides has been published (Zhang, L. et al., Biochem. 38: 8102-8111 (1999)). The addition of prolines was reported to change the membrane insertion properties, and the replacement of a single proline may change an antimicrobial peptide into a toxin.

A series of peptides having between 18 and 30 amino acids were prepared in order to test the effects of changes in sequence and charge on antibacterial properties (Scott, M. G., et al., Infect. Immun. 67(4): 2005-2009 (1999)). No significant correlation was found between length, charge, or hydrophobicity and the antimicrobial activity of the peptides. A general trend was found that shorter peptides were less active than longer peptides, although the authors expressed that this effect would probably be sequence dependent.

“Modellins”, a group of synthetic peptides were prepared and assayed to compare sequence and structure relationships (Bessalle, R. et al. J. Med. Chem. 36: 1203-1209 (1993)). Peptides of 16 and 17 amino acids having hydrophobic and hydrophilic opposite faces were highly hemolytic and antibacterial. Smaller peptides tended to have lower biological activities.

A cecropin-melittin hybrid peptide and an amidated flounder peptide were found to protect salmon from Vibrio anguillarum infections in vivo (Jia, X. et al., Appl. Environ. Microbiol. 66(5): 1928-1932 (2000)). Osmotic pumps were used to deliver a continuous dose of either peptide to the fish.

Amphipathic peptides have been reported as being capable of enhancing wound healing and stimulating fibroblast and keratinocyte growth in vivo (U.S. Pat. Nos. 6,001,805 and 5,561,107). Transgenic plants have been reportedly prepared expressing lytic peptides as a fusion protein with ubiquitin (U.S. Pat. No. 6,084,156). Methylated lysine rich lytic peptides were reportedly prepared, displaying improved proteolytic resistance (U.S. Pat. No. 5,717,064).

While a number of natural and synthetic peptides exist, there exists a need for improved bioactive peptides and methods for their use.

### SUMMARY OF THE INVENTION

Short (i.e. no more than 23 amino acids in length) peptides containing phenylalanine, leucine, alanine, and lysine amino acid residues in their primary sequence are disclosed. The peptides display desirable antibacterial, antifungal, anticancer biological activities, and also cause stimulation and proliferation of human fibroblasts and lymphocytes.

### DESCRIPTION OF THE SEQUENCE LISTINGS

The following sequence listings form part of the present specification and are included to further demonstrate certain aspects of the present invention. The invention may be better understood by reference to one or more of these sequences in combination with the detailed description of specific embodiments presented herein.

TABLE 1

SEQ

| | |
|---|---|
| ID | P- |

| | | | |
|---|---|---|---|
| NO: | Name | No. | Primary sequence |

| | | | |
|---|---|---|---|
| 1 | Hecate AC #1010 | 1 | FALALKALKKALKKLKKALKKAL-COOH |
| 2 | Hecate AM | 2 | FALALKALKKALKKLKKALKKAL-NH2 |
| 3 | SB-37 AC #1018 | 5 | MPKWKVFKKIEKVGRNIRNGIVKAGPAIAVLGEAKALG-COOH |
| 4 | Shiva 10 AM | 11 | FAKKLAKKLKKLAKKLAKLALAL-NH2 |
| 5 | SB-37 AM | 12 | MPKWKVFKKIEKVGRNIRNGIVKAGPAIAVLGEAKALG-NH2 |
| 6 | Shiva 10 AC #1015 | 13 | FAKKLAKKLKKLAKKLAKLALAL-COOH |
| 7 | Magainin 2 | 16 | GIGKFLHSAKKFGKAFVGGIMNS-NH2 |
| 8 | FLAK01 AM | 23 | FALAAKALKKLAKKLKKLAKKAL-NH2 |
| 9 | FLAK03 AM | 24 | FALALKALKKLLKKLKKLAKKAL-NH2 |
| 10 | FLAK04 AM | 25 | FALALKALKKLAKKLKKLAKKAL-NH2 |
| 11 | FLAK05 AM | 26 | FALAKLAKKAKAKLKKALKAL-NH2 |
| 12 | FLAK06 AM | 27 | FALALKALKKLKKALKKAL-NH2 |
| 13 | FLAK06 AC | 28 | FALALKALKKLKKALKKAL-COOH |
| 14 | FLAK06 R-AC | 29 | FAKKLAKKLKKLAKLALAL-COOH |
| 15 | KAL V | 30 | VALALKALKKALKKLKKALKKAL-NH2 |
| 16 | FLAK 17 AM | 34 | FALALKKALKALKKAL-NH2 |
| 17 | FLAK 26 AM | 35 | FAKKLAKLAKKLAKLAL-NH2 |
| 18 | FLAK 25 AM | 36 | FAKKLAKLAKKLAKLALAL-NH2 |
| 19 | Hecate 2DAc | 37 | FALALKALKKAL-(D)-K-(D)-KLKKALKKAL-COOH |
| 20 | FLAK43 AM | 38 | FAKKLAKLAKKLLAL-NH2 |
| 21 | FLAK44 AM | 39 | FAKKLAKLAKKALAL-NH2 |
| 22 | FLAK62 AM | 40 | FALAKKALKKAKKAL-NH2 |
| 23 | FLAK 06R-AM | 41 | FAKKLAKKLKKLAKLALAK-NH2 |
| 24 | MSI-78 AM | 42 | GIGKFLKKAKKFGKAFVKILKK-NH2 |
| 25 | FLAK50 | 43 | FAKLLAKLAKKLL-NH2 |
| 26 | FLAK51 | 44 | FAKKLAKLALKLAKL-NH2 |
| 27 | FLAK57 | 45 | FAKKLAKKLAKLAL-NH2 |
| 28 | FLAK71 | 46 | FAKKLKKLAKLAKKL-NH2 |
| 29 | FLAK77 | 47 | FAKKALKALKKL-NH2 |
| 30 | FLAK50V | 48 | VAKLLAKLAKKLL-NH2 |
| 31 | FLAK50F | 49 | FAKLLAKLAKKL-NH2 |
| 32 | FLAK26V AM | 50 | VAKKLAKLAKKLAKLAL-NH2 |
| 33 | CAME-15 | 53 | KWKLFKKIGAVLKVL-NH2 |
| 34 | FLAK50C | 54 | FAKLLAKLAKKAL-NH2 |
| 35 | FLAK50D | 55 | FAKLLAKALKKLL-NH2 |
| 36 | FLAK50E | 56 | FAKLLKLAAKKLL-NH2 |
| 37 | FLAK80 | 57 | FAKLLAKKLL-NH2 |
| 38 | FLAK81 | 58 | FAKKLAKALL-NH2 |
| 39 | FLAK82 | 59 | FAKKLAKKLL-NH2 |
| 40 | FLAK83M | 60 | FAKLAKKLL-NH2 |
| 41 | FLAK 26 Ac | 61 | FAKKLAKLAKKLAKLAL-COOH |
| 42 | Indolicidin | 63 | ILPWKWPWWPWRR-NH2 |
| 43 | FLAK 17C | 64 | FAKALKALLKALKAL-NH2 |
| 44 | FLAK 50H | 65 | FAKLLAKLAKAKL-NH2 |
| 45 | FLAK 50G | 66 | FAKLLAKLAKLKL-NH2 |
| 46 | Shiva Deriv | 70 | FAKKLAKKLKKLAKKLAKKWKL-NH2 |
| | P69 + KWKL | | |
| 47 | Shiva 10 (1-18 AC) | 71 | FAKKLAKKLKKLAKKLAK-COOH |
| 48 | Shiva 10 peptide | 72 | FAKKLAKKLKKLAKKLAKKWKL-COOH |
| | 71 + KWKL | | |
| 49 | CA(1-7)Shiva10 | 73 | KWKLFKKKTKLFKKFAKKLAKKL-NH2 |
| | (1-16) | | |
| 50 | FLAK 54 | 74 | FAKKLAKKLAKAL-NH2 |
| 51 | FLAK 56 | 75 | FAKKLAKKLAKLL-NH2 |
| 52 | FLAK 58 | 76 | FAKKLAKKLAKAAL-NH2 |
| 53 | FLAK 72 | 77 | FAKKLAKKAKLAKKL-NH2 |
| 54 | FLAK 75 | 79 | FAKKLKKLAKKL-NH2 |
| 55 | Shiva 10 (1-16) Ac | 80 | KTKLFKKFAKKLAKKLKKLAKKL-COOH |
| 56 | CA(1-7)Shiva10 | 81 | KWKLFKKKTKLFKKFAKKLAKKL-COOH |
| | (1-16)-COOH | | |
| 57 | Indolocidin-ac | 91 | ILPWKWPWWPWRR-COOH |
| 58 | FLAK50B | 92 | FAKALAKLAKKLL-NH2 |
| 59 | FLAK50J | 93 | FAKLLAKLAKKAA-NH2 |
| 60 | FLAK50I | 94 | FAKLLALALKLKL-NH2 |
| 61 | FLAK50K | 9S | FAKLLAKLAKAKA-NH2 |
| 62 | FLAK50L | 96 | FAKLLAKLAKAKG-NH2 |
| 63 | Shiva-11 | 98 | FAKKLAKKLKKLAKKLAKLALALKALALKAL-NH2 |
| 64 | Shiva 11 | 99 | FAKKLAKKLKKLAKKLIGAVLKV-COOH |
| | [(1-16)ME(2-9]- | | |
| | COOH | | |
| 65 | FLAK 50N | 101 | FAKLLAKALKLKL-NH2 |
| 66 | FLAK 50O | 102 | FAKLLAKALKKAL-NH2 |
| 67 | FLAK 50P | 103 | FAKLLAKALKKL-NH2 |
| 68 | CA(1- | 104 | KWKLFKKALKKLKKALKKAL-NH2 |
| | &Hecate(11/23) | | |
| 69 | PYL-ME | 105 | KIAKVALAKLGIGAVLKVLTTGL-NH2 |
| 70 | FLAG26-D1 | 106 | FAKKLAKLAKKL-NH2 |
| 71 | Vishnu3 | 107 | MPKEKVFLKIEKMGRNIRN-NH2 |
| 72 | Melittin | 108 | GIGAVLKVLTTGLPALISWIKRKRQQ-NH2 |
| 73 | FLAK26-D2 | 109 | FAKKLAKLAKKLAKAL-NH2 |
| 74 | FLAG26-D3 | 110 | FAKKLLAKALKL-NH2 |
| 75 | FLAK50 Q1 | 111 | FAKFLAKFLKKAL-NH2 |
| 76 | FLAK50 Q2 | 112 | FAKLLFKALKKAL-NH2 |
| 77 | FLAK50 Q3 | 113 | FAKLLAKFLKKAL-NH2 |
| 78 | FLAK50 Q4 | 114 | FAKLLAKAFKKAL-NH2 |
| 79 | FLAK50 Q5 | 117 | FAKLFAKAFKKAL-NH2 |
| 80 | FLAK50 Q6 | 118 | FAKLLAKALKKFL-NH2 |
| 81 | FLAK50 Q7 | 119 | FAKLLAKALKKFAL-NH2 |
| 82 | FLAK50 Q8 | 120 | FAKLLAKLAKKFAL-NH2 |
| 83 | FLAK50 Q9 | 121 | FAKLFAKLAKKFAL-NH2 |
| 84 | FLAK50 Q10 | 122 | FKLAFKLAKKAFL-NH2 |
| 85 | FLAK50 T1 | 123 | FAKLLAKLAK-NH2 |
| 86 | FLAK50 T2 | 124 | FAKLLAKLAKKVL-NH2 |
| 87 | FLAK50 T3 | 125 | FAKLLAKLAKKIL-NH2 |
| 88 | FLAK50 T4 | 126 | FAKLLAKLAKKEL-NH2 |
| 89 | FLAK50 T5 | 127 | FAKLLAKLAKKSL-NH2 |
| 90 | FLAK90 | 128 | FAKLA-NH2 |
| 91 | FLAK91 | 129 | FAKLF-NH2 |
| 92 | FLAK92 | 130 | KAKLF-NH2 |
| 93 | FLAK93 | 131 | KWKLF-NH2 |
| 94 | FLAK50 Z1 | 132 | FGKGIGKVGKKLL-NH2 |
| 95 | FLAK50 Z2 | 133 | FAFGKGIGKVGKKLL-NH2 |
| 96 | FLAK50 Z3 | 134 | FAKAIAKIAFGKGIGKVGKKLL-NH2 |
| 97 | FLAK50 Z4 | 135 | FAKLWAKLAFGKGIGKVGKKLL-NH2 |
| 98 | FLAK50 Z5 | 136 | FAKLWAKLAKKL-NH2 |
| 99 | FLAK50 Z6 | 137 | FAKGVGKVGKKAL-NH2 |
| 100 | FLAK50 Z7 | 138 | FAFGKGIGKIGKKGL-NH2 |
| 101 | FLAK50 Z8 | 139 | FAKIIAKIAKIAKKIL-NH2 |
| 102 | FLAK50 Z9 | 140 | FAFAKIIAKIAKKII-NH2 |
| 103 | FLAK94 | 141 | FALALKA-NH2 |
| 104 | FLAK93B | 142 | KWKLAKKALALL-NH2 |
| 105 | FLAK50 Z10 | 143 | FAKIIAKIAKKI-NH2 |
| 106 | FLAK96 | 144 | FALALKALKKAL-NH2 |
| 107 | FLAK97 | 145 | FALKALKK-NH2 |
| 108 | FLAK98 | 146 | KYKKALKKLAKLL-NH2 |
| 109 | FKRLA | 147 | FKRLAKIKVLRLAKIKR-NH2 |
| 110 | FLAK91B | 148 | FAKLAKKALAKLL-NH2 |
| 111 | FLAK92B | 149 | KAKLAKKALAKLL-NH2 |
| 112 | FLAK99 | 150 | KLALKLALKALKAAKLA-NH2 |
| 113 | FLAK50T6 | 151 | FAKLLAKLAKK-NH2 |
| 114 | FLAK50T7 | 152 | FAKLLAKLAKKGL-NH2 |
| 115 | FLAK95 | 153 | FALKALKKLKKALKKAL-NH2 |
| 116 | FLAK50T8 | 154 | VAKLLAKLAKKVL-NH2 |
| 117 | FLAK50T9 | 155 | YAKLLAKLAKKAL-NH2 |
| 118 | FLAK100-CO2H | 156 | KLLKLLLKLYKKLLKLL-COOH |
| 119 | FAGVL | 157 | FAVGLRAIKRALKKLRRGVRKVAKDL-NH2 |
| 120 | Modelin-5 | 159 | KLAKKLAKLAKLAKAL-NH2 |
| 121 | Modelin-5-CO2H | 160 | KLAKKLAKLAKLAKAL-COOH |
| 122 | Modelin-8 | 161 | KWKKLAKKW-NH2 |
| 123 | Modelin-8-CO2H | 162 | KWKKLAKKW-COOH |
| 124 | Modelin-1 | 163 | KLWKKWAKKWLKLWKAW-NH2 |
| 125 | Modelin-1-CO2H | 164 | KLWKKWAKKWLKLWKA-COOH |
| 126 | FLAK120 | 165 | FALALKALKKL-NH2 |
| 127 | FLAK121 | 166 | FALAKALKKAL-NH2 |
| 128 | FLAK96B | 167 | FALALKLAKKAL-NH2 |
| 129 | FLAK96G | 168 | FALLKL-NH2 |
| 130 | FLAK96F | 169 | FALALKALKK-NH2 |
| 131 | FLAK96C | 170 | FALKALKKAL-NH2 |
| 132 | FLAK96D | 171 | FALLKALKKAL-NH2 |
| 133 | Modelin-8B | 172 | KWKK-NH2 |
| 134 | Modelin-8C | 173 | KWKKL-NH2 |
| 135 | Modelin-8D | 174 | KFKKLAKKF-NH2 |
| 136 | Modelin-8E | 175 | KFKKLAKKW-NH2 |
| 137 | Flak 96 | 176 | FALALKALKKA-NH2 |
| 138 | Flak 96I | 177 | FALLKALLKKAL-NH2 |
| 139 | Flak 96J | 178 | FALALKLAKKL-NH2 |
| 140 | Flak 96L | 179 | LKKLAKLALAF-NH2 |
| 141 | FLAK-120G | 180 | VALALKALKKL-NH2 |
| 142 | FLAK-120D | 181 | FALALKLKKL-NH2 |
| 143 | FLAK-120C | 182 | FALALKAKKL-NH2 |
| 144 | FLAK-120B | 183 | FALA-NH2 |
| 145 | FLAK-120F | 184 | WALAL-NH2 |
| 146 | Magainin2wisc | 300 | GIGKFLHAAKKFAKAFVAEIMNS-NH2 |
| 147 | D2A21 | 301 | FAKKFAKKFKKFAKKFAKFAFAF-NH2 |
| 148 | KSL-1 | 302 | KKVVFKVKFK-NH2 |
| 149 | KSL-7 | 303 | FKVKFKVKVK-NH2 |
| 150 | LSB-37 | 306 | LPKWKVFKKIEKVGRNIRNGIVKAGPAIAVLGEAKALG-NH2 |
| 151 | Anubis-2 | 307 | FAKKLAKKLKKLAKKLAKLAKKL-NH2 |
| 152 | FLAK17CV | 501 | VAKALKALLKALKAL-NH2 |
| 153 | FLAK50Q1V | 502 | VAKFLAKFLKKAL-NH2 |
| 154 | D2A21v | 503 | VAKKFAKKFKKFAKKFAKFAFAF-NH2 |
| 155 | FLAK25AMV | 504 | VAKKLAKLAKKLAKLALAL-NH2 |
| 156 | FLAK43AMV | 505 | VAKKLAKLAKKLLAL-NH2 |
| 157 | FLAK50DV | 506 | VAKLLAKALKKLL-NH2 |
| 158 | HECATE AMV | 507 | VALALKALKKALKKLKKALKKAL-NH2 |
| 159 | HECATE ACV | 508 | VALALKALKKALKKLKKALKKAL-COOH |
| 160 | FLAK04AMV | 509 | VALALKALKKLAKKLKKLAKKAL-NH2 |
| 161 | FLAK03AMV | 510 | VALALKALKKLLKKLKKLAKKAL-NH2 |
| 162 | D-Shiva 10 AC | 67 | (D)-FAKKLAKKLKKLAKKLAKLALAL-COOH |
| 163 | Shiva 11 AC | 100 | FAKKLAKKLKKLAKKLAKLALALKALALKA-COOH |
| 164 | Shiva 10 (1-18)AM | 69 | FAKKLAKKLKKLAKKLAK-NH2 |
| 165 | FLAK 50M | 97 | FAKLLALALKKAL-NH2 |

### DETAILED DESCRIPTION OF THE INVENTION

The invention is generally directed towards peptides having desirable biological properties, and their use. It is surprising that the peptides are efficacious due to their short length as compared to other peptides described in the art.

Peptides

One embodiment of the invention is directed towards an isolated peptide comprising phenylalanine, leucine, alanine, and lysine residues, wherein the peptide is about 5 to about 23 amino acids in length. The peptide can have a minimum length of about 5, 6, 7, 8, 9, 10, 11, 12, 13, 14, 15, 16, 17, or about 18 amino acids. The peptide can have a maximum length of about 5, 6, 7, 8, 9, 10, 11, 12, 13, 14, 15, 16, 17, 18, 19, 20, 21, 22, or about 23 amino acids. The peptide can be about 5 to about 20 amino acids in length. The peptide can consist essentially of, or consist of phenylalanine, leucine, alanine, and lysine residues. The peptide can have a percent amino acid composition of phenylalanine, leucine, alanine, and lysine residues of at least about 50%, 55%, 60%, 65%, 70%, 75%, 80%, 85%, 90%, 95%, 96%, 97%, 98%, 99%, or 100%. The peptide can generally be any of the listed SEQ ID NOS which fall within these various guidelines, and more preferably is SEQ ID NO:1, SEQ ID NO:2, SEQ ID NO:4, SEQ ID NO:5, SEQ ID NO:6, SEQ ID NO:8, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:23, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:34, SEQ ID NO:35, SEQ ID NO:36, SEQ ID NO:41, SEQ ID NO:43, SEQ ID NO:45, SEQ ID NO:46, SEQ ID NO:50, SEQ ID NO:51, SEQ ID NO:52, SEQ ID NO:55, SEQ ID NO:56, SEQ ID NO:57, SEQ ID NO:58, SEQ ID NO:59, SEQ ID NO:60, SEQ ID NO:61, SEQ ID NO:65, SEQ ID NO:66, SEQ ID NO:67, SEQ ID NO:68, SEQ ID NO:71, SEQ ID NO:74, SEQ ID NO:75, SEQ ID NO:77, SEQ ID NO:80, SEQ ID NO:81, SEQ ID NO:84, SEQ ID NO:85, SEQ ID NO:86, SEQ ID NO:87, SEQ ID NO:90, SEQ ID NO:91, SEQ ID NO:92, SEQ ID NO:93, SEQ ID NO:106, SEQ ID NO:108, SEQ ID NO:112, SEQ ID NO:115, SEQ ID NO:116, SEQ ID NO:126, SEQ ID NO:127, SEQ ID NO:128, SEQ ID NO:129, SEQ ID NO:131, SEQ ID NO:132, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:152, SEQ ID NO:159, SEQ ID NO:162, SEQ ID NO:163, SEQ ID NO:164, and SEQ ID NO:165. The peptide is preferably not hecate-1, anubis-1, anubis-2, anubis-5, anubis-8, vishnu-1, vishnu-2, vishnu-3, vishnu-8, or shiva-10.

The peptide can be similar to any of the above described peptides, and preferably is similar to SEQ ID NO:2 (or SEQ ID NO:16 or SEQ ID NO:126), SEQ ID NO:4 (or SEQ ID NO:14 or SEQ ID NO:17), SEQ ID NO:25, SEQ ID NO:43, SEQ ID NO:75, SEQ ID NO:84, SEQ ID NO:115, SEQ ID NO:126, or SEQ ID NO:132 as determined by percent identity. The percent identity between the peptides is preferably at least about 70%, 75%, 80%, 85%, 90%, 95%, 96%, 97%, 98%, 99%, or 100%. Percent identity is determined using a sequence alignment by the commercial product CLUSTALW. The number of aligned amino acids are divided by the length of the shorter peptide, and the result is multiplied by 100% to determine percent identity. If the length of the shorter peptide is less than 10 amino acids, the number of aligned amino acids are divided by 10, and the result is multiplied by 100% to determine percent identity.

The peptides can comprise D- or L-amino acids. The peptides can comprise all D-amino acids. The peptides can have an acid C-terminus (—CO₂H) or an amide C-terminus (—CONH₂, —CONHR, or —CONR₂).

Methods of Use

An additional embodiment of the invention is directed towards methods of using the above described peptides. The methods of use preferably do not cause injury or kill normal uninfected mammalian cells. The methods of use at therapeutic dose levels preferably do not cause injury to or kill normal uninfected or non-neoplastic mammalian cells. The methods of use may involve the use of a single peptide, or may involve the use of multiple peptides.

An embodiment of the invention is the use of the above described peptides to inhibit or kill microbial cells (microorganisms). The microorganisms may be bacterial cells, fungal cells, protozoa, viruses, or eucaryotic cells infected with pathogenic microorganisms. The method generally is directed towards the contacting of microorganisms with the peptide. The contacting step can be performed in vivo, in vitro, topically, orally, transdermally, systemically, or by any other method known to those of skill in the art. The contacting step is preferably performed at a concentration sufficient to inhibit or kill the microorganisms. The concentration of the peptide can be at least about 0.1 μM, at least about 0.5 μM, at least about 1 μM, at least about 10 [M, at least about 20 μM, at least about 50 μM, or at least about 100 μM. The methods of use can be directed towards the inhibition or killing of microorganisms such as bacteria, gram positive bacteria, gram negative bacteria, mycobacteria, yeast, fungus, algae, protozoa, viruses, and intracellular organisms. Specific examples include, but are not limited to, Staphylococcus, Staphylococcus aureus, Pseudomonas, Pseudomonas aeruginosa, Escherichia coli, Chlamydia, Candida albicans, Saccharomyces, Saccharomyces cerevisiae, Schizosaccharomyces pombe, Trypanosoma cruzi, or Plasmodium falciparum. The contacting step can be performed by systemic injection, oral, subcutaneous, IP, IM, IV injection, or by topical application. For injection, the dosage can be between any of the following concentrations: about 1 mg/kg, about 5 mg/kg, about 10 mg/kg, about 25 mg/kg, about 50 mg/kg, about 75 mg/kg, and about 100 mg/kg. The contacting step can be performed on a mammal, a cat, a dog, a cow, a horse, a pig, a bird, a chicken, a plant, a fish, or a human.

Presently preferred peptides for antibacterial applications include SEQ ID NO:1, SEQ ID NO:2, SEQ ID NO:4, SEQ ID NO:6, SEQ ID NO:8, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:23, SEQ ID NO:25, SEQ ID NO:26, SEQ ID NO:27, SEQ ID NO:28, SEQ ID NO:30, SEQ ID NO:31, SEQ ID NO:32, SEQ ID NO:34, SEQ ID NO:35, SEQ ID NO:36, SEQ ID NO:41, SEQ ID NO:43, SEQ ID NO:45, SEQ ID NO:46, SEQ ID NO:50, SEQ ID NO:51, SEQ ID NO:52, SEQ ID NO:55, SEQ ID NO:56, SEQ ID NO:57, SEQ ID NO:58, SEQ ID NO:60, SEQ ID NO:65, SEQ ID NO:66, SEQ ID NO:67, SEQ ID NO:68, SEQ ID NO:74, SEQ ID NO:75, SEQ ID NO:77, SEQ ID NO:80, SEQ ID NO:81, SEQ ID NO:84, SEQ ID NO:86, SEQ ID NO:87, SEQ ID NO:93, SEQ ID NO:106, SEQ ID NO:108, SEQ ID NO:112, SEQ ID NO:115, SEQ ID NO:126, SEQ ID NO:128, SEQ ID NO:162, SEQ ID NO:163, SEQ ID NO:164, and SEQ ID NO:165.

Presently preferred peptides for antifungal applications include SEQ ID NO:2, SEQ ID NO:10, SEQ ID NO:11, SEQ ID NO:12, SEQ ID NO:13, SEQ ID NO:16, SEQ ID NO:17, SEQ ID NO:25, SEQ ID NO:30, SEQ ID NO:35, SEQ ID NO:58, SEQ ID NO:66, SEQ ID NO:67, SEQ ID NO:80, SEQ ID NO:81, SEQ ID NO:84, SEQ ID NO:85, SEQ ID NO:86, SEQ ID NO:106, SEQ ID NO:108, SEQ ID NO:115, SEQ ID NO:116, SEQ ID NO:126, SEQ ID NO:128, SEQ ID NO:131, SEQ ID NO:143, SEQ ID NO:163, and SEQ ID NO:165.

An additional embodiment of the invention is the use of any of the above described peptides to inhibit or kill cancer cells. The method generally is directed towards the contacting of cancer cells with the peptide. The contacting step can be performed in vivo, in vitro, topically, orally, transdermally, systemically, or by any other method known to those of skill in the art. The contacting step is preferably performed at a concentration sufficient to inhibit or kill the cancer cells. The concentration of the peptide can be at least about at least about 0.1 μM, at least about 0.5 μM, at least about 1 μM, at least about 10 μM, at least about 20 μM, at least about 50 μM, or at least about 100 μM. The cancer cells can generally be any type of cancer cells. The cancer cells can be sarcomas, lymphomas, carcinomas, leukemias, breast cancer cells, colon cancer cells, skin cancer cells, ovarian cancer cells, cervical cancer cells, testicular cancer cells, lung cancer cells, prostate cancer cells, and skin cancer cells. The contacting step can be performed by subcutaneous, IP injection, IM injection, IV injection, direct tumor injection, or topical application. For injection, the dosage can be between any of the following concentrations: about 0.1 mg/kg, about 1 mg/kg, about 5 mg/kg, about 10 mg/kg, about 25 mg/kg, about 50 mg/kg, about 75 mg/kg, and about 100 mg/kg. The contacting step can be performed on a mammal, a cat, a dog, a cow, a horse, a pig, a bird, a chicken, a plant, a fish, a goat, a sheep, or a human. The inhibition of cancer cells can generally be any inhibition of growth of the cancer cells as compared to the cancer cells without peptide treatment. The inhibition is preferably at least about 10%, 20%, 30%, 40%, 50%, 60%, 70%, 80%, 90%, 95%, 96%, 97%, 98%, 99%, and ideally 100% inhibition of growth. The inhibition may be achieved by lysis of the cancer cells or by other means. The cancer inhibiting peptide can be used synergistically with other cancer chemotherapeutic agents.

Presently preferred peptides for anticancer applications include SEQ ID NO:1, SEQ ID NO:2, SEQ ID NO:8, SEQ ID NO:11, SEQ ID NO:13, SEQ ID NO:16, SEQ ID NO:17, SEQ ID NO:18, SEQ ID NO:19, SEQ ID NO:20, SEQ ID NO:25, SEQ ID NO:26, SEQ ID NO:29, SEQ ID NO:30, SEQ ID NO:32, SEQ ID NO:35, SEQ ID NO:46, SEQ ID NO:51, SEQ ID NO:56, SEQ ID NO:57, SEQ ID NO:58, SEQ ID NO:60, SEQ ID NO:68, SEQ ID NO:75, SEQ ID NO:86, SEQ ID NO:152, and SEQ ID NO:162

An additional embodiment of the invention is directed towards a method for promoting the stimulation and/or proliferation of cells. The method can comprise contacting the cells and a composition, wherein the composition comprises a peptide. The peptide can be any of the above described peptides. The concentration of the peptide in the composition can be about 0.01 μM to about 500 μM, about 0.1 μM to about 100 μM, about 1 μM to about 50 μM, or about 1 μM to about 10 μM. The cells can generally be any type of cells, and preferably are mammalian cells, specifically including, but not limited to fibroblast and leukocyte cells, including lymphocyte and phagocytic cells. The metabolic stimulation and/or proliferation of the cells is preferably increased by at least about 10%, 20%, 30%, 40%, 50%, 60%, 70%, 80%, 90%, 100%, 125%, 150%, 175%, or 200% relative to the same cells not contacted with the composition. The composition can further comprise a growth factor. The stimulatory and proliferative properties of some of the FLAK peptides hold promise for their application in skin care, wound healing, and in immunomodulation of compromised mammalian immune systems.

Presently preferred peptides for stimulation and proliferation applications include SEQ ID NO:1, SEQ ID NO:2, SEQ ID NO:5, SEQ ID NO:6, SEQ ID NO:8, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:20, SEQ ID NO:27, SEQ ID NO:28, SEQ ID NO:30, SEQ ID NO:32, SEQ ID NO:34, SEQ ID NO:45, SEQ ID NO:46, SEQ ID NO:50, SEQ ID NO:51, SEQ ID NO:55, SEQ ID NO:56, SEQ ID NO:57, SEQ ID NO:58, SEQ ID NO:59, SEQ ID NO:60, SEQ ID NO:61, SEQ ID NO:65, SEQ ID NO:66, SEQ ID NO:71, SEQ ID NO:74, SEQ ID NO:75, SEQ ID NO:77, SEQ ID NO:80, SEQ ID NO:81, SEQ ID NO:87, SEQ ID NO:90, SEQ ID NO:91, SEQ ID NO:92, SEQ ID NO:108, SEQ ID NO:115, SEQ ID NO:116, SEQ ID NO:126, SEQ ID NO:127, SEQ ID NO:129, SEQ ID NO:132, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:159, SEQ ID NO:162, SEQ ID NO:164, and SEQ ID NO:165.

An additional embodiment of the invention is directed towards a method for promoting wound healing of skin or ocular and internal body tissues damaged by normal aging, disease, injury, or by surgery or other medical procedures. The method can comprise administering to the wound of an animal a composition, wherein the composition comprises any of the above described peptides. The concentration of the peptide in the composition can be about 0.01 μM to about 500 μM, about 0.1 μM to about 100 μM, about 1 μM to about 50 μM, or about 1 μM to about 10 μM. The composition can be administered to the wound topically or by systemic delivery. The animal can generally be any kind of animal, preferably is a mammal, and more preferably is a human, cow, horse, cat, dog, pig, goat, or sheep. The promotion of wound healing is preferably at least 10%, 20%, 30%, 40%, 50%, 60%, 70%, 80%, 90%, 100%, 125%, 150%, 175%, or 200% relative to the same wound not contacted with the composition.

Presently preferred peptides for wound healing applications include SEQ ID NO:1, SEQ ID NO:2, SEQ ID NO:5, SEQ ID NO:6, SEQ ID NO:8, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:20, SEQ ID NO:27, SEQ ID NO:28, SEQ ID NO:30, SEQ ID NO:32, SEQ ID NO:34, SEQ ID NO:45, SEQ ID NO:46, SEQ ID NO:50, SEQ ID NO:51, SEQ ID NO:55, SEQ ID NO:56, SEQ ID NO:57, SEQ ID NO:58, SEQ ID NO:59, SEQ ID NO:60, SEQ ID NO:61, SEQ ID NO:65, SEQ ID NO:66, SEQ ID NO:71, SEQ ID NO:74, SEQ ID NO:75, SEQ ID NO:77, SEQ ID NO:80, SEQ ID NO:81, SEQ ID NO:87, SEQ ID NO:90, SEQ ID NO:91, SEQ ID NO:92, SEQ ID NO:93, SEQ ID NO:115, SEQ ID NO:116, SEQ ID NO:126, SEQ ID NO:127, SEQ ID NO:129, SEQ ID NO:132, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:159, SEQ ID NO:162, and SEQ ID NO:164.

A further embodiment of the invention is directed towards methods for the additive or synergistic enhancement of the activity of a therapeutic agent. The method can comprise preparing a composition, wherein the composition comprises a peptide and a therapeutic agent. Alternatively, the method may comprise co-therapy treatment with a peptide (or peptides) used in conjunction with other therapeutic agents. The peptide can be any of the above described peptides. The therapeutic agent can generally be any therapeutic agent, and preferably is an antibiotic, an antimicrobial agent, a growth factor, a chemotherapy agent, an antimicrobial agent, lysozyme, a chelating agent, or EDTA. Preferably, the activity of the composition is higher than the activity of the same composition containing the therapeutic agent but lacking the peptide. The composition or co-therapy can be used in in vitro, in vivo, topical, oral, IV, IM, IP, and transdermal applications. The enhancement of the activity of the composition containing the therapeutic agent and the peptide is preferably at least 10%, 20%, 30%, 40%, 50%, 60%, 70%, 80%, 90%, 100%, 125%, 150%, 175%, or 200% relative to the activity of the therapeutic agent alone.

Generally, any peptide which is active on a stand-alone basis against a target is preferred for use to increase either additively or synergistically the activity of another therapeutic agent against that target. If several peptides are candidates for a given synergy application, then the less toxic peptides would be more favorably considered.

A further additional embodiment of the invention is directed towards methods for the treatment of patients diagnosed with Cystic Fibrosis (CF). CF causes, among other effects, inflammation and infection in the lungs. The above described peptides of the instant invention can be used in treating such lung infections, which are often caused by P. aeruginosa. The inventive peptides may possess anti-inflammatory properties, making them further useful for the treatment of lung infections in CF patients. The peptide can be administered to the CF patient by any acceptable method including inhalation or systemic delivery. The peptide can be administered in a single dose, in multiple doses, or as a continuous delivery.

An additional embodiment of the invention is directed towards methods of treating sexually transmitted diseases (STDs). Many of the fungal species responsible for STDs are inhibited or killed by the inventive peptides described above. Examples of such species include C. albicans, C. glabrata, and C. tropicalis. The inventive peptides may additionally be used against other agents responsible for STDs including viruses and bacteria. The peptides can be administered to an STD patient by any acceptable method, such as topical, oral, or systemic delivery. The peptide can be administered in a single dose, in multiple doses, or as a continuous delivery. The peptide can be administered in any acceptable form, such as a cream, gel, or liquid.

A further additional embodiment of the invention is directed towards methods for the treatment of acne. The inventive peptides have activity against the bacteria isolated from acne sores, Propionibacterium acnes, and may further possess anti-inflamatory properties. The peptide can be present in a clinical therapeutic composition or in a cosmeceutical composition. The peptide can be administered in any acceptable form, such as a cream, gel, or liquid. The peptide can be administered in any acceptable manner, such as topical administration. The peptide can be used in a treatment method, or in a preventative manner to reduce or eliminate future outbreaks of acne.

Yet a further embodiment is directed towards cosmetic compositions. The inventive peptides have been shown to stimulate collagen and fibroblasts, and to promote wound healing. The inclusion of the inventive peptides in cosmetic formulations may be useful in the anti-aging and rejuvination markets.

An additional embodiment of the invention is directed towards the use of peptides in promoting wound healing. The inventive peptides have high potency against the bacteria most associated with wound infections: S. aureus, S. pyogenes, and P. aeruginosa. The peptides also promote wound healing and reducing of inflammation. The peptide can be administered in any acceptable form, such as a cream, gel, or liquid. The peptide can be administered in any acceptable manner, such as topical administration or systemic administration.

The following Examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

### Microbial Strains

The following table lists the various microorganisms used throughout the Examples.

TABLE 2

| Microorganism | Reference or source |
|---|---|
| Escherichia coli | ATCC25922 |
| Staphylococcus aureus | ATCC6538 and ATCC25923 |
| Pseudomonas aeruginosa | ATCC9027 and ATCC27853 |
| Staphylococcus intermedius | ATCC19930 and ATCC20034 |
| Candida albicans | ATCC10231 |
| Escherichia coli UB1005 | D. Clark, FEMS Microb. Lett. 21: 189-195, 1984 |
| Salmonella typhimurium 14028S | Fields et al., Science 243: 1059-1062, 1989 |
| Staphylococcus aureus SAP0017 | Methicillin resistant clinical isolate from Prof. T. |
| | Chow, Vancouver General hospital |
| Staphylococcus epidermidis C621 | clinical isolate from David. Speer |
| Streptococcus pyogenes | ATCC19615 |
| Streptococcus pyogenes M76 | From Prof. R. Gallo (UCSD) |
| Streptococcus pneumoniae | ATCC6305-C718 |
| Streptococcus pneumoniae | ATCC49619-C719 |
| Pseudomonas aeruginosa H187 | Angus, et al., AAC 21: 299-309, 1982 |
| Pseudomonas aeruginosa H374 | Masuda, N., et al., AAC, 36: 1847-1851, 1992 |
| (nfxB efflux mutant) | |
| Pseudomonas aeruginosa H744 | Poole, K., et al. J. Bacteriol. 175-7363-7372, 1993 |
| nalB multiple resistant efflux | |
| mutant | |
| Pseudomonas aeruginosa 100609 | Tobramycin resistant strain from Prof. D. Woods |
| | (U. Calgary) |
| Pseudomonas aeruginosa 105663 | Tobramycin resistant strain from Prof. D. Woods |
| | (U. Calgary) |
| Candida albicans 105 | From Prof Barbara Dill (UBC) |
| Candida guilliermondii | ATCC8492 |
| Candida tropicalis | ATCC13803 |
| Candida glabrata | ATCC15126 |
| Propionibacterium acnes | ATCC6919 |
| Propionibacterium acnes | ATCC11827 |
| Acinetobacter baumannii | ATCC19606 |

### Example 2

### Antimicrobial Assays I

The data for the following antimicrobial assay of the peptides have been obtained by making OD measurements in in vitro cell culture experiments with and without added peptide. The protocol used is as follows.

Cell lines included Staphylococcus aureus ATCC 6538 or 25923, Pseudomonas aeruginosa ATCC 9027 or 27853. Medium used were Antibiotic Medium 3 (Difco), Antibiotic Medium 2 (Difco), and 0.85% saline. Controls used were physiological saline, and gentamycin at 50, 25, 10, 5, 1, and 0.1 ppm.

The preparation of all media, stock solutions, and dilutions took place in a laminar flow hood to prevent contamination. Bacterial cells were freshly grown on antibiotic medium 2 agar slants (pH 7.0 at 25° C.). Bacteria were suspended and diluted in antibiotic medium 3 to about 10⁴ cfu/ml and used as the inoculum. Sample solutions (100 μl/well) were added to plates according to the plate layout. Inoculum (100 μl/well) was added to achieve a final concentration of 5×10³ cfu/ml. Negative controls received 100 μl saline and 100 μl growth medium. Positive controls received 100 μl saline and 100 μl inoculum. Bacterial plates were incubated at 37° C. for 24 hours.

Absorbance was read at 620 nm after shaking to resuspend cells. The minimum inhibitory concentration (MIC) was defined as the lowest concentration of peptide that completely inhibits the growth of the test organism.

The yeast assay was performed in RPMI 1640 media (pH 7.0 at 25° C.).

The data presented in Table 3 were obtained using the above protocol. However, the data for Table 4 were obtained with a modified protocol wherein the medium was tryptic soy broth, inocolum strength was approximately 10⁴ CFU per ml, and values determined were minimum bactericidal concentrations (MBC) or minimum fungicidal concentrations (MFC).

The following Table 3 describes the antimicrobial properties of the peptides measured as MIC or MFC values in μg/mL. Staph6538 is Staphylococcus aureus ATCC accession number 6538; paerug9027 is Pseudomonas aeruginosa ATCC accession number 9027, yeast is Saccharomyces cerevisiae.

TABLE 3

| Name | SEQ ID NO: | P Number | staph6538 | paerug9027 | yeast |
|---|---|---|---|---|---|
| Hecate AC #1010 | 1 | 1 | 5 | 10 | > |
| Hecate AM | 2 | 2 | 25 | 100 | 25 |
| SB-37 AC #1018 | 3 | 5 | 100 | 50 | > |
| SB-37 AM | 5 | 12 | > | 100 | > |
| Shiva 10 AC #1015 | 6 | 13 | 10 | > | > |
| FLAK01 AM | 8 | 23 | 5 | 50 | 100 |
| FLAK04 AM | 10 | 25 | 10 | 5 | 25 |
| FLAK05 AM | 11 | 26 | 10 | 15 | > |
| FLAK06 AM | 12 | 27 | 10 | 10 | 25 |
| KAL V | 15 | 30 | > | > | ND |
| FLAK 17 AM | 16 | 34 | 5 | 50 | 25 |
| FLAK 26 AM | 17 | 35 | 5 | 200 | 25 |
| Hecate 2DAc | 19 | 37 | 5 | 100 | 50 |
| FLAK43 AM | 20 | 38 | 5 | 50 | 50 |
| FLAK44 AM | 21 | 39 | 100 | 25 | 100 |
| FLAK62 AM | 22 | 40 | 100 | 25 | 100 |
| FLAK 06R-AM | 23 | 41 | 10 | 10 | ND |
| MSI-78 AM | 24 | 42 | 10 | > | 200 |
| FLAK50 | 25 | 43 | 5 | 100 | 25 |
| FLAK51 | 26 | 44 | 5 | 5 | 50 |
| FLAK57 | 27 | 45 | 5 | 100 | 100 |
| FLAK71 | 28 | 46 | 10 | 5 | 50 |
| FLAK77 | 29 | 47 | 200 | 100 | 50 |
| FLAK50V | 30 | 48 | 5 | 5 | 25 |
| FLAK50F | 31 | 49 | 10 | 200 | 50 |
| FLAK26V AM | 32 | 50 | 5 | 15 | 50 |
| CAME-15 | 33 | 53 | 5 | 15 | 50 |
| FLAK50C | 34 | 54 | 5 | 50 | 50 |
| FLAK50D | 35 | 55 | 5 | 5 | 25 |
| FLAK 50E | 36 | 56 | 200 | 5 | 50 |
| FLAK80 | 37 | 57 | 100 | 200 | 200 |
| FLAK81 | 38 | 58 | 100 | 100 | 200 |
| FLAK82 | 39 | 59 | > | > | > |
| FLAK83M | 40 | 60 | 200 | 100 | 200 |
| FLAK 17 C | 43 | 64 | 5 | > | 200 |
| FLAK 50H | 44 | 65 | 15 | 50 | 200 |
| FLAK 50G | 45 | 66 | 5 | 50 | 100 |
| Shiva deriv P69 + KWKL | 46 | 70 | 10 | > | 100 |
| Shiva 10 (1-18_AC | 47 | 71 | 15 | 15 | 200 |
| CA(1-7)Shiva10(1-16) | 49 | 73 | 50 | 15 | 100 |
| FLAK 54 | 50 | 74 | 15 | 5 | 100 |
| FLAK 56 | 51 | 75 | 5 | 5 | 50 |
| FLAK 58 | 52 | 76 | 10 | 100 | 200 |
| FLAK 72 | 53 | 77 | 200 | 100 | 200 |
| FLAK 75 | 54 | 79 | 100 | 200 | 100 |
| Shiva 10 (1-16) Ac | 55 | 80 | 10 | 100 | 100 |
| CA(1-7)Shiva10(1-16)-COOH | 56 | 81 | 10 | > | > |
| Indolocidin-ac | 57 | 91 | 10 | > | > |
| FLAK50B | 58 | 92 | 5 | 5 | 50 |
| FLAK50I | 60 | 94 | 10 | > | > |
| FLAK50K | 61 | 95 | 100 | 200 | > |
| FLAK50L | 62 | 96 | > | > | > |
| Shiva-11 | 63 | 98 | > | > | > |
| Shiva 11[(1-16)ME(2-9)]-COOH | 64 | 99 | 100 | > | > |
| FLAK 50N | 65 | 101 | 10 | 25 | 100 |
| FLAK 50O | 66 | 102 | 5 | 10 | 50 |
| FLAK 50P | 67 | 103 | 10 | 25 | 100 |
| CA(1-&Hecate(11/23) | 68 | 104 | 10 | 10 | 200 |
| PYL-ME | 69 | 105 | 200 | 200 | > |
| FLAG26-D1 | 70 | 106 | 100 | 25 | 100 |
| Vishnu3 | 71 | 107 | > | > | > |
| Melittin | 72 | 108 | 5 | > | 25 |
| FLAK26-D2 | 73 | 109 | > | 200 | 200 |
| FLAG26-D3 | 74 | 110 | > | 200 | 200 |
| FLAK50 Q1 | 75 | 111 | 5 | 100 | 200 |
| FLAK50 Q2 | 76 | 112 | 50 | 200 | 100 |
| FLAK50 Q3 | 77 | 113 | 10 | 200 | 200 |
| FLAK50 Q4 | 78 | 114 | 50 | 15 | 100 |
| FLAK50 Q5 | 79 | 117 | 100 | 200 | 200 |
| FLAK50 Q6 | 80 | 118 | 10 | 100 | 100 |
| FLAK50 Q7 | 81 | 119 | 50 | 25 | 50 |
| FLAK50 Q8 | 82 | 120 | 50 | 200 | 200 |
| FLAK50 Q9 | 83 | 121 | 50 | > | 100 |
| FLAK50 T1 | 85 | 123 | 50 | 200 | 100 |
| FLAK50 T2 | 86 | 124 | 5 | 100 | 100 |
| FLAK50 T3 | 87 | 125 | 10 | 100 | 50 |
| FLAK50 T4 | 88 | 126 | > | > | > |
| FLAK50 T5 | 89 | 127 | 100 | 25 | 100 |
| FLAK90 | 90 | 128 | > | 100 | 200 |
| FLAK91 | 91 | 129 | 100 | 25 | 100 |
| FLAK92 | 92 | 130 | 200 | 200 | 200 |
| FLAK93 | 93 | 131 | 25 | 10 | 100 |
| FLAK50 Z1 | 94 | 132 | > | 100 | > |
| FLAK50 Z2 | 95 | 133 | > | > | > |
| FLAK50 Z3 | 96 | 134 | 100 | > | 200 |
| FLAK50 Z4 | 97 | 135 | 15 | 10 | 50 |
| FLAK50 Z5 | 98 | 136 | 100 | 50 | 100 |
| FLAK50 Z6 | 99 | 137 | > | > | > |
| FLAK50 Z7 | 100 | 138 | > | > | > |
| FLAK50 Z8 | 101 | 139 | 50 | 25 | 200 |
| FLAK50 Z9 | 102 | 140 | > | > | > |
| FLAK94 | 103 | 141 | 15 | 50 | 200 |
| FLAK93B | 104 | 142 | 100 | 50 | 100 |
| FLAK50 Z10 | 105 | 143 | 100 | 50 | 200 |
| FLAK96 | 106 | 144 | 5 | 50 | 50 |
| FLAK97 | 107 | 145 | 200 | 100 | 200 |
| FLAK98 | 108 | 146 | 10 | 10 | 50 |
| FKRLA | 109 | 147 | 5 | 5 | 200 |
| FLAK91B | 110 | 148 | > | 200 | 200 |
| FLAK92B | 111 | 149 | 50 | 100 | 200 |
| FLAK99 | 112 | 150 | 100 | 10 | > |
| FLAK50T6 | 113 | 151 | > | > | 200 |
| FLAK50T7 | 114 | 152 | 100 | 50 | 100 |
| FLAK95 | 115 | 153 | 5 | 25 | 100 |
| FLAK50T8 | 116 | 154 | 100 | 100 | 50 |
| FLAK50T9 | 117 | 155 | > | > | > |
| FLAK100-CO2H | 118 | 156 | 15 | > | > |
| FAGVL | 119 | 157 | 200 | > | > |
| FLAK120 | 126 | 165 | 10 | 25 | 25 |
| FLAK121 | 127 | 166 | > | > | > |
| FLAK96B | 128 | 167 | 10 | 25 | 100 |
| FLAK96G | 129 | 168 | 50 | 100 | > |
| FLAK96F | 130 | 169 | 100 | 100 | 100 |
| FLAK96C | 131 | 170 | 200 | 100 | 100 |
| FLAK96D | 132 | 171 | 25 | 50 | 100 |
| FLAK 96 | 137 | 176 | > | > | > |
| FLAK 96J | 139 | 178 | 200 | 100 | > |
| FLAK 96L | 140 | 179 | 50 | 50 | 100 |
| FLAK-120G | 141 | 180 | 200 | > | > |
| FLAK-120D | 142 | 181 | 100 | 200 | 100 |
| FLAK-120C | 143 | 182 | > | > | > |
| FLAK-120B | 144 | 183 | 200 | 100 | 200 |
| FLAK-120F | 145 | 184 | 25 | 100 | 100 |
| FLAK 50M | 165 | 97 | 5 | 50 | 50 |

> indicates greater than 200 μg/mL;
ND = not determined.

The following Table 4 describes describes the antimicrobial properties of the peptides measured as minimum bactericidal or minimum fungicidal (Candida) concentrations. MBC or MFC values are in μg/mL. E. coli is Escherichia coli ATCC accession number 25922; P. aerug is Pseudomonas aeruginosa ATCC accession number 27853, S. aur. is Stapholococcus aureus ATCC accession number 25923; Candida is Candida albicans ATCC accession number 10231.

TABLE 4

| | | E. coli | P. aerug | S. aur | Candida |
|---|---|---|---|---|---|
| SEQ ID NO: | P # | A.25922 | A.27853 | A.25923 | A.10231 |
| 1 | 1 | 25 | 30 | 25 | >50 |
| 2 | 2 | 25 | 10 | 25 | >50 |
| 3 | 5 | 50 | >60 | 40 | ND |
| 4 | 11 | 40 | 25 | 25 | >50 |
| 5 | 12 | 50 | >60 | 75 | ND |
| 6 | 13 | 8 | 15 | 30 | >50 |
| 8 | 23 | 15 | 25 | 30 | >50 |
| 9 | 24 | >80 | 30 | >40 | >50 |
| 10 | 25 | 40 | 30 | 40 | >50 |
| 11 | 26 | >80 | >40 | >40 | >50 |
| 12 | 27 | 10 | 8 | 8 | >50 |
| 13 | 27B | 40 | 10 | >40 | >40 |
| 14 | 27C | 10 | 4 | >40 | >40 |
| 15 | 30 | 10 | 15 | 40 | >50 |
| 16 | 34 | 15 | 15 | 40 | >40 |
| 17 | 35 | 8 | 8 | 10 | >40 |
| 18 | 36 | 30 | 15 | 10 | >40 |
| 19 | 37 | 8 | 8 | 40 | >50 |
| 20 | 38 | 15 | 30 | 15 | ND |
| 21 | 39 | >40 | >40 | >40 | ND |
| 22 | 40 | 30 | 40 | >40 | ND |
| 23 | 41 | 40 | 40 | 40 | ND |
| 24 | 42 | 10 | 30 | 10 | ND |
| 25 | 43 | 8 | 15 | 4 | 15 |
| 26 | 44 | 10 | 55 | 30 | >50 |
| 27 | 45 | 30 | 40 | 80 | >50 |
| 29 | 47 | >50 | >50 | >50 | >50 |
| 30 | 48 | 8 | 25 | 4 | 10 |
| 31 | 49 | 40 | 30 | 50 | 30 |
| 32 | 50 | 50 | 25 | 25 | >50 |
| 33 | 53 | 15 | 15 | 10 | 30 |
| 34 | 54 | 15 | 40 | 15 | 30 |
| 35 | 55 | 4 | 10 | 4 | 25 |
| 36 | 56 | 50 | 10 | 55 | 30 |
| 37 | 57 | >50 | >50 | >50 | >50 |
| 38 | 58 | >50 | >50 | >50 | >50 |
| 39 | 59 | >50 | >50 | >50 | >50 |
| 40 | 60 | >50 | >50 | >50 | >50 |
| 41 | 61 | 4 | 50 | >80 | >40 |
| 42 | 63 | 10 | 50 | 15 | 60 |
| 43 | 64 | 10 | 30 | 4 | >50 |
| 44 | 65 | >55 | >50 | >55 | >50 |
| 45 | 66 | 40 | 50 | 30 | 40 |
| 46 | 70 | 40 | 30 | 40 | >50 |
| 47 | 71 | 50 | 40 | >50 | >50 |
| 48 | 72 | >50 | 40 | >50 | >50 |
| 50 | 74 | >55 | 50 | >55 | >55 |
| 51 | 75 | 40 | 30 | >55 | 30 |
| 52 | 76 | 40 | >55 | >55 | >50 |
| 53 | 77 | >50 | >50 | >50 | >50 |
| 54 | 79 | >50 | >50 | >50 | >50 |
| 55 | 80 | 30 | 15 | >50 | >50 |
| 58 | 92 | 40 | 25 | 15 | 25 |
| 59 | 93 | >50 | >50 | >50 | >50 |
| 60 | 94 | >50 | >50 | >50 | >50 |
| 61 | 95 | >50 | >50 | >50 | >50 |
| 62 | 96 | >50 | >50 | >50 | >50 |
| 65 | 101 | 300 | >50 | >50 | 40 |
| 66 | 102 | 25 | 30 | 25 | 15 |
| 67 | 103 | 30 | 30 | >50 | 25 |
| 69 | 105 | 25 | >50 | ND | >50 |
| 70 | 106 | 50 | >50 | ND | >50 |
| 71 | 107 | ND | >50 | >50 | >50 |
| 72 | 108 | >50 | >50 | 25 | >50 |
| 73 | 109 | ND | ND | 80 | >50 |
| 74 | 110 | 8 | >50 | >50 | >50 |
| 75 | 111 | 30 | ND | 40 | INACT |
| 76 | 112 | 30 | INACT | INACT | INACT |
| 77 | 113 | INACT | INACT | INACT | 40 |
| 79 | 117 | INACT | INACT | INACT | INACT |
| 80 | 118 | 8 | 25 | | |
| 81 | 119 | 15 | 30 | 4 | 25 |
| 82 | 120 | INACT | INACT | INACT | INACT |
| 83 | 121 | INACT | INACT | INACT | 50 |
| 84 | 122 | 30 | 30 | 25 | 15 |
| 85 | 123 | 40 | INACT | INACT | 25 |
| 86 | 124 | 10 | 40 | 8 | 15 |
| 87 | 125 | 40 | 40 | INACT | 40 |
| 88 | 126 | INACT | INACT | INACT | INACT |
| 89 | 127 | INACT | INACT | INACT | INACT |
| 90 | 128 | INACT | INACT | INACT | INACT |
| 91 | 129 | INACT | INACT | INACT | INACT |
| 92 | 130 | INACT | INACT | INACT | INACT |
| 93 | 131 | INACT | INACT | INACT | INACT |
| 94 | 132 | INACT | INACT | INACT | INACT |
| 95 | 133 | INACT | INACT | INACT | INACT |
| 96 | 134 | INACT | INACT | INACT | INACT |
| 97 | 135 | INACT | 40 | INACT | 25 |
| 98 | 136 | INACT | INACT | INACT | INACT |
| 99 | 137 | INACT | INACT | INACT | INACT |
| 100 | 138 | INACT | INACT | INACT | INACT |
| 101 | 139 | INACT | INACT | INACT | INACT |
| 102 | 140 | INACT | INACT | INACT | INACT |
| 103 | 141 | INACT | INACT | INACT | INACT |
| 104 | 142 | INACT | INACT | INACT | INACT |
| 105 | 143 | INACT | INACT | INACT | INACT |
| 106 | 144 | 10 | 25 | 25 | 25 |
| 107 | 145 | INACT | INACT | INACT | 100 |
| 108 | 146 | 10 | >250 | 75 | 10 |
| 109 | 147 | 25 | 75 | >250 | >250 |
| 110 | 148 | 150 | >250 | >250 | 100 |
| 111 | 149 | 150 | >250 | >250 | 100 |
| 112 | 150 | 75 | >250 | >250 | 50 |
| 113 | 151 | >250 | >250 | >250 | 100 |
| 114 | 152 | 150 | 150 | >250 | 50 |
| 115 | 153 | 10 | 25 | 5 | 25 |
| 116 | 154 | 50 | 100 | >250 | 25 |
| 117 | 155 | >250 | >250 | >250 | >250 |
| 118 | 156 | 100 | >250 | >250 | >250 |
| 119 | 157 | 75 | >250 | >250 | >250 |
| 120 | 159 | 10 | 10 | >250 | 50 |
| 121 | 160 | >250 | >250 | >250 | >250 |
| 122 | 161 | 150 | >250 | >250 | 25 |
| 123 | 162 | 50 | >250 | >250 | 100 |
| 124 | 163 | 25 | 50 | 25 | 25 |
| 125 | 164 | 25 | 25 | 25 | 25 |
| 126 | 165 | 10 | 25 | 25 | 10 |
| 127 | 166 | >250 | >250 | >250 | >250 |
| 128 | 167 | 25 | >250 | 10 | 25 |
| 129 | 168 | 75 | 100 | >250 | 150 |
| 130 | 169 | 200 | >250 | >250 | 75 |
| 131 | 170 | 25 | >250 | 150 | 25 |
| 132 | 171 | 75 | 100 | >250 | 50 |
| 133 | 172 | >250 | >250 | >250 | >250 |
| 134 | 173 | >250 | >250 | >250 | 150 |
| 162 | 67 | 25 | 30 | 30 | >50 |
| 165 | 97 | 25 | >50 | 25 | 25 |

INACT refers to no detectable activity.
ND indicates no data available.

### Example 3

### Antimicrobial Assays II

Anti-microbial activity against a broader range of pathogens (including clinical strains) than were tested in Example 2. It should be noted that somewhat different protocols were employed for the assays in Example 2 and Example 3.

MICs were determined for this Example using a slightly modified version of the NCCLS (National Committee for Clinical Laboratory Standards) broth microdilution method as described previously (Steinberg et al., AAC 41: 1738, 1997). Briefly, antimicrobial agents were prepared as 10× concentrates in the most appropriate solvent. For the peptide, 0.01% acetic acid containing 0.2% bovine serum albumin as a carrier protein was used. Inocula were prepared by resuspending colonies from a BAP in medium and adjusting the suspension to match that of a 0.5 McFarland standard. The suspension was diluted into fresh medium (as recommended by NCCLS for the organism) to give 2×10⁵ to 7×10⁵ CFU/ml for bacteria or 2×10³ to 7×10³ CFU/ml for Candida. After dispensing 100 μl aliquots of the microbial suspension into each well of a 96-well polypropylene microtiter plate, 11 μl of test compound was added. The MIC was defined as the lowest concentration of drug which prevented visible turbidity after 16 to 20 hours (bacteria) or 46 to 50 hours (Candida) at 35° C. For facultative anaerobes incubation was performed in 7% carbon dioxide and for strict anaerobes in an oxygen free environment maintained using a standard anaerobic “jar”. All MICs were performed three times and the mean value determined.

TABLE 5
Activity against gram positive bacteria

| Peptide | S. aureus | | |
|---|---|---|---|
| (SEQ ID NO:) | (MRSA) | S. epidermidis C621 | S. pyogenes M76 |
| P23 (8) | 32 | 16 | 16 |
| P25 (10) | 16 | 4 | 8 |
| P26 (11) | 32 | 4 | 4 |
| P27 (12) | 16 | 4 | 4 |
| P34 (16) | 16 | 8 | 4 |
| P35 (17) | 8 | 4 | 4 |
| P37 (19) | 8 | 4 | 8 |
| P41 (23) | 64 | 4 | 8 |
| P42 (24) | 16 | 2 | 4 |
| P43 (25) | 4 | 2 | 2 |
| P44 (26) | 8 | 4 | 4 |
| P46 (28) | 64 | 8 | 8 |
| P49 (31) | 64 | 8 | 8 |
| P50 (32) | 4 | 4 | 8 |
| P54 (34) | 16 | 8 | 8 |
| P55 (35) | 4 | 2 | 4 |
| P59 (39) | 8 | 8 | 2 |
| P60 (40) | 32 | 4 | 8 |
| P61 (41) | 32 | 8 | 16 |
| P63* (42) | 32 | 16 | 8 |
| P64* (43) | 8 | 4 | 4 |
| P72 (48) | 16 | 4 | 16 |
| P73 (49) | 16 | 4 | 16 |
| P75 (51) | 32 | 8 | 8 |
| P94* (60) | 16 | 8 | 8 |
| P97 (165) | 8 | 4 | 4 |
| P105* (69) | 32 | 8 | 16 |
| P111 (75) | 8 | 4 | 4 |
| P119 (81) | 8 | 4 | 8 |
| P124 (86) | 8 | 4 | 16 |
| P146 (108) | 16 | 8 | 8 |
| P153 (115) | 16 | 4 | 2 |
| P157 (119) | 32 | 4 | 8 |
| P177 (138) | 8 | 4 | 8 |
| P301 (147) | 8 | 4 | 8 |
| P504 (155) | 4 | 4 | 8 |
| P510 (161) | 8 | 4 | 8 |
| P2 (2) | 32 | 8 | 4 |
| P27 (12) | 8 | 4 | 4 |

Bold indicates broad spectrum activity;
*indicates gram-positive selective

TABLE 6
Activity against gram positive bacteria

| Peptide | | | | |
|---|---|---|---|---|
| (SEQ ID NO:) | S. pyogenes | S. pneumoniae | S. pneumoniae | P. acne |
| P23 (8) | 8 | 16 | 16 | 4 |
| P25 (10) | 8 | 64 | 8 | 2 |
| P26 (11) | 4 | >128 | 16 | 4 |
| P27 (12) | 4 | 32 | 8 | 4 |
| P34 (16) | 4 | 8 | 8 | 8 |
| P35 (17) | 16 | 4 | | 4 |
| P37 (19) | 8 | 64 | 16 | 4 |
| P41 (23) | 8 | 64 | 32 | 4 |
| P42 (24) | 4 | 32 | 8 | 2 |
| P43 (25) | 2 | 8 | 4 | 2 |
| P44 (26) | 4 | 8 | 16 | 4 |
| P46 (28) | 16 | 64 | 128 | |
| P49 (31) | 8 | 64 | 32 | |
| P50 (32) | 4 | 32 | 16 | 4 |
| P54 (34) | 8 | 64 | 64 | |
| P55 (35) | 2 | 8 | 4 | 4 |
| P59 (39) | 2 | 16 | 4 | 2 |
| P60 (40) | 8 | 128 | >128 | 4 |
| P61 (41) | 16 | 128 | 32 | 2 |
| P63* (42) | 8 | 128 | 16 | |
| P64* (43) | 4 | 8 | 2 | 2 |
| P72 (48) | 16 | >128 | 16 | 2 |
| P73 (49) | 16 | >128 | 64 | 4 |
| P75 (51) | 4 | >128 | 64 | 16 |
| P94* (60) | 8 | 64 | 128 | |
| P97 (165) | 4 | 32 | 16 | 8 |
| P105* (69) | 16 | 64 | 32 | 16 |
| P111 (75) | 2 | 16 | 4 | 4 |
| P119 (81) | 8 | 128 | 32 | 8 |
| P124 (86) | 16 | >128 | 64 | 8 |
| P146 (108) | 8 | >128 | 128 | 16 |
| P153 (115) | 2 | 32 | 8 | 4 |
| P157 (119) | 8 | 128 | 16 | 4 |
| P177 (138) | 4 | 32 | 16 | 8 |
| P301 (147) | 8 | >128 | 8 | 2 |
| P504 (155) | 16 | 64 | 8 | 4 |
| P510 (161) | 8 | 64 | 16 | 2 |
| P2A* (2) | 8 | 128 | 32 | |
| P97 (165) | 8 | 32 | 32 | 16 |
| P27 (12) | 4 | 16 | 4 | 4 |

Bold indicates broad spectrum activity;
*indicates gram-positive selective;
S. pyogenes ATCC19615;
S. pneumoniae C718;
S. pneumoniae C719;
P. acne ATCC 6919

TABLE 7
Activity against gram-negative bacteria

| | E. coli | S. typhimurium | P. aeruginosa |
|---|---|---|---|
| Peptide (SEQ ID NO:) | UB1005 | 14028S | H374 |
| P12 (5) | 1 | 4 | 8 |
| P39 (21) | 4 | 16 | 16 |
| P41 (23) | 2 | 4 | 4 |
| P46 (28) | 4 | 8 | 4 |
| P61 (41) | 2 | 4 | 4 |
| P71 (47) | 2 | 8 | 4 |
| P100 (163) | 0.5 | 4 | 8 |
| P109 (73) | 16 | 32 | 8 |
| P110 (74) | 16 | 32 | 8 |
| P157 (119) | 8 | 8 | 8 |
| P306 (150) | 4 | 4 | 8 |
| P46 (28) | 8 | 16 | 4 |
| P29 (14) | 8 | 8 | 16 |

TABLE 8
Activity against gram-negative bacteria

| | P. aeruginosa | C. glabrata |
|---|---|---|
| Peptide | H187 | ATCC15126 |
| P12 (5) | 16 | 128 |
| P39 (21) | 32 | 16 |
| P41 (23) | 8 | 32 |
| P46 (28) | 16 | 32 |
| P61 (41) | 8 | 32 |
| P71 (47) | 8 | 32 |
| P100 (163) | 32 | >128 |
| P109 (73) | 64 | 128 |
| P110 (74) | 64 | 128 |
| P157 (119) | 8 | 64 |
| P306 (150) | 16 | >128 |
| P46 (28) | 8 | 32 |
| P29 (14) | 32 | 128 |

TABLE 9
Activity against Pseudomonas bacterial strains

| Peptide | P. | P. | | P. |
|---|---|---|---|---|
| (SEQ | aeruginosa | aeruginosa | P. aeruginosa | aeruginosa |
| ID NO:) | H374 | H187 | Tb 105663 | Tb 100609 |
| P12 (5) | 8 | 16 | 8 | 8 |
| P25 (10) | 8 | 8 | 8 | 8 |
| P27 (12) | 8 | 8 | 16 | 16 |
| P35 (17) | 8 | 8 | 4 | 4 |
| P37 (19) | 8 | 8 | 16 | 16 |
| P39 (21) | 16 | 32 | 32 | 32 |
| P41 (23) | 4 | 8 | 8 | 8 |
| P42 (24) | 4 | 8 | 8 | 8 |
| P43 (25) | 8 | 8 | 8 | 8 |
| P44 (26) | 8 | 8 | 16 | 8 |
| P45 (27) | 8 | 16 | 32 | 32 |
| P46 (28) | 4 | 16 | 32 | 16 |
| P50 (32) | 4 | 4 | 8 | 4 |
| P55 (35) | 8 | 8 | 16 | 8 |
| P59 (39) | 8 | 8 | 8 | 8 |
| P61 (41) | 4 | 8 | 8 | 16 |
| P71 (47) | 4 | 8 | 16 | 16 |
| P72 (48) | 4 | 8 | 8 | 8 |
| P73 (49) | 8 | 16 | 16 | 16 |
| P97 (165) | 8 | 16 | 16 | 16 |
| P111 (75) | 8 | 8 | 32 | 16 |
| P119 (81) | 8 | 16 | 16 | 16 |
| P124 (86) | 16 | 32 | 64 | 64 |
| P146 (108) | 2 | 4 | 8 | 8 |
| P153 (115) | 4 | 8 | 8 | 8 |
| P157 (119) | 8 | 8 | 16 | 16 |
| P177 (138) | 16 | 16 | 32 | 32 |
| P301 (247) | 4 | 8 | 8 | 8 |
| P306 (150) | 8 | 16 | 32 | 16 |
| P504 (155) | 8 | 8 | 16 | 8 |
| P510 (161) | 8 | 8 | 16 | 16 |
| P2 (2) | 16 | 16 | 16 | 32 |
| P13 (6) | 16 | 16 | 16 | 16 |
| P27 (12) | 8 | 8 | 8 | 8 |
| P11 (4) | 16 | 16 | 16 | 16 |

Bold indicates broad spectrum activity.

The following tables compare the anti-fungal and anti-bacterial properties of a representative sample of peptides.

TABLE 10
Comparison of anti-fungal and anti-bacterial activities of selected peptides

| Peptide | | C. tropicalis | C. glabrata |
|---|---|---|---|
| (SEQ ID NO:) | C. albicans 105 | ATCC13803 | ATCC15126 |
| P40 (22) | 32 | 1 | 32 |
| P47 (29) | 32 | 1 | 64 |
| P49 (31) | 16 | 2 | 16 |
| P74 (50) | 16 | 1 | 16 |
| P77 (53) | 16 | 1 | 64 |
| P79 (54) | 32 | 2 | 128 |
| P101 (65) | 32 | 4 | 32 |
| P103 (67) | 16 | 2 | 16 |
| P106 (70) | 32 | 2 | 64 |
| P113 (77) | 32 | 4 | 32 |
| P122 (84) | 32 | 4 | 64 |
| P154 (116) | 64 | 8 | 128 |
| P167 (128) | 64 | 8 | 128 |
| P169 (130) | 64 | 8 | 128 |

TABLE 11
Comparison of anti-fungal and anti-bacterial activities of selected
peptides

| Peptide | E. coli | S. typhimurium | P. aeruginosa | S. aureus |
|---|---|---|---|---|
| (SEQ ID NO:) | UB1005 | 14028S | H187 | SAP0017 |
| P40 (22) | 64 | >128 | >128 | >128 |
| P47 (29) | 64 | >128 | 64-128 | >128 |
| P49 (31) | 32 | 64 | 16-64 | 64 |
| P74 (50) | 16 | 64 | 32-128 | >128 |
| P77 (53) | 64 | >128 | 64-128 | >128 |
| P79 (54) | 32 | >128 | >128 | >128 |
| P101 (65) | 32 | 128 | 32-128 | 128 |
| P103 (67) | 32 | 128 | 64 | 64 |
| P106 (70) | 64 | >128 | >128 | >128 |
| P113 (77) | 32 | 44 | 32-128 | 32 |
| P122 (84) | 64 | 128 | 32-128 | 128 |
| P154 (116) | 64 | >128 | >128 | >128 |
| P167 (128) | 32 | 64 | 128 | 128 |
| P169 (130) | 32 | 64 | 128 | >128 |

Many of the disclosed FLAK peptides have activity against a wide array of microorganisms. The following tables illustrate these properties for a representative sample of peptides.

TABLE 12
Broad spectrum activities

| | | S. | | |
|---|---|---|---|---|
| Peptide | E. coli | typhimurium | P. aeruginosa | P. aeruginosa |
| (SEQ ID NO:) | UB1005 | 1402S | H374 | H187 |
| P25 (10) | 8 | 8 | 8 | 8 |
| P27 (12) | 8 | 16 | 8 | 8 |
| P35 (17) | 2 | 4 | 8 | 8 |
| P37 (19) | 4 | 8 | 8 | 8 |
| P42 (24) | 4 | 8 | 4 | 8 |
| P43 (25) | 8 | 8 | 8 | 8 |
| P44 (26) | 1 | 4 | 8 | 8 |
| P45 (27) | 4 | 32 | 8 | 16 |
| P50 (32) | 2 | 4 | 4 | 4 |
| P55 (35) | 4 | 4 | 8 | 8 |
| P59 (39) | 8 | 8 | 8 | 8 |
| P72 (48) | 2 | 8 | 4 | 8 |
| P73 (49) | 8 | 16 | 8 | 16 |
| P97 (165) | 8 | 16 | 8 | 16 |
| P111 (75) | 16 | 16 | 8 | 8 |
| P119 (81) | 4 | 8 | 8 | 16 |
| P124 (86) | 16 | 16 | 16 | 32 |
| P146 (108) | 2 | 4 | 2 | 4 |
| P153 (115) | 8 | 8 | 4 | 8 |
| P177 (138) | 8 | 16 | 16 | 16 |
| P301 (147) | 8 | 8 | 4 | 8 |
| P504 (155) | 4 | 4 | 8 | 8 |
| P510 (161) | 8 | 16 | 8 | 8 |

TABLE 13
Broad spectrum activities

| Peptide | S. aureus | S. epidermis | C. albicans | C. glabrata |
|---|---|---|---|---|
| (SEQ ID NO:) | SAP0017 | C621 | 105 | ATCC15126 |
| P25 (10) | 16 | 4 | 32 | 32 |
| P27 (12) | 16 | 4 | 32 | 32 |
| P35 (17) | 8 | 4 | 32 | 16 |
| P37 (19) | 8 | 4 | 32 | 32 |
| P42 (24) | 16 | 2 | 32 | 64 |
| P43 (25) | 4 | 2 | 8 | 16 |
| P44 (26) | 8 | 4 | 8 | 16 |
| P45 (27) | 32 | 16 | 16 | 16 |
| P50 (32) | 4 | 4 | 16 | 16 |
| P55 (35) | 4 | 2 | 16 | 8 |
| P59 (39) | 8 | 8 | 32 | 16 |
| P72 (48) | 16 | 4 | 32 | 64 |
| P73 (49) | 16 | 4 | 32 | 128 |
| P97 (165) | 8 | 4 | 16 | 16 |
| P111 (75) | 8 | 4 | 32 | 32 |
| P119 (81) | 8 | 4 | 16 | 16 |
| P124 (86) | 8 | 4 | 16 | 16 |
| P146 (108) | 16 | 8 | 8 | 16 |
| P153 (115) | 16 | 4 | 16 | 16 |
| P177 (138) | 8 | 4 | 16 | 16 |
| P301 (147) | 8 | 4 | 32 | 32 |
| P504 (155) | 4 | 4 | 64 | 64 |
| P510 (161) | 8 | 4 | 32 | 64 |
| P27 (12) | 8 | 4 | 16 | 16 |

While FLAK peptides are generally active against an array of microbial targets, not all peptides are equally effective against all microorganisms. The following tables present some combinations of peptides and microorganisms in which the peptide was observed to have poor activity.

TABLE 14
Low observed anti-microbial activities

| Peptide | E. coli | S. typhimurium | P. aeruginosa |
|---|---|---|---|
| (SEQ ID NO:) | UB1005 | 14028S | H374 |
| P57 (37) | >128 | >128 | >128 |
| P58 (38) | >128 | >128 | >128 |
| P65 (44) | 128 | >128 | 64 |
| P76 (52) | 16 | 128 | 64 |
| P93 (59) | 128 | >128 | 128 |
| P95 (61) | >128 | >128 | >128 |
| P96 (62) | >128 | >128 | >128 |
| P107 (71) | >128 | >128 | >128 |
| P112 (76) | >128 | >128 | >128 |
| P114 (78) | 32 | 128 | >128 |
| P120 (82) | >128 | >128 | 128 |
| P121 (83) | >128 | >128 | >128 |
| P123 (85) | 64 | >128 | >128 |
| P126 (88) | >128 | >128 | >128 |
| P127 (89) | 128 | >128 | >128 |
| P128 (90) | 128 | >128 | >128 |
| P129 (91) | 64 | >128 | >128 |
| P130 (92) | >128 | >128 | >128 |
| P131 (93) | >128 | >128 | >128 |
| P132 (94) | 128 | >128 | >128 |
| P133 (95) | >128 | >128 | >128 |
| P134 (96) | 128 | >128 | 128 |
| P136 (98) | 128 | >128 | >128 |
| P137 (99) | >128 | >128 | >128 |
| P138 (100) | >128 | >128 | >128 |
| P139 (101) | 64 | >128 | >128 |
| P140 (102) | >128 | >128 | >128 |
| P141 (103) | >128 | >128 | >128 |
| P142 (104) | 64 | 128 | >128 |
| P143 (105) | >128 | >128 | >128 |
| P145 (107) | >128 | >128 | >128 |
| P147 (109) | 64 | 128 | 128 |
| P148 (110) | 128 | >128 | >128 |
| P149 (111) | 32 | >128 | 128 |
| P151 (113) | >128 | >128 | 128 |
| P152 (114) | 32 | >128 | >128 |
| P155 (117) | >128 | >128 | >128 |
| P166 (127) | >128 | >128 | >128 |
| P168 (129) | 128 | >128 | 128 |
| P169 (130) | 64 | 64 | 128 |
| P170 (131) | 64 | >128 | >128 |
| P171 (132) | 32 | >128 | >128 |
| P174 (135) | >128 | >128 | >128 |
| P175 (136) | >128 | >128 | >128 |
| P180 (141) | >128 | >128 | >128 |

TABLE 15
Low observed anti-microbial activities

| | P. | | S. | |
|---|---|---|---|---|
| Peptide | aeruginosa | S. aureus | epidermidis | C. albicans |
| (SEQ ID NO:) | H187 | SAP0017 | C621 | 105 |
| P57 (37) | >128 | >128 | >128 | 128 |
| P58 (38) | >128 | >128 | >128 | 64 |
| P65 (44) | >128 | >128 | >128 | 64 |
| P76 (52) | >128 | >128 | >128 | 64 |
| P93 (59) | >128 | >128 | >128 | 64 |
| P95 (61) | >128 | >128 | >128 | >128 |
| P96 (62) | >128 | >128 | >128 | >128 |
| P107 (71) | >128 | >128 | >128 | >128 |
| P112 (76) | >128 | >128 | 64 | 128 |
| P114 (78) | >128 | >128 | 64 | 64 |
| P120 (82) | >128 | >128 | >128 | 64 |
| P121 (83) | >128 | >128 | >128 | 64 |
| P123 (85) | >128 | >128 | 16 | 64 |
| P126 (88) | >128 | >128 | >128 | >128 |
| P127 (89) | >128 | >128 | 64 | 32 |
| P128 (90) | >128 | >128 | 128 | 128 |
| P129 (91) | >128 | >128 | 32 | 128 |
| P130 (92) | >128 | >128 | >128 | >128 |
| P131 (93) | >128 | >128 | >128 | >128 |
| P132 (94) | >128 | >128 | >128 | 128 |
| P133 (95) | >128 | >128 | >128 | >128 |
| P134 (96) | >128 | >128 | 128 | 64 |
| P136 (98) | >128 | >128 | 128 | 64 |
| P137 (99) | >128 | >128 | >128 | >128 |
| P138 (100) | >128 | >128 | >128 | >128 |
| P139 (101) | 128 | >128 | 64 | 128 |
| P140 (102) | >128 | >128 | >128 | >128 |
| P141 (103) | >128 | >128 | >128 | >128 |
| P142 (104) | >128 | >128 | 128 | 64 |
| P143 (105) | >128 | >128 | >128 | >128 |
| P145 (107) | >128 | >128 | >128 | 64 |
| P147 (109) | >128 | >128 | 64 | 64 |
| P148 (110) | >128 | >128 | 128 | 128 |
| P149 (111) | >128 | >128 | >128 | 128 |
| P151 (113) | >128 | >128 | >128 | 128 |
| P152 (114) | >128 | >128 | 32 | 128 |
| P155 (117) | >128 | >128 | >128 | >128 |
| P166 (127) | >128 | >128 | >128 | >128 |
| P168 (129) | 128 | >128 | 128 | 128 |
| P169 (130) | >128 | >128 | 32 | 64 |
| P170 (131) | >128 | 0.128 | >128 | 128 |
| P171 (132) | >128 | >128 | 128 | >128 |
| P174 (135) | >128 | >128 | >128 | >128 |
| P175 (136) | >128 | >128 | >128 | >128 |
| P180 (141) | >128 | >128 | >128 | >128 |

### Example 4

### Anti-Cancer Assays

Cancer cell assays were performed in a manner similar to the anti-microbial assays described above, except that the assay procedure used the MTT dye protocol. Viability of cells is determined by the dye response. In the following procedure, approximately 1.5×10⁴ cells per well were added and viability was determined with the cells in a semi-confluent state. The assay was performed in a 96-well microtiter plate. After addition of peptide, the plate was set for 24 hours. MTT (5 mg/ml in phenol red-free RPMI-1640, 20 μl) was added to each well including positive control wells untreated with peptide. The plate was incubated at 37° C. for 4 hours. The liquid contents of each well was removed, and isopropanol with 0.1 M HCl (100 μl) was added to each well. The plate was sealed with parafilm to prevent evaporation of the isopropanol. The plate is allowed to rest for 5-10 minutes in order to solubilize the precipitate. Purified water (100 μl) was added to each well. Absorbance was determined with an ELISA Reader instrument. Color intensity at 540 nm is proportional to viability of cells. Results for each concentration of peptide are plotted relative to untreated controls, and LD50 values are determined from the graphs.

WI38 (ATCC No. CCL75) is a normal fibroblast line of lung diploid cells, MCF7 (ATCC No. HTB22) is a breast adenocarcinoma tumor cell line, SW480 (ATCC No. CCL228) is a colon adenocarcinoma tumor cell line, BMKC is a cloned melanoma line derived from Bowes melanoma line HMCB (ATCC No. CRL9607), H1299 (ATCC No. CRL5803) is a lung large cell carcinoma tumor line, HeLaS3 (ATCC No. CCL2.2) is a cervical epitheleal carcinoma tumor cell line, and PC3 (ATCC No. CRL1435) is a prostate adenocarcinoma tumor cell line. Numbers are LD₅₀ values (μg/mL). Data on the six targets are presented in the following Tables 16 and 17.

TABLE 16

| | SEQ | | | | | |
|---|---|---|---|---|---|---|
| Name | ID NO: | P No. | WI38 | MCF7 | SW480 | BMKC |
| HECATE AC | 1 | 1 | 27 | 54 | 6 | 72 |
| HECATE AM | 2 | 2 | 66 | 23 | 46 | 128 |
| SB37COOH | 3 | 5 | 130 | 175 | 82 | 120 |
| SB-37 AM | 5 | 12 | 950 | 540 | > | > |
| SHIVA 10 AC | 6 | 13 | 57 | > | ND | ND |
| FLAK01 AM | 8 | 23 | 34 | 62 | 5 | 27 |
| FLAK03 AM | 9 | 24 | 55 | 26 | 38 | 85 |
| FLAK04 AM | 10 | 25 | 24 | 10 | 12 | 36 |
| FLAK05 AM | 11 | 26 | 96 | 74 | 8 | 94 |
| FLAK06 AM | 12 | 27 | 37 | 14 | 26 | 44 |
| FLAK06 AC | 13 | 27B | 101 | 65 | 59 | 93 |
| FLAK06 R-AC | 14 | 27C | 520 | 140 | 210 | 300 |
| KAL V | 15 | 30 | 93 | 72 | 62 | 140 |
| FLAK 17 AM | 16 | 34 | 40 | 21 | 35 | 53 |
| FLAK 26 AM | 17 | 35 | 8 | 9 | 14 | 7 |
| FLAK 25 AM | 18 | 36 | 19 | 9 | 30 | 56 |
| HECATE 2DAc | 19 | 37 | 80 | 14 | 57 | 150 |
| FLAK43 AM | 20 | 38 | 12 | 17 | 13 | 21 |
| FLAK44 AM | 21 | 39 | 300 | 130 | 435 | 510 |
| FLAK62 AM | 22 | 40 | > | 760 | > | > |
| FLAK 06R-AM | 23 | 41 | 175 | 98 | 120 | 290 |
| MSI-78 AM | 24 | 42 | 67 | 31 | 34 | 140 |
| FLAK50 | 25 | 43 | 5 | 9 | 9 | 7 |
| FLAK51 | 26 | 44 | 36 | 140 | 32 | 47 |
| FLAK57 | 27 | 45 | 200 | 260 | 180 | 160 |
| FLAK71 | 28 | 46 | 200 | 300 | 160 | 150 |
| FLAK77 | 29 | 47 | > | 575 | > | 700 |
| FLAK50V | 30 | 48 | 41 | 23 | 47 | 43 |
| FLAK50F | 31 | 49 | 135 | 40 | 100 | 115 |
| FLAK26V AM | 32 | 50 | 43 | 32 | 46 | 40 |
| CAME-15 | 33 | 53 | 32 | 45 | | 40 |
| FLAK50C | 34 | 54 | 97 | 60 | | 90 |
| FLAK50D | 35 | 55 | 32 | 16 | 14 | 16 |
| FLAK 50E | 36 | 56 | 250 | 500 | 215 | 205 |
| FLAK80 | 37 | 57 | 900 | > | 740 | 740 |
| FLAK81 | 38 | 58 | > | > | > | > |
| FLAK82 | 39 | 59 | 77 | 31 | 42 | 155 |
| FLAK83M | 40 | 60 | > | > | > | > |
| FLAK 26 Ac | 41 | 61 | 93 | 105 | 100 | 140 |
| INDOLICIDIN | 42 | 63 | ND | 64 | 345 | 200 |
| FLAK 17 C | 43 | 64 | 37 | 80 | | 35 |
| FLAK 50H | 44 | 65 | 320 | 475 | 345 | 250 |
| FLAK 50G | 45 | 66 | 240 | 90 | 145 | 200 |
| SHIVA DERIV P69 + KWKL | 46 | 70 | 34 | 44 | 11 | 94 |
| SHIVA 10 (1-18_AC | 47 | 71 | 355 | 190 | 250 | 445 |
| SHIVA 10 PEPTIDE 71 + KWKL | 48 | 72 | 125 | 93 | 82 | 290 |
| CA(1-7)Shiva10(1-16) | 49 | 73 | 160 | 150 | 70 | 360 |
| FLAK 54 | 50 | 74 | 335 | 465 | 340 | 460 |
| FLAK 56 | 51 | 75 | 80 | 42 | 17 | 24 |
| FLAK 58 | 52 | 76 | 445 | 970 | 400 | 750 |
| FLAK 72 | 53 | 77 | > | > | > | 125 |
| FLAK 75 | 54 | 79 | > | 540 | > | 830 |
| SHIVA 10 (1-16) Ac | 55 | 80 | 28 | 29 | 35 | 76 |
| CA(1-7)Shiva10(1-16)-COOH | 56 | 81 | 8 | 63 | 13 | 12 |
| INDOLOCIDIN-ac | 57 | 91 | 9 | 12 | 30 | 180 |
| FLAK50B | 58 | 92 | 43 | 23 | 51 | 46 |
| FLAK50I | 60 | 94 | 6 | 65 | ND | 11 |
| FLAK50K | 61 | 95 | 250 | > | > | 820 |
| FLAK50L | 62 | 96 | > | > | > | > |
| Shiva-11 | 63 | 98 | 47 | 96 | 125 | 94 |
| SHIVA 11 [(1-16)ME(2-9]-COOH | 64 | 99 | 34 | 95 | 120 | 94 |
| FLAK 50N | 65 | 101 | 300 | 250 | 170 | 160 |
| FLAK 50O | 66 | 102 | 73 | 60 | 57 | 60 |
| FLAK 50P | 67 | 103 | 26 | 46 | 90 | 75 |
| CA(1-&HECATE(11/23) | 68 | 104 | 24 | 11 | 54 | 100 |
| PYL-ME | 69 | 105 | 430 | 635 | > | ND |
| FLAG26-D1 | 70 | 106 | > | 620 | 570 | 690 |
| VISHNU3 | 71 | 107 | > | > | > | > |
| MELITTIIN | 72 | 108 | 16 | 9 | 23 | 18 |
| FLAK26-D2 | 73 | 109 | > | > | > | > |
| FLAG26-D3 | 74 | 110 | 45 | 180 | 325 | 400 |
| FLAK50 Q1 | 75 | 111 | 24 | 35 | 27 | 26 |
| FLAK50 Q2 | 76 | 112 | 420 | 500 | 800 | 445 |
| FLAK50 Q3 | 77 | 113 | 170 | 150 | 180 | 115 |
| FLAK50 Q4 | 78 | 114 | > | 730 | > | > |
| FLAK50 Q5 | 79 | 117 | > | > | > | > |
| FLAK50 Q6 | 80 | 118 | 170 | 70 | 115 | 135 |
| FLAK50 Q7 | 81 | 119 | 45 | 54 | 46 | 36 |
| FLAK50 Q8 | 82 | 120 | 600 | 730 | 630 | 660 |
| FLAK50 Q9 | 83 | 121 | 625 | 400 | 800 | 670 |
| FLAK50 Q10 | 84 | 122 | 720 | 360 | 570 | 700 |
| FLAK50 T1 | 85 | 123 | 600 | 615 | > | 635 |
| FLAK50 T2 | 86 | 124 | 21 | 18 | 9 | 10 |
| FLAK50 T3 | 87 | 125 | 90 | 90 | 125 | 220 |
| FLAK50 T4 | 88 | 126 | > | > | > | > |
| FLAK50 T5 | 89 | 127 | 760 | 440 | 400 | 535 |
| FLAK90 | 90 | 128 | 500 | 500 | 530 | 330 |
| FLAK91 | 91 | 129 | > | > | 550 | > |
| FLAK92 | 92 | 130 | > | > | > | > |
| FLAK93 | 93 | 131 | > | 600 | 555 | > |
| FLAK50 Z1 | 94 | 132 | > | > | > | > |
| FLAK50 Z2 | 95 | 133 | > | > | > | > |
| FLAK50 Z3 | 96 | 134 | > | > | 740 | > |
| FLAK50 Z4 | 97 | 135 | 110 | 54 | 80 | 155 |
| FLAK50 Z5 | 98 | 136 | > | 500 | 600 | 530 |
| FLAK50 Z6 | 99 | 137 | > | > | > | > |
| FLAK50 Z7 | 100 | 138 | > | > | > | > |
| FLAK50 Z8 | 101 | 139 | 550 | 625 | > | 525 |
| FLAK50 Z9 | 102 | 140 | > | > | > | > |
| FLAK94 | 103 | 141 | 420 | 430 | 560 | 465 |
| FLAK93B | 104 | 142 | 73 | 44 | 38 | 38 |
| FLAK50 Z10 | 105 | 143 | > | > | > | > |
| FLAK96 | 106 | 144 | 750 | 150 | 285 | 250 |
| FLAK97 | 107 | 145 | > | > | > | > |
| FLAK98 | 108 | 146 | 270 | 110 | 380 | 185 |
| FKRLA | 109 | 147 | 83 | 106 | 185 | 110 |
| FLAK91B | 110 | 148 | 380 | 315 | > | 330 |
| FLAK92B | 111 | 149 | > | > | > | > |
| FLAK99 | 112 | 150 | 125 | 160 | 235 | 190 |
| FLAK50T6 | 113 | 151 | > | > | > | > |
| FLAK50T7 | 114 | 152 | 620 | 430 | 740 | > |
| FLAK95 | 115 | 153 | 130 | 64 | 61 | 165 |
| FLAK50T8 | 116 | 154 | 600 | 315 | 750 | 330 |
| FLAK50T9 | 117 | 155 | > | > | > | > |
| FLAK100-CO2H | 118 | 156 | 230 | 135 | 345 | 520 |
| FAGVL | 119 | 157 | 500 | 240 | 530 | 600 |
| Modelin-5 | 120 | 159 | 82 | 61 | 140 | 140 |
| Modelin-5-CO2H | 121 | 160 | 700 | 320 | 370 | 220 |
| FLAK120 | 126 | 165 | 470 | 360 | 240 | 240 |
| FLAK121 | 127 | 166 | > | > | > | > |
| FLAK96B | 128 | 167 | 260 | 230 | 360 | 240 |
| FLAK96G | 129 | 168 | > | 630 | > | 590 |
| FLAK96F | 130 | 169 | > | 510 | > | 530 |
| FLAK96C | 131 | 170 | > | 940 | > | > |
| FLAK96D | 132 | 171 | 615 | 305 | 770 | 600 |
| Modelin-8D | 135 | 174 | > | > | > | > |
| Modelin-8E | 136 | 175 | > | > | 70 | > |
| Flak 96H | 137 | 176 | > | > | > | > |
| Flak 96I | 138 | 177 | 270 | 190 | 310 | 310 |
| Flak 96J | 139 | 178 | 405 | 770 | > | 640 |
| Flak 96L | 140 | 179 | 540 | 555 | > | 920 |
| FLAK-120G | 141 | 180 | 940 | 950 | 600 | 770 |
| FLAK-120D | 142 | 181 | 500 | 550 | 870 | 830 |
| FLAK-120C | 143 | 182 | > | > | > | > |
| FLAK-120B | 144 | 183 | > | > | > | > |
| FLAK-120F | 145 | 184 | 800 | 260 | 440 | 600 |
| Magainin2wisc | 146 | 300 | 52 | 22 | 60 | 130 |
| D2A21 | 147 | 301 | 66 | 64 | 76 | 140 |
| KSL-1 | 148 | 302 | 800 | 340 | > | 700 |
| KSL-7 | 149 | 303 | 355 | 315 | 530 | 330 |
| LSB-37 | 150 | 306 | 320 | 50 | 240 | 170 |
| Anubis-2 | 151 | 307 | 75 | 38 | 73 | 83 |
| FLAK 17 CV | 152 | 501 | 26 | 23 | ND | ND |
| FLAK50 Q1V | 153 | 502 | 64 | 92 | ND | ND |
| D2A21V | 154 | 503 | 150 | 210 | ND | ND |
| FLAK 25 AM V | 155 | 504 | 110 | 130 | ND | ND |
| FLAK43 AM V | 156 | 505 | 85 | 86 | ND | ND |
| FLAK50D V | 157 | 506 | 75 | 45 | ND | ND |
| HECATE AM V | 158 | 507 | 285 | 340 | ND | ND |
| HECATE AC V | 159 | 508 | 190 | 160 | ND | ND |
| FLAK04 AM V | 160 | 509 | 95 | 84 | ND | ND |
| 03 AMV | 161 | 510 | 77 | 62 | ND | ND |
| D-Shiva 10 AC | 162 | 67 | 4 | 7 | ND | ND |
| Shiva 11 AC | 163 | 100 | 95 | 175 | 82 | 120 |
| Shiva 10(1-18)AM | 164 | 69 | 101 | 45 | 63 | 66 |

Note:
> indicates greater than 1000;
ND indicates not determined;
numbers are in μg/mL.

TABLE 17

| | SEQ ID | | | | | |
|---|---|---|---|---|---|---|
| Name | NO: | P No. | WI38 | H1299 | HeLaS3 | PC3 |
| HECATE AC | 1 | 1 | 27 | 44 | 95 | 61 |
| HECATE AM | 2 | 2 | 66 | 140 | 50 | 44 |
| SB37COOH | 3 | 5 | 130 | 220 | 150 | ND |
| SB-37 AM | 5 | 12 | 950 | 720 | > | 630 |
| SHIVA 10 AC | 6 | 13 | 57 | > | > | 83 |
| FLAK01 AM | 8 | 23 | 34 | 64 | 82 | 41 |
| FLAK03 AM | 9 | 24 | 55 | 72 | 145 | 38 |
| FLAK04 AM | 10 | 25 | 24 | 37 | 20 | 12 |
| FLAK05 AM | 11 | 26 | 96 | 84 | 150 | 125 |
| FLAK06 AM | 12 | 27 | 37 | 16 | 25 | 8 |
| FLAK06 AC | 13 | 27B | 101 | 54 | 80 | 16 |
| FLAK06 AM | 14 | 27C | 520 | 170 | 260 | 280 |
| KAL V | 15 | 30 | 93 | 125 | 190 | 65 |
| FLAK 17 AM | 16 | 34 | 40 | 24 | 62 | 9 |
| FLAK 26 AM | 17 | 35 | 8 | 16 | 27 | 5 |
| FLAK 25 AM | 18 | 36 | 19 | 57 | ND | 19 |
| HECATE 2DAc | 19 | 37 | 80 | 150 | ND | 64 |
| FLAK43 AM | 20 | 38 | 12 | 33 | 35 | 10 |
| FLAK44 AM | 21 | 39 | 300 | 420 | 620 | 310 |
| FLAK62 AM | 22 | 40 | > | > | > | 435 |
| FLAK 06R-AM | 23 | 41 | 175 | 245 | 185 | 140 |
| MSI-78 AM | 24 | 42 | 67 | 150 | ND | 66 |
| FLAK50 | 25 | 43 | 5 | 6 | 15 | 12 |
| FLAK51 | 26 | 44 | 36 | 72 | 22 | 45 |
| FLAK57 | 27 | 45 | 200 | 330 | 160 | 170 |
| FLAK71 | 28 | 46 | 200 | 290 | 280 | 280 |
| FLAK77 | 29 | 47 | > | > | > | > |
| FLAK50V | 30 | 48 | 41 | 17 | 44 | 32 |
| FLAK50F | 31 | 49 | 135 | 140 | ND | 77 |
| FLAK26V AM | 32 | 50 | 43 | 7 | 33 | 54 |
| CAME-15 | 33 | 53 | 32 | 65 | 30 | 40 |
| FLAK50C | 34 | 54 | 97 | 80 | 190 | 90 |
| FLAK50D | 35 | 55 | 32 | 7 | 15 | 47 |
| FLAK 50E | 36 | 56 | 250 | 370 | 300 | 435 |
| FLAK80 | 37 | 57 | 900 | > | 830 | > |
| FLAK81 | 38 | 58 | > | > | > | > |
| FLAK82 | 39 | 59 | 77 | 180 | ND | 81 |
| FLAK83M | 40 | 60 | > | > | > | > |
| FLAK 26 Ac | 41 | 61 | 93 | 127 | 170 | 66 |
| INDOLICIDIN | 42 | 63 | ND | 270 | 345 | 290 |
| FLAK 17 C | 43 | 64 | 37 | 30 | 30 | 46 |
| FLAK 50H | 44 | 65 | 320 | 450 | 210 | 470 |
| FLAK 50G | 45 | 66 | 240 | 130 | 140 | 170 |
| SHIVA DERIV P69 + KWKL | 46 | 70 | 34 | 63 | 28 | 82 |
| SHIVA 10 (1-18_AC | 47 | 71 | 355 | 320 | 570 | 270 |
| SHIVA 10 PEPTIDE 71 + KWKL | 48 | 72 | 125 | 160 | 240 | 63 |
| CA(1-7)Shiva10(1-16) | 49 | 73 | 160 | 115 | 270 | 97 |
| FLAK 54 | 50 | 74 | 335 | 670 | 260 | 660 |
| FLAK 56 | 51 | 75 | 80 | 80 | 74 | 54 |
| FLAK 58 | 52 | 76 | 445 | 860 | 380 | 675 |
| FLAK 72 | 53 | 77 | > | > | > | > |
| FLAK 75 | 54 | 79 | > | > | > | > |
| SHIVA 10 (1-16) Ac | 55 | 80 | 28 | 64 | 97 | 28 |
| CA(1-7)Shiva10(1-16)-COOH | 56 | 81 | 8 | 22 | 19 | 170 |
| Indolocidin-ac | 57 | 91 | 9 | 64 | 20 | 31 |
| FLAK50B | 58 | 92 | 43 | 25 | 670 | 83 |
| FLAK50J | 59 | 93 | 530 | 320 | > | 690 |
| FLAK50I | 60 | 94 | 6 | ND | > | ND |
| FLAK50K | 61 | 95 | 250 | > | > | > |
| FLAK50L | 62 | 96 | > | > | > | > |
| Shiva-11 | 63 | 98 | 47 | 53 | 175 | 52 |
| SHIVA 11 | 64 | 99 | 34 | 54 | 180 | 28 |
| [(1-16)ME(2-9]-COOH | | | | | | |
| FLAK 50N | 65 | 101 | 300 | 340 | 170 | 730 |
| FLAK 50O | 66 | 102 | 73 | 27 | 43 | 66 |
| FLAK 50P | 67 | 103 | 26 | 150 | 70 | 330 |
| CA(1-&HECATE(11/23) | 68 | 104 | 24 | 52 | 130 | 18 |
| PYL-ME | 69 | 105 | 430 | > | > | ND |
| FLAG26-D1 | 70 | 106 | > | 920 | 700 | > |
| VISHNU3 | 71 | 107 | > | > | > | > |
| MELITTIIN | 72 | 108 | 16 | 25 | 35 | 13 |
| FLAK26-D2 | 73 | 109 | > | > | > | > |
| FLAG26-D3 | 74 | 110 | 45 | 95 | 540 | > |
| FLAK50 Q1 | 75 | 111 | 24 | 8 | 7 | 11 |
| FLAK50 Q2 | 76 | 112 | 420 | 470 | 660 | 640 |
| FLAK50 Q3 | 77 | 113 | 170 | 50 | 190 | 240 |
| FLAK50 Q4 | 78 | 114 | > | > | > | > |
| FLAK50 Q5 | 79 | 117 | > | > | > | > |
| FLAK50 Q6 | 80 | 118 | 170 | 74 | 87 | 330 |
| FLAK50 Q7 | 81 | 119 | 45 | 33 | 30 | 140 |
| FLAK50 Q8 | 82 | 120 | 600 | 620 | 810 | > |
| FLAK50 Q9 | 83 | 121 | 625 | 460 | 830 | > |
| FLAK50 Q10 | 84 | 122 | 720 | 830 | 780 | 800 |
| FLAK50 T1 | 85 | 123 | 600 | > | 940 | > |
| FLAK50 T2 | 86 | 124 | 21 | 30 | 14 | 10 |
| FLAK50 T3 | 87 | 125 | 90 | 76 | 220 | 145 |
| FLAK50 T4 | 88 | 126 | > | > | > | > |
| FLAK50 T5 | 89 | 127 | 760 | 770 | 610 | > |
| FLAK90 | 90 | 128 | 500 | > | 700 | > |
| FLAK91 | 91 | 129 | > | 790 | 550 | > |
| FLAK92 | 92 | 130 | > | > | > | > |
| FLAK93 | 93 | 131 | > | > | > | > |
| FLAK50 Z1 | 94 | 132 | > | > | > | > |
| FLAK50 Z2 | 95 | 133 | > | > | > | > |
| FLAK50 Z3 | 96 | 134 | > | > | > | > |
| FLAK50 Z4 | 97 | 135 | 110 | 115 | 215 | 310 |
| FLAK50 Z5 | 98 | 136 | > | 450 | 400 | 900 |
| FLAK50 Z6 | 99 | 137 | > | > | > | > |
| FLAK50 Z7 | 100 | 138 | > | > | > | > |
| FLAK50 Z8 | 101 | 139 | 550 | 850 | > | > |
| FLAK50 Z9 | 102 | 140 | > | > | 285 | > |
| FLAK94 | 103 | 141 | 420 | > | > | ND |
| FLAK93B | 104 | 142 | 73 | 115 | 55 | 60 |
| FLAK50 Z10 | 105 | 143 | > | > | > | > |
| FLAK96 | 106 | 144 | 750 | 225 | 275 | 350 |
| FLAK97 | 107 | 145 | > | > | 240 | > |
| FLAK98 | 108 | 146 | 270 | 93 | 640 | 440 |
| FKRLA | 109 | 147 | 83 | 93 | > | 340 |
| FLAK91B | 110 | 148 | 380 | 660 | > | > |
| FLAK92B | 111 | 149 | > | > | > | > |
| FLAK99 | 112 | 150 | 125 | 185 | 320 | 74 |
| FLAK50T6 | 113 | 151 | > | > | > | > |
| FLAK50T7 | 114 | 152 | 620 | 410 | > | > |
| FLAK95 | 115 | 153 | 130 | 50 | 140 | 97 |
| FLAK50T8 | 116 | 154 | 600 | 400 | > | 640 |
| FLAK50T9 | 117 | 155 | > | > | > | ND |
| FLAK100-CO2H | 118 | 156 | 230 | ND | > | 260 |
| FAGVL | 119 | 157 | 500 | 315 | > | 375 |
| Modelin-5 | 120 | 159 | 82 | 74 | 275 | 145 |
| Modelin-5-CO2H | 121 | 160 | 700 | 470 | 550 | 450 |
| FLAK120 | 126 | 165 | 470 | 56 | 400 | 340 |
| FLAK121 | 127 | 166 | > | > | > | > |
| FLAK96B | 128 | 167 | 260 | 300 | 325 | 320 |
| FLAK96G | 129 | 168 | > | > | > | > |
| FLAK96F | 130 | 169 | > | 640 | > | > |
| FLAK96C | 131 | 170 | > | > | > | > |
| FLAK96D | 132 | 171 | 615 | 540 | 820 | 600 |
| Modelin-8D | 135 | 174 | > | > | > | > |
| Modelin-8E | 136 | 175 | > | > | 510 | > |
| Flak 96H | 137 | 176 | > | > | > | > |
| Flak 961 | 138 | 177 | 270 | 240 | 380 | 120 |
| Flak 96J | 139 | 178 | 405 | > | > | > |
| Flak 96L | 140 | 179 | 540 | > | > | > |
| FLAK-120G | 141 | 180 | 940 | > | 760 | > |
| FLAK-120D | 142 | 181 | 500 | > | > | > |
| FLAK-120C | 143 | 182 | > | > | > | > |
| FLAK-120B | 144 | 183 | > | > | > | > |
| FLAK-120F | 145 | 184 | 800 | 370 | 302 | 570 |
| Magainin2wisc | 146 | 300 | 52 | 60 | 125 | 45 |
| D2A21 | 147 | 301 | 66 | 77 | 170 | 45 |
| KSL-1 | 148 | 302 | 800 | 720 | > | > |
| KSL-7 | 149 | 303 | 355 | 345 | > | 530 |
| LSB-37 | 150 | 306 | 320 | 120 | 250 | 370 |
| Anubis-2 | 151 | 307 | 75 | 160 | 100 | 66 |
| D-Shiva 10 AC | 163 | 100 | 95 | 220 | 150 | ND |
| Shiva 10 (1-18) AM | 164 | 69 | 101 | 71 | 190 | 81 |

Note:
> indicates greater than 1000;
ND indicates not determined;
numbers are in μg/mL.

It can be seen from Tables 16 and 17 that all targets challenged were inhibited by one or more of the peptides to an appreciable extent (i.e. LD50 less than 50 μg/ml). Table 18 below shows that 44 (29%) of the 150 peptides tested were active with some LD50 values at or below 50; 26 of the peptides were active on some targets at or below the LD50 value of 25; and 16 peptides were very active on one or more target strains with LD50 values at or below 10.

Table 19 below shows a broad spectrum of activity against six cancer cell types for various active peptides. It is noted that each target has one or more lead candidate peptides inhibitory to cell growth at an LD50 level of 10 or less.

TABLE 18
FLAK peptides showing substantial
activity against cancer cell lines

| | Number of | Percent of 150 |
|---|---|---|
| LD50 values | “active” peptides | peptides tested |
| < or = 50 μg/ml | 44 | 29% |
| < or = 25 μg/ml | 26 | 17% |
| < or = 10 μg/ml | 16 | 11% |

TABLE 19
Activity and specificity of FLAK peptides against six cancer cell targets

| | Number of active peptides per target | | | | | |
|---|---|---|---|---|---|---|
| | MCF7 | SW480 | BMKC | H1299 | HeLaS3 | PC3 |
| LD50 | (breast) | (colon) | (melanoma) | (lung) | (cervix) | (prostate) |
| < or = 50 μg/ml | 31 | 25 | 19 | 19 | 17 | 20 |
| < or = 25 μg/ml | 17 | 13 | 8 | 10 | 8 | 11 |
| < or = 10 μg/ml | 6 | 5 | 3 | 4 | 1 | 5 |

### Example 5

### Stimulation and Proliferation of Leukocytes

In vitro viability of human leukocyte cells in the presence of different peptides at different concentrations was determined by an Alamar Blue protocol. Alamar Blue (Promega, Madison, Wis.) is an indicator dye, formulated to measure quantitatively the proliferation and cytotoxicity of the cells. The dye consists of an oxidation-reduction (redox) indicator that yields a calorimetric change and a fluorescent signal in response to cellular metabolic activity.

Assay protocol: Blood from a 50 year old male human was drawn and centrifuged at 1500 rpm for 15 minutes at room temperature. The buffy coat cells at the plasma-red blood cell interface were aspirated. Buffy coat cells (mainly lymphocyte cells) were then transferred into 15 ml centrifuge tubes containing 5 ml of RPMI-1640 medium+10% Fetal Bovine Serum (Gibco, Grand Island, N.Y.). Additional medium was added to the tubes to bring the volume up to 10 ml. The buffy coat suspension was then carefully layered on 5 ml of Histopaque (Sigma Chemical Co., St. Louis, Mo.) and centrifuged at 1500 rpm for 30 minutes at room temperature. The interface which is mostly PBMCs (peripheral mononuclear cells) was aspirated and transferred to a 15 ml conical centrifuge tube and, resuspended in 2 ml cold RPMI-1640 and brought up to 15 ml with cold RPMI-1640 medium. Cells were centrifuged at 1500 rpm for 10 minutes. The supernatant was then aspirated and discarded. The cell pellet was re-suspended in 1 ml of cold RPMI 1640 and brought up to 15 ml with RPMI medium. This step was repeated twice, except that in the last step, the cells were resuspended with 1 ml of cold RPMI-1640 medium and cell counts were performed with a hemocytometer according to the Sigma cell culture catalogue.

Pokewood mitogen was used as a control along with positive and negative controls. Negative control cells were killed with 70% methanol. Positive (+) control cells were incubated in RPMI medium (untreated). 20 ml of AlamarBlue was added to the cells, and readings were taken after 24 hours, 48 hours, 72 hours, and 96 hours using a fluorimeter (excitation 544/transmission 590 nm).

Calculations were performed using the following formula. The peptide treated sample and positive control were adjusted for negative control.

$$\text{\% treated cell stimulation/proliferation} = \frac{\text{Peptide treated sample}}{\text{Positive control}} \times 100 \%$$

Using the protocol described immediately above, about 100-150 peptides were screened for their stimulatory and/or inhibitory actions upon the growth of human leukocyte (“WBC”) cells as compared to the growth of untreated positive control cells. The data in Table 20 below show that various selected FLAK peptides are stimulatory at low concentrations (0.1 to 1.0 μg/ml), whereas certain of the peptides become inhibitory (causing cell death) at higher concentrations. Several of the peptides (i.e. SEQ ID NOS: 5, 143, and 160) are stimulatory (and/or proliferative) at all concentrations through 500 μg/ml.

The Alamar Blue stain used in the protocol permeates both cell and nuclear membranes, and is metabolized in the mitochondria to cause the change in color. The resulting fluorometric response is therefore a result of total mitochondrial activity caused by cell stimulation and/or mitosis (cell proliferation). The increase in values (for treated cells, as a percent of values for untreated cells) with increased incubation time (120 hours vs. 48 hours) may be attributed to increased cell proliferation in addition to stimulation of cell metabolic activity caused by the peptide.

Table 20 presents peptide treated cell stimulation/proliferation, as percent of untreated positive control, for human leukocytes (white blood cells, “WBC”) in the presence of selected FLAK peptides. The table also shows for each of these peptides its toxicity (LD50 values) to human red blood cells (RBC) and to human fibroblast cells (WI38). Those certain peptides which are stimulatory to WBCs at low peptide concentrations (i.e. 10 μg/ml or less) and are inhibitory or toxic to WBCs at higher concentrations are also relatively more toxic to RBCs and to fibroblasts than those peptides which are stimulatory and not inhibitory to WBC growth even at concentrations as high as 500 μg/ml.

In limited experiments with other than the Alamar Blue protocol described above, it has been qualitatively determined that those peptides which cause stimulation and proliferation of leukocytes are active upon both the phagocytic and lymphocyte cell components of the mammalian lymphatic system. As such, certain of the stimulatory FLAK peptides which are relatively non-toxic to mammalian cells at therapeutic dose levels may be used as immunomodulators to treat humans or other mammals with compromised immune systems. Such treatment may be administered systemically in vivo or by extra-corporeal treatment of whole blood or blood components to be reinfused to the donor. Such therapy would serve to counteract immune deficiency in neutropenic patients caused by age, disease, or chemotherapy and would stimulate natural immune responses to prevent or combat pathogenic infections and growth of certain cancer cell lines or to enhance wound healing processes involving the lymphoid system. Table 21 is a more detailed example (with one peptide, SEQ ID NO:10) of the phenomenon showing the relationships of concentration and time as they effect stimulation, proliferation, and inhibition of the leukocytes.

TABLE 20
Human lymphocyte (WBC) stimulation/proliferation
by selected FLAK peptides

| Selected | | Peptide treated cell activity | | | | | Peptide | |
|---|---|---|---|---|---|---|---|---|
| peptides | | Percent stimulation relative to control | | | | | toxicity | |
| SEQ | P | 0.1 | 1 | 10 | 100 | 500 | RBC | WI-38 |
| ID NO. | NO. | ug/ml | ug/ml | ug/ml | ug/ml | ug/ml | LD/50 | LD/50 |
| 2 | 2 | 117 | 118 | 119 | 121 | 119 | 30 | 66 |
| 5* | 12 | 111 | 115 | 118 | 116 | 101 | >1000 | 950 |
| 10 | 25 | 117 | 104 | 99 | 27 | 27 | 60 | 24 |
| 12 | 27 | 108 | 110 | 99 | 30 | 23 | 125 | 37 |
| 17 | 35 | 82 | 76 | 61 | 18 | 16 | 200 | 8 |
| 20 | 38 | 79 | 82 | 78 | 37 | 36 | 350 | 12 |
| 25 | 43 | 78 | 82 | 71 | 14 | 12 | 20 | 5 |
| 30 | 48 | 74 | 68 | 62 | 13 | 13 | 130 | 60 |
| 58 | 92 | 112 | 112 | 98 | 35 | 26 | 300 | 25 |
| 61 | 95 | 110 | 115 | 116 | 124 | 114 | >1000 | >1000 |
| 165 | 97 | 107 | 109 | 106 | 27 | 22 | 350 | 85O |
| 66 | 102 | 100 | 102 | 97 | 37 | 17 | 500 | 210 |
| 71 | 107 | 101 | 100 | 108 | 109 | 110 | >1000 | >1000 |
| 115 | 153 | 93 | 92 | 37 | 72 | 29 | 780 | 130 |
| 119* | 157 | 88 | 108 | 54 | 117 | 89 | 850 | 500 |
| 147* | 301 | 100 | 94 | 83 | 22 | 20 | 10 | 66 |
| 150* | 306 | 97 | 101 | 94 | 109 | 112 | >1000 | 320 |

*not a FLAK peptide;
incubation times were 48 hours for all samples

TABLE 21
Human leukocyte (WBC) stimulation/proliferation and
inhibition by FLAK peptide SEQ ID NO: 10 (P25)

| | | | | | |
|---|---|---|---|---|---|
| Time of | 0.1 | 1 | 10 | 100 | 500 |
| incubation | μg/ml | μg/ml | μg/ml | μg/ml | μg/ml |

| | | | | | | |
|---|---|---|---|---|---|---|
| 24 | hours | 111 | 98 | 88 | 10 | 10 |
| 48 | hours | 117 | 104 | 99 | 27 | 27 |
| 72 | hours | 119 | 105 | 102 | 31 | 32 |
| 96 | hours | 128 | 112 | 110 | 38 | 40 |
| 120 | hours | 135 | 118 | 119 | 43 | 45 |

Note:
Number values are percent peptide treated cell stimulation/proliferation relative to control cells (100%)

### Example 6

### Stimulation and Proliferation of Fibroblasts

The cyQUANT cell proliferation assay provides a convenient, rapid and sensitive procedure for determining the density of cells in culture. The assay has a linear detection range extending from 50 or fewer to at least 50,000 cells in 200 μl volumes using a single dye concentration. The assay is ideal for cell proliferation studies as well as for routine cell counts and can be used to monitor the adherence of cells to surfaces.

Procedure: Different cell lines were maintained with different medium according to the ATCC. Cells were trypsinized with 8 ml of Trypsin (0.25%, Fisher, Pittsburgh, Pa.). The cell suspension was centrifuged for 10 minutes at 100 rpm. The supernatant was removed and discarded without disturbing the cell pellet. A concentrated cell suspension was prepared in 1.0 ml of medium to obtain a density of about 10⁵ to 10⁶ cells/ml. The actual cell density was determined by counting the cells using a hemocytometer with the Trypan Blue method. Cell numbers were adjusted to obtain equal number of cells per 200 μl volume. Cells were plated with 0% FBS, 2% FBS, 3% FBS and 5% FBS. The plates were incubated at 37° C. for a time sufficient to allow the cells to attach. For long-term proliferation studies, 100 μl of medium was removed from each well each day and replaced with fresh medium.

At the desired time, the medium was removed from the adherent cells in a 96 well plate. These cells were already treated with test agents. The cells were frozen in the plate at −70° C. for 30 minutes. The cells were thawed at room temperature. CyQuant GR dry/Cell Lysis Buffer (200 μl) was added to each sample cell. The cells were incubated at room temperature for 15 minutes while protected from the light. Fluorescence was measured using fmax at 485-538 nm.

The above CyQuant protocol was used to examine possible peptide stimulation and/or proliferation of fibroblasts. In the following Table 22, data are shown for selected peptides demonstrating their effect on human fibroblast cells (WI38). In the table, the substantial stimulatory and/or proliferative property of selected peptides, as a function of concentration is evident. Table 23 shows that the fibroblast stimulation and/or proliferation effect is enhanced for certain peptides in the presence of other growth factors. This is shown by the addition of Fetal Bovine Serum (FBS) to the medium. Number values shown in Tables 22 and 23 are cell stimulation/proliferation activity expressed as a percent of control (untreated cells). Control cells and peptide treated cells are with medium and FBS as indicated. Values below 100% (for control) indicate inhibitory action of the peptide, especially at concentrations above 10 μg/ml.

TABLE 22
Human fibroblast (WI-38) cell stimulation
by selected FLAK peptides

| | | | | Peptide treated cell activity | | | |
|---|---|---|---|---|---|---|---|
| | | | | Stimulation relative to control | | | |
| SEQ | | Inc. Time | % FBS in | 0.1 | 1 | 10 | 100 |
| ID NO: | P No. | (hrs)** | serum | μg/ml | μg/ml | μg/ml | μg/ml |
| 2 | 2 | 48 | 2.0 | 125 | 156 | 122 | 35 |
| 4 | 11 | 48 | 2.0 | 149 | 145 | 166 | 113 |
| 5* | 12 | 48 | 3.0 | 111 | 116 | 109 | 120 |
| 10 | 25 | 48 | 2.0 | 137 | 143 | 120 | 73 |
| 12 | 27 | 48 | 2.0 | 134 | 115 | 104 | 116 |
| 25 | 43 | 48 | 3.0 | 93 | 99 | 83 | 14 |
| 30 | 48 | 48 | 3.0 | 117 | 117 | 109 | 110 |
| | | 72 | 3.0 | 119 | 123 | 139 | 144 |
| 32 | 50 | 72 | 3.0 | 108 | 123 | 127 | 56 |
| 35 | 55 | 48 | 3.0 | 101 | 109 | 116 | 25 |
| | | 72 | 3.0 | 91 | 98 | 101 | 6 |
| 61 | 95 | 72 | 3.0 | 101 | 90 | 94 | 93 |
| 66 | 102 | 72 | 3.0 | 123 | 121 | 126 | 122 |
| 71* | 107 | 72 | 3.0 | 114 | 104 | 98 | 86 |
| 80 | 118 | 72 | 3.0 | 163 | 193 | 192 | 184 |
| 108 | 146 | 72 | 3.0 | 109 | 101 | 84 | 74 |
| 115 | 153 | 72 | 3.0 | 125 | 125 | 132 | 106 |
| 119* | 157 | 72 | 3.0 | 126 | 118 | 104 | 119 |
| 126 | 165 | 72 | 3.0 | 133 | 119 | 79 | 129 |
| 147* | 301 | 48 | 3.0 | 87 | 98 | 95 | 58 |
| 150* | 306 | 48 | 3.0 | 102 | 103 | 101 | 94 |

*not a FLAK peptide;
**incubation time in hours.

TABLE 23
Effect of growth factors on human
fibroblast (WI38) cell stimulation

| | | | Peptide concentration | | | |
|---|---|---|---|---|---|---|
| SEQ ID | | % FBS in | 0.1 | 1 | 10 | 100 |
| NO: | P Number | serum | μg/ml | μg/ml | μg/ml | μg/ml |
| 2 | 2 | 0 | −27 | −3 | 27 | −82 |
| | | 2.5 | 26 | 57 | 23 | −66 |
| 4 | 11 | 0 | 19 | 34 | 50 | −40 |
| | | 2.5 | 50 | 52 | 62 | 14 |
| 8 | 23 | 0 | 21 | 78 | 10 | −48 |
| | | 2.5 | 16 | 23 | 58 | 75 |
| 80 | 118 | 0 | 12 | −4 | −7 | −1 |
| | | 3 | 61 | 70 | 68 | 72 |

Note:
Number values are percent cell viability above or below control.

### Example 7

### Toxicity Assay—Red Blood Cell (RBC) Hemolysis, and Leukocyte (WBC) and Fibroblast (WI38) Inhibition

Table 24 below summarizes the RBC, WBC, and WI38 toxicity data for typical FLAK peptides. The three RBC, WBC, and WI38 values (LD50) are generally consistent directional indicators of peptide toxicity. In choosing a peptide for possible treatment of a given indication it is important to match the therapeutic activity and specificity of the peptide with its possible toxic properties. The SEQ ID NO:5 peptide is not a FLAK peptide, but rather it is SB-37, a close homolog of Cecropin B. It has previously been shown not to be as active as the FLAK peptides as an antibacterial agent, but to possess wound healing properties as demonstrated in vivo in a rat model. This probably results from its stimulatory and proliferative effects on both mammalian leukocytes and fibroblasts.

The protocols for WBC and WI38 stimulation have been discussed above. The RBC protocol follows Table 24.

TABLE 24
In vitro toxicity of selected FLAK peptides on red blood cells
(RBC), human leukocytes (WBC), and human fibroblasts (WI38)

| | | RBC LD50 | WBC LD50 | WI38 LD50 |
|---|---|---|---|---|
| SEQ ID NO: | P Number | μg/ml | μg/ml | μg/ml |
| 5 | 12 | >1000 | >500 | 60 |
| 10 | 25 | 60 | 79 | 60 |
| 11 | 26 | 900 | 185 | 100 |
| 12 | 27 | 125 | 78 | 60 |
| 16 | 34 | 200 | 77 | 200 |
| 17 | 35 | 200 | 64 | 25 |
| 20 | 38 | 350 | 160 | 100 |
| 25 | 43 | 20 | 70 | 25 |
| 30 | 48 | 130 | 78 | 70 |
| 35 | 55 | 30 | 80 | 28 |
| 58 | 92 | 300 | 51 | 400 |
| 66 | 102 | 300 | 115 | 45 |

The RBC protocol is as follows. Well positions of each dilution and untreated controls are recorded on the lid of a 96-well plate. When the cells were confluent, the media is removed, and replaced with freshly prepared sample dilutions to a final volume of 200 μl. Test agent was added into designed wells of the 96-well plate. The 200 μl fresh medium was added to positive control wells; and 200 μl of 70% ethanol was added to negative control wells. The plate was incubated overnight at 37° C., 5% CO₂, and at least 90% humidity. Room temperature AlamarBlue solution (20 μl) was added to all wells. The plates were read spectrofluorometrically (excitation 544 nm, emission 590 nm). The plates were incubated for 3 hours at 37° C., 5% CO₂, and at least 90% humidity. The plates were read again at 3 and 24 hours incubation. The LD50 endpoint was determined from the graph by reading from where the 50 percent point intercepts the Dose Response Curve to the concentration along the x-axis. That concentration is the LD50 value. The LD50 value for test agents within a single test agent class can be used to rank-order their relative toxicities or to correlate with in vivo data.

This hemolytic assay is based upon that presented in Journal of Peptide Research 53: 82-90 (1999). Preparation of all media, stock solutions and dilutions were performed in a laminar flow hood to minimize or prevent contamination. All procedures were performed according to safety protocols pertaining to the handling and disposal of human body fluids.

Red blood cells (RBCs) were washed three times with PBS (35 mM phosphate buffer 0.15 M NaCl, pH 7.0). RBCs suspended in PBS (0.4% (v/v); about 10 ml per 15 peptides) were prepared. Suspensions (100 μl) were aliquoted to each sample and control tube. Serially diluted peptide solutions (100 μl) were pipetted into the sample tubes. Negative control tubes contained 100 μl PBS; positive control tubes contained 100 μl 1% Triton-X100 detergent. All tubes were incubated for 1 hour at 37° C. The tubes were removed from the incubator and centrifuged at 1000 g for 5 minutes. Supernatant (100 μl) was pipetted to a 96-well polyvinyl chloride plate. The absorbance at 414 nm (A₄₁₄) was measured, and used to calculate the percent hemolysis according to the following formula.

$$\frac{( {A}_{414} in peptide solution - {A}_{414} in PBS )}{( {A}_{414} in \text{Triton} \text{-} X 100 - {A}_{414} in PBS )} \times 100 \%$$

Percent hemolysis is plotted against peptide concentration, and the concentration at which 50% hemolysis is determined (LD₅₀). The following Table 25 details the results of the hemolytic assay using the peptides discussed herein.

TABLE 25

| | SEQ ID | | LD₅₀ |
|---|---|---|---|
| Peptide name | NO: | P Number | μg/mL |
| Hecate AC #1010 | 1 | 1 | 100 |
| Hecate AM | 2 | 2 | 10 |
| SB-37 AC #1018 | 3 | 5 | > |
| Shiva 10 AM | 4 | 11 | 76 |
| SB-37 AM | 5 | 12 | > |
| Shiva 10 AC #1015 | 6 | 13 | 50 |
| Magainin 2 | 7 | 16 | 550 |
| FLAK01 AM | 8 | 23 | 300 |
| FLAK03 AM | 9 | 24 | 10 |
| FLAK04 AM | 10 | 25 | 16 |
| FLAK05 AM | 11 | 26 | 90 |
| FLAK06 AM | 12 | 27 | 125 |
| FLAK06 AC | 13 | ^{27B} | 700 |
| FLAK06 R-AC | 14 | ^{27C} | 250 |
| KALV | 15 | 30 | 150 |
| FLAK 17 AM | 16 | 34 | 200 |
| FLAK 26 AM | 17 | 35 | 200 |
| FLAK 25 AM | 18 | 36 | 85 |
| Hecate 2DAc | 19 | 37 | 30 |
| FLAK43 AM | 20 | 38 | 350 |
| FLAK44 AM | 21 | 39 | > |
| FLAK62 AM | 22 | 40 | > |
| FLAK 06R-AM | 23 | 41 | 40 |
| MSI-78 AM | 24 | 42 | 300 |
| FLAK50 | 25 | 43 | 20 |
| FLAK51 | 26 | 44 | 90 |
| FLAK57 | 27 | 45 | 700 |
| FLAK71 | 28 | 46 | 900 |
| FLAK77 | 29 | 47 | > |
| FLAK50V | 30 | 48 | 200 |
| FLAK50F | 31 | 49 | 225 |
| FLAK26V AM | 32 | 50 | 420 |
| CAME-15 | 33 | 53 | 20 |
| FLAK50C | 34 | 54 | 250 |
| FLAK50D | 35 | 55 | 20 |
| FLAK 50E | 36 | 56 | 600 |
| FLAK80 | 37 | 57 | > |
| FLAK81 | 38 | 58 | > |
| FLAK82 | 39 | 59 | 1000 |
| FLAK83M | 40 | 60 | > |
| FLAK 26 Ac | 41 | 61 | 390 |
| Indolicidin | 42 | 63 | 375 |
| FLAK 17 C | 43 | 64 | 6 |
| FLAK 50H | 44 | 65 | 950 |
| FLAK 50G | 45 | 66 | 600 |
| Shiva deriv P69 + KWKL | 46 | 70 | 80 |
| Shiva 10(1-18_AC | 47 | 71 | > |
| Shiva 10 peptide 71 + KWKL | 48 | 72 | 110 |
| CA(1-7)Shiva10(1-16) | 49 | 73 | 90 |
| FLAK 54 | 50 | 74 | > |
| FLAK 56 | 51 | 75 | 750 |
| FLAK 58 | 52 | 76 | > |
| FLAK 72 | 53 | 77 | > |
| FLAK 75 | 54 | 79 | > |
| Shiva 10 (1-16) Ac | 55 | 80 | 900 |
| CA(1-7)Shiva10(1-16)-COOH | 56 | 81 | 8 |
| Indolocidin-ac | 57 | 91 | 40 |
| FLAK50B | 58 | 92 | 300 |
| FLAK50J | 59 | 93 | > |
| FLAK50I | 60 | 94 | 350 |
| FLAK50K | 61 | 95 | > |
| FLAK50L | 62 | 96 | > |
| Shiva-11 | 63 | 98 | 60 |
| Shiva 11[(1-16)ME(2-9)]-COOH | 64 | 99 | 25 |
| FLAK 50N | 65 | 101 | 550 |
| FLAK 50O | 66 | 102 | 500 |
| FLAK 50P | 67 | 103 | 650 |
| CA(1-&Hecate(11/23) | 68 | 104 | 70 |
| PYL-ME | 69 | 105 | ND |
| FLAG26-D1 | 70 | 106 | > |
| Vishnu3 | 71 | 107 | > |
| Melittin | 72 | 108 | <1 |
| FLAK26-D2 | 73 | 109 | > |
| FLAG26-D3 | 74 | 110 | > |
| FLAK50 Q1 | 75 | 111 | 60 |
| FLAK50 Q2 | 76 | 112 | > |
| FLAK50 Q3 | 77 | 113 | 1000 |
| FLAK50 Q4 | 78 | 114 | > |
| FLAK50 Q5 | 79 | 117 | > |
| FLAK50 Q6 | 80 | 118 | 700 |
| FLAK50 Q7 | 81 | 119 | 400 |
| FLAK50 Q8 | 82 | 120 | > |
| FLAK50 Q9 | 83 | 121 | > |
| FLAK50 Q10 | 84 | 122 | > |
| FLAK50 T1 | 85 | 123 | 1000 |
| FLAK50 T2 | 86 | 124 | 55 |
| FLAK50 T3 | 87 | 125 | > |
| FLAK50 T4 | 88 | 126 | > |
| FLAK50 T5 | 89 | 127 | > |
| FLAK90 | 90 | 128 | > |
| FLAK91 | 91 | 129 | > |
| FLAK92 | 92 | 130 | > |
| FLAK93 | 93 | 131 | > |
| FLAK50 Z1 | 94 | 132 | > |
| FLAK50 Z2 | 95 | 133 | > |
| FLAK50 Z3 | 96 | 134 | > |
| FLAK50 Z4 | 97 | 135 | 900 |
| FLAK50 Z5 | 98 | 136 | > |
| FLAK50 Z6 | 99 | 137 | > |
| FLAK50 Z7 | 100 | 138 | 20 |
| FLAK50 Z8 | 101 | 139 | > |
| FLAK50 Z9 | 102 | 140 | > |
| FLAK94 | 103 | 141 | 900 |
| FLAK93B | 104 | 142 | 900 |
| FLAK50 Z10 | 105 | 143 | > |
| FLAK96 | 106 | 144 | 600 |
| FLAK97 | 107 | 145 | > |
| FLAK98 | 108 | 146 | 180 |
| FKRLA | 109 | 147 | 300 |
| FLAK91B | 110 | 148 | > |
| FLAK92B | 111 | 149 | > |
| FLAK99 | 112 | 150 | 650 |
| FLAK50T6 | 113 | 151 | > |
| FLAK50T7 | 114 | 152 | 880 |
| FLAK95 | 115 | 153 | 800 |
| FLAK50T8 | 116 | 154 | 450 |
| FLAK50T9 | 117 | 155 | > |
| FLAK100-CO2H | 118 | 156 | 10 |
| FAGVL | 119 | 157 | 850 |
| Modelin-5 | 120 | 159 | ND |
| Modelin-5-CO2H | 121 | 160 | > |
| FLAK120 | 126 | 165 | 350 |
| FLAK121 | 127 | 166 | > |
| FLAK96B | 128 | 167 | 200 |
| FLAK96G | 129 | 168 | 600 |
| FLAK96F | 130 | 169 | > |
| FLAK96C | 131 | 170 | > |
| FLAK96D | 132 | 171 | 550 |
| Modelin-8D | 135 | 174 | > |
| Modelin-8E | 136 | 175 | > |
| Flak 96 | 137 | 176 | > |
| Flak 96I | 138 | 177 | 400 |
| Flak 96J | 139 | 178 | > |
| Flak 96L | 140 | 179 | 850 |
| FLAK-120G | 141 | 180 | > |
| FLAK-120D | 142 | 181 | > |
| FLAK-120C | 143 | 182 | > |
| FLAK-120B | 144 | 183 | > |
| FLAK-120F | 145 | 184 | 850 |
| Magainin2wisc | 146 | 300 | 250 |
| D2A21 | 147 | 301 | 10 |
| KSL-1 | 148 | 302 | > |
| KSL-7 | 149 | 303 | 500 |
| LSB-37 | 150 | 306 | > |
| Anubis-2 | 151 | 307 | > |
| FLAK17CV | 152 | 501 | 15 |
| FLAK50Q1V | 153 | 502 | 100 |
| D2A21V | 154 | 503 | 20 |
| FLAK25AMV | 155 | 504 | 70 |
| FLAK43AMV | 156 | 505 | 620 |
| FLAK50DV | 157 | 506 | 120 |
| HECATE AMV | 158 | 507 | 20 |
| HECATE ACV | 159 | 508 | 70 |
| FLAK04AMV | 160 | 509 | 40 |
| FLAK03AMV | 161 | 510 | 10 |
| D-Shiva 10 AC | 162 | 67 | 40 |
| Shiva 11 AC | 163 | 100 | > |
| Shiva 10 (1-18) AM | 164 | 69 | 900 |

Note:
> indicates greater than 1000;
ND = not determined.

### Example 8

### Effects of Valine Substitution

Changing a peptide sequence where the first amino acid is valine, and particularly when the first amino acid is changed from phenylalanine to valine, can lead to desirable properties. The red blood cell and fibroblast cell (WI38) toxicity can be decreased, while not significantly decreasing other desirable properties. Table 26 below shows numerous examples (14) of reducing the indicated toxicity of a peptide as seen from increase in viability of both red blood cells and fibroblast cells when treated with peptide. LD50 values are in μg/ml.

TABLE 26

SEQ.

| | | | |
|---|---|---|---|
| ID | P | Hemolysis | WI-38 |

| | | | | |
|---|---|---|---|---|
| NO: | No. | Sequence | RBC LD50 | LD50 |

| | | | | |
|---|---|---|---|---|
| 2 | 2 | FALALKALKKALKKLKKALKKAL-NH2 | 12 | 66 |
| 15 | 30 | VALALKALKKALKKLKKALKKAL-NH2 | 150 | 93 |
| 17 | 35 | FAKKLAKLAKKLAKLAL-NH2 | 150 | 25 |
| 32 | 50 | VAKKLAKLAKKLAKLAL-NH2 | 420 | 45 |
| 25 | 43 | FAKLLAKLAKKLL-NH2 | 20 | 25 |
| 30 | 48 | VAKLLAKLAKKLL-NH2 | 130 | 160 |
| 86 | 124 | FAKLLAKLAKKVL-NH2 | 55 | 21 |
| 116 | 154 | VAKLLAKLAKKVL-NH2 | 870 | 110 |
| 126 | 165 | FALALKALKKL-NH2 | 350 | 850 |
| 141 | 180 | VALALKALKKL-NH2 | 850 | 1000 |
| 43 | 64 | FAKALKALLKALKAL-NH2 | 6 | 37 |
| 152 | 501 | VAKALKALLKALKAL-NH2 | 15 | 26 |
| 75 | 111 | FAKFLAKFLKKAL-NH2 | 5 | 25 |
| 153 | 502 | VAKFLAKFLKKAL-NH2 | 100 | 64 |
| 147 | 301 | FAKKFAKKFKKFAKKFAKFAFAF-NH2 | 10 | 66 |
| 154 | 503 | VAKKFAKKFKKFAKKFAKFAFAF-NH2 | 20 | 150 |
| 18 | 36 | FAKKLAKLAKKLAKLALAL-NH2 | 12 | 19 |
| 155 | 504 | VAKKLAKLAKKLAKLALAL-NH2 | 70 | 110 |
| 20 | 38 | FAKKLAKLAKKLLAL-NH2 | 350 | 100 |
| 156 | 505 | VAKKLAKLAKKLLAL-NH2 | 620 | 85 |
| 35 | 55 | FAKLLAKALKKLL-NH2 | 20 | 32 |
| 157 | 506 | VAKLLAKALKKLL-NH2 | 120 | 75 |
| 1 | 1 | FALALKALKKALKKLKKALKKAL-COOH | 20 | 27 |
| 159 | 508 | VALALKALKKALKKLKKALKKAL-COOH | 70 | 190 |
| 10 | 25 | FALALKALKKLAKKLKKLAKKAL-NH2 | 16 | 24 |
| 160 | 509 | VALALKALKKLAKKLKKLAKKAL-NH2 | 40 | 95 |
| 9 | 24 | FALALKALKKLLKKLKKLAKKAL-NH2 | 10 | 55 |
| 161 | 510 | VALALKALKKLLKKLKKLAKKAL-NH2 | 10 | 77 |

Although the effects of reduction of toxicity to mammalian cells by valine substitution is accompanied by modest reductions of therapeutic activity against microbial pathogens and cancer cells, there are some cases in which the valine substitution results in a desirable increase in therapeutic activity. This can be seen in the following Table 27 where it is shown that the valine substitution in some cases has increased the peptide's activity against the gram negative bacterium Pseudomonas.

Hemolysis and WI38 values represent LD50 values. P. aerug values represent MIC values in μg/mL against Pseudomonas aeruginosa ATCC accession number 9027.

TABLE 27

SEQ
ID

| | | | | | |
|---|---|---|---|---|---|
| NO: | P No. | Sequence | Hemolysis | WI38 | P. aerug |

| | | | | | |
|---|---|---|---|---|---|
| 17 | 35 | FAKKLAKLAKKLAKLAL | 100 | 25 | 200 |
| 32 | 50 | VAKKLAKLAKKLAKLAL | 420 | 45 | 15 |
| 25 | 43 | FAKLLAKLAKKLL | 20 | 25 | 100 |
| 30 | 48 | VAKLLAKLAKKLL | 200 | 160 | 5 |
| 86 | 124 | FAKLLAKLAKKVL | 300 | 21 | 100 |
| 116 | 154 | VAKLLAKLAKKVL | 450 | 110 | 100 |

### Example 9

### Effects of Tyrosine Substitution

Changing a peptide sequence where the second amino acid is tyrosine can lead to desirable properties. FLAK98 (P-146, SEQ ID NO:108) is an atypical FLAK peptide due to the presence of a tyrosine (Y) at the second position. The significance of this modification and the peptide's overall sequence is that the structure produced is likely to fold readily into an alpha-helix at neutral pH (Montserret et al., Biochemistry 39: 8362-8373, 2000). The ability to assume an alpha-helical structure at neutral pH may account for the potency and broad spectrum of activity seen with this peptide. Montserret et al. demonstrated that sequences such as these are driven into folding not only by hydrophobic but also by electrostatic forces. The substitution of tyrosine for an amino acid in FLAK peptides may generally lead to improved properties.

### Example 10

### Presently Preferred Peptides

Preferred peptides can be selected from the above described experimental data. Preferred antimicrobial peptides for gram positive or gram negative bacteria can be selected as having MIC values of less than or equal to about 10 μg/ml, or as having MBC values of less than or equal to about 25 μg/ml. Preferred antifungal peptides can be selected as having MIC or MBC values of less than or equal to about 25 μg/ml. Preferred anticancer peptides can be selected as having LD50 values of less than or equal to about 25 μg/ml.

The following Table 28 lists representative presently preferred peptides, where an ‘X’ indicates that the peptide is a preferred peptide for that column's property. The peptide's “length” is the number of amino acid residues in the sequence.

TABLE 28

| SEQ ID | | Length | Anti- | Anti- | Anti- |
|---|---|---|---|---|---|
| NO: | P-number | (AA) | bacterial | fungal | cancer |
| 1 | 1 | 23 | X | | X |
| 2 | 2 | 23 | X | X | X |
| 4 | 11 | 23 | X | | |
| 6 | 13 | 23 | X | | |
| 8 | 23 | 23 | X | | X |
| 10 | 25 | 23 | X | X | |
| 11 | 26 | 21 | X | X | X |
| 12 | 27 | 19 | X | X | |
| 13 | 27B | 19 | X | X | X |
| 14 | 27C | 19 | X | | |
| 15 | 30 | 23 | X | | |
| 16 | 34 | 16 | X | X | X |
| 17 | 35 | 17 | X | X | X |
| 18 | 36 | 19 | X | | X |
| 19 | 37 | 23 | X | | X |
| 20 | 38 | 15 | X | | X |
| 23 | 41 | 19 | X | | |
| 25 | 43 | 13 | X | X | X |
| 26 | 44 | 15 | X | | X |
| 27 | 45 | 14 | X | | |
| 28 | 46 | 15 | X | | |
| 29 | 47 | 12 | | | X |
| 30 | 48 | 13 | X | X | X |
| 31 | 49 | 12 | X | | |
| 32 | 50 | 17 | X | | X |
| 34 | 54 | 13 | X | | |
| 35 | 55 | 13 | X | X | X |
| 36 | 56 | 13 | X | | |
| 39 | 59 | 10 | X | | |
| 41 | 61 | 15 | X | | |
| 43 | 64 | 15 | X | | |
| 45 | 66 | 13 | X | | |
| 46 | 70 | 23 | X | | X |
| 47 | 71 | 18 | X | | |
| 48 | 72 | 22 | X | | |
| 50 | 74 | 13 | | X | |
| 51 | 75 | 13 | X | | X |
| 52 | 76 | 14 | X | | |
| 55 | 80 | 23 | X | | |
| 56 | 81 | 23 | X | | X |
| 57 | 91 | 15 | X | | X |
| 58 | 92 | 13 | X | X | X |
| 60 | 94 | 13 | X | | X |
| 65 | 101 | 13 | X | | |
| 66 | 102 | 13 | X | X | |
| 67 | 103 | 12 | X | X | |
| 68 | 104 | 20 | X | | X |
| 74 | 110 | 12 | X | | |
| 75 | 111 | 13 | X | | X |
| 77 | 113 | 13 | X | | |
| 80 | 118 | 13 | X | X | |
| 81 | 119 | 14 | X | X | |
| 84 | 122 | 13 | X | X | |
| 85 | 123 | 10 | | X | |
| 86 | 124 | 13 | X | X | X |
| 87 | 125 | 13 | X | | |
| 93 | 131 | 5 | X | | |
| 106 | 144 | 12 | X | X | |
| 108 | 146 | 13 | X | X | |
| 112 | 150 | 17 | X | | |
| 115 | 153 | 17 | X | X | |
| 116 | 154 | 13 | | X | |
| 126 | 165 | 11 | X | X | |
| 128 | 167 | 12 | X | X | |
| 131 | 170 | 10 | | X | |
| 143 | 182 | 10 | | X | |
| 152 | 501 | 15 | X | | X |
| 155 | 504 | 13 | X | | |
| 157 | 506 | 23 | X | | X |
| 161 | 510 | 23 | X | X | |
| 162 | 67 | 23 | X | | X |
| 163 | 100 | 13 | X | X | |
| 164 | 69 | 23 | X | | |
| 165 | 97 | 13 | X | X | |

Preferred peptides for stimulation and proliferation can also be selected. The following Table 29 lists representative preferred peptides, where an ‘X’ indicates that the peptide is a preferred peptide for that column's property. Peptides which are stimulatory for leukocytes at 0.1 μg/ml to 1.0 μg/ml concentration are preferred, as at this concentration the peptides are not toxic to red blood cells, WI-38 fibroblasts, or to human leukocytes. Peptides which are stimulatory for fibroblasts at 0.1 μg/ml to 1.0 μg/ml are preferred as at this concentration the peptides are not toxic.

In Table 29 μlease add peptides P146 (SEQ 108) (Length=13) and P97 (SEQ 165) (Length=13). Both of these peptides should have X in the Leukocyte and in the Fibroblast columns.

TABLE 29
Preferred peptides for leukocyte and
fibroblast stimulation/proliferation

| SEQ ID NO: | P-number | Length | Leukocyte | Fibroblast |
|---|---|---|---|---|
| 1 | 29 | 23 | X | X |
| 2 | 2 | 23 | X | X |
| 5 | 12 | 38 | X | X |
| 6 | 13 | 23 | X | X |
| 8 | 23 | 23 | X | X |
| 10 | 25 | 23 | X | X |
| 11 | 26 | 21 | X | X |
| 12 | 27 | 19 | X | X |
| 13 | ^{27B} | 19 | X | X |
| 14 | ^{27C} | 19 | X | X |
| 15 | 30 | 23 | X | X |
| 16 | 34 | 16 | X | X |
| 17 | 35 | 17 | X | X |
| 20 | 38 | 15 | | X |
| 27 | 45 | 14 | | X |
| 28 | 46 | 15 | | X |
| 30 | 48 | 13 | | X |
| 32 | 50 | 17 | | X |
| 34 | 54 | 13 | X | |
| 45 | 66 | 13 | X | X |
| 46 | 70 | 23 | X | X |
| 50 | 74 | 13 | X | X |
| 51 | 75 | 13 | X | X |
| 55 | 80 | 23 | | X |
| 56 | 81 | 23 | | X |
| 57 | 91 | 15 | X | X |
| 58 | 92 | 13 | X | X |
| 59 | 93 | 13 | | X |
| 60 | 94 | 13 | | X |
| 61 | 95 | 13 | X | X |
| 65 | 101 | 13 | | X |
| 66 | 102 | 13 | | X |
| 71 | 107 | 19 | X | X |
| 74 | 110 | 12 | | X |
| 75 | 111 | 13 | | X |
| 77 | 113 | 13 | | X |
| 80 | 118 | 13 | | X |
| 81 | 119 | 14 | | X |
| 87 | 125 | 13 | X | X |
| 90 | 128 | 5 | X | X |
| 91 | 129 | 5 | | X |
| 92 | 130 | 5 | | X |
| 108 | 146 | 13 | X | X |
| 115 | 153 | 17 | | X |
| 116 | 154 | 13 | X | |
| 126 | 165 | 11 | | X |
| 127 | 166 | 11 | | X |
| 129 | 168 | 6 | X | X |
| 132 | 171 | 11 | | X |
| 137 | 176 | 11 | X | |
| 138 | 177 | 12 | X | |
| 139 | 178 | 11 | X | X |
| 140 | 179 | 11 | X | X |
| 141 | 180 | 11 | X | X |
| 142 | 181 | 10 | X | X |
| 143 | 182 | 10 | X | X |
| 144 | 183 | 5 | X | X |
| 145 | 184 | 5 | X | X |
| 159 | 508 | 23 | X | X |
| 162 | 67 | 23 | X | X |
| 164 | 69 | 18 | | X |
| 165 | 97 | 13 | X | X |

### Example 11

### Synergistic Effects With Lysozyme

Synergy between lytic peptides and lysozyme was assayed. Sterilized milk was inoculated with bacteria to 5×10⁵ per ml. Peptide Shiva-10 (SEQ ID NO:4) was added to 10 μg/ml, and chicken lysozyme was added to 1 mg/ml. The percent killing of bacteria was determined.

TABLE 30

| | Staph. aureus | Pseud. aeruginosa |
|---|---|---|
| Peptide and lysozyme | 0% | 100% |
| Peptide | 0% | 0% |
| Lysozyme | 0% | 0% |

Synergy between cecropin SB-37 (SEQ ID NO:5) and lysozyme was determined against Pseudomonas syringae pv. tabaci (PSPT), Pseudomonas solanacearum (PS), Erwinia caratovora subsp. carotova (EC), and Xanthomonas campestris pv. campestris (XC). LD₅₀ (μM) values were determined.

TABLE 31

| | | | SB-37 and |
|---|---|---|---|
| | SB-37 | Lysozyme | Lysozyme |
| PSPT | 5.20 | > | 0.19 |
| PS | 64.0 | > | 16.0 |
| EC | 1.48 | > | 0.44 |
| XC | 0.57 | > | 0.027 |

> indicates greater than 1000.

Synergy between Shiva- 1 and lysozyme was determined. The percent viability of Pseudomonas aeruginosa was determined relative to blank controls. Lysozyme was used at the same molar concentration as the peptide.

TABLE 32

| Peptide | | | | Shiva-1 and |
|---|---|---|---|---|
| concentration | | | Lysozyme | Lysozyme |
| (μM) | SB-37 | Shiva-1 | (1×) | (1×) |
| 0 | 100 | 100 | 100 | 100 |
| 0.01 | 100 | 100 | 100 | 56.6 |
| 0.1 | 79.4 | 69.6 | 82.2 | 25.8 |
| 1 | 48.8 | 37.9 | 52.1 | 4.4 |
| 5 | 38.5 | 1.5 | 7.9 | 0.2 |
| 7.5 | 0.7 | 0.1 | 0.6 | 0 |
| 25 | 0 | 0 | 0.4 | 0 |

Synergy between Shiva-1 and lysozyme was determined. The percent viability of gram positive S. intermedius 19930, S. intermedius 20034, and S. aureus was determined relative to blank controls. Lysozyme was used at ten times the molar concentration as the peptide.

TABLE 33
S. intermedius 19930

| Peptide | | | | Shiva-1 and |
|---|---|---|---|---|
| concentration | | | Lysozyme | Lysozyme |
| (μM) | SB-37 | Shiva-1 | (10×) | (10×) |
| 0 | 100 | 100 | 100 | 100 |
| 0.01 | 100 | 100 | 100 | 100 |
| 0.1 | 94.7 | 81.8 | 100 | 79.2 |
| 0.5 | 69.4 | 65.0 | 81.3 | 65.1 |
| 1 | 42.5 | 42.1 | 53 | 43 |
| 5 | 36.1 | 35.2 | 49.5 | 17.2 |
| 10 | 5.6 | 1.2 | 34.4 | 1.1 |
| 50 | 0 | 0 | 22 | 0 |

TABLE 34
S. intermedius 20034

| Peptide | | | | Shiva-1 and |
|---|---|---|---|---|
| concentration | | | Lysozyme | Lysozyme |
| (μM) | SB-37 | Shiva-1 | (10×) | (10×) |
| 0 | 100 | 100 | 100 | 100 |
| 0.01 | 100 | 100 | 100 | 100 |
| 0.25 | 85.4 | 87.1 | 100 | 85.1 |
| 0.5 | 68.0 | 80.0 | 59.0 | 53.4 |
| 0.75 | 62.2 | 60.1 | 42.3 | 41.0 |
| 5 | 35.1 | 4.1 | 38.3 | 4.3 |
| 50 | 0 | 0 | 10.0 | 0 |

TABLE 35
S. aureus

| Peptide | | | | Shiva-1 and |
|---|---|---|---|---|
| concentration | | | Lysozyme | Lysozyme |
| (μM) | SB-37 | Shiva-1 | (10×) | (10×) |
| 0 | 100 | 100 | 100 | 100 |
| 0.01 | 100 | 100 | 100 | 100 |
| 0.1 | 100 | 100 | 100 | 100 |
| 0.5 | 81.0 | 50.1 | 100 | 100 |
| 1 | 47.5 | 24.4 | 51.0 | 31.2 |
| 5 | 31.8 | 15.9 | 18.4 | 8.2 |
| 10 | 5.6 | 4.5 | 13.3 | 4.5 |
| 50 | 1.9 | 1.6 | 9.5 | 1.4 |

Synergy experiments can also be performed using peptides in the presence of EDTA, which potentiates the peptides additively or synergistically.

### Example 12

### Synergistic Effects With Antibiotics

Synergy between peptide Shiva-10 (SEQ ID NO:4) and various antimicrobial agents was investigated against Escherichia coli 25922. The following table illustrates the beneficial effects of combining the peptide with the agents, where the numbers are the minimum bactericidal concentration (MBC; μg/mL).

TABLE 36

| Agent | Without peptide | With peptide | |
|---|---|---|---|
| Shiva-10 | 50 | n/a | |
| Ticarcillin | 100 | 50 | (15 μg/mL peptide) |
| Cefoperazone | 150 | 2.5 | (15 μg/mL peptide) |
| Doxycycline | 5 | 1 | (15 μg/mL peptide) |
| Neomycin | 100 | 5 | (5 μg/mL peptide) |
| Amikacin | 150 | 50 | (5 μg/mL peptide) |
| Tetracycline | 10 | 2.5 | (5 μg/mL peptide) |

Synergy between peptide Shiva-10 (SEQ ID NO:4) and various antimicrobial agents was investigated against Staph. aureus 29213. The following table illustrates the beneficial effects of combining the peptide with the agents, where the numbers are the minimum bactericidal concentration (MBC; μg/mL).

TABLE 37

| Agent | Without peptide | With 5 μg/mL peptide |
|---|---|---|
| Shiva-10 | 200 | n/a |
| Ampicillin | 5 | 2.5 |
| Ticarcillin | 25 | 15 |
| Cefoperazone | 10 | 2.5 |
| Tobramycin | 25 | 10 |
| Tetracycline | 10 | 1 |

Synergy between peptide FLAK 26AM (P35; SEQ ID NO:17) and various antimicrobial agents was investigated against Staph. aureus 29213 MBC. The following table illustrates the beneficial effects of combining the peptide with the agents, where the numbers are the minimum bactericidal concentration (MBC; μg/mL). This experiment determined the peptide MBC in the absence of the antimicrobial agent, or in the presence of the indicated concentration of antimicrobial agent

TABLE 38

| Agent | MBC of peptide |
|---|---|
| FLAK 26AM alone | 50 |
| Vancomycin (1 ppm) | 32 |
| Cefoperazone (0.25 ppm) | 20 |

Synergy between doxacycline and various peptides was investigated against P. aeruginosa 27853. The following table illustrates the beneficial effects of combining doxacycline and the peptides, where the numbers are the minimum bactericidal concentration (MBC; μg/mL). When combined with the peptides, the doxacycline was held at 10 ppm concentration.

TABLE 39

| | Without | With |
|---|---|---|
| Agent | doxacycline | doxacycline |
| Doxacycline | n/a | 100 |
| SB-37 (P5; SEQ ID NO: 3) | 200 | 30 |
| FLAK 26AM (P35; SEQ ID NO: 17) | 50 | 32 |

Synergy between tetracycline and various peptides was investigated against Escherichia coli 25922 MBC. The following table illustrates the beneficial effects of combining tetracycline and the peptides, where the numbers are the minimum bactericidal concentration (MBC; μg/mL). When combined with the peptides, the concentration of tetracycline was held at 1.5 ppm.

TABLE 40

| | Without | With |
|---|---|---|
| Agent | tetracycline | tetracycline |
| Tetracycline | n/a | 10 |
| FLAK 06AM (P27; SEQ ID NO: 12) | 75 | 25 |
| FLAK 26AM (P35; SEQ ID NO: 17) | 50 | 20 |

### Example 13

### Synergistic Effects With Chemotherapy Agents

Other investigators have reported that lytic peptides which are inhibitory to cancer cells will act synergistically with conventional cancer chemotherapy drugs. The FLAK peptides are no exception. Table 41 below demonstrates for example that selected FLAK peptides are synergistic with Tamoxifen in the inhibition of the MCF7 line of breast cancer cells. Table 42 lists other more active anti-cancer peptide candidates for synergistic application with Tamoxifen or other cancer therapy drugs.

Tables 41 and 42 also show toxicity of the selected peptides against RBCs, WBCs, and WI38 cells. When used at very low non-toxic levels selected anti-cancer peptides can synergistically potentiate other chemotherapy agents to permit their effective use at substantially lower dose levels with consequently fewer side effects.

TABLE 41
Synergy of FLAK peptides with tamoxifen on MCF7 cells

| | | Active agent | LD50 on MCF7 cells | | |
|---|---|---|---|---|---|
| SEQ ID NO: | | MCF7 | Peptide | Tamox. | Total conc. |
| (P No.) | Agent | LD50 μg/ml | conc. μg/ml | conc. μg/ml | μg/ml |
| | Tamoxifen | 20 | 0 | 20 | 20 |
| 164 (69) | Alone | 79 | 2.5 | 4.6 | 7.1 |
| | With Tamox. | | | | |
| 145 (184) | Alone | 240 | 10 | 4 | 14 |
| | With Tamox. | | | | |
| 121 (160) | Alone | 240 | 11 | 3.7 | 14.7 |
| | With Tamox. | | | | |
| 106 (144) | Alone | 310 | 35 | 7.7 | 42.7 |
| | With Tamox. | | | | |

| SEQ ID NO: | MCF7 LD50 | RBC LD50 | WI38 LD50 | WBC LD50 |
|---|---|---|---|---|
| (P No.) | μg/ml | μg/ml | μg/ml | μg/ml |
| 164 (69) | 79 | 900 | 60 | 140 |
| 145 (184) | 240 | 850 | 1000 | 410 |
| 121 (160) | 240 | >1000 | 700 | 900 |
| 106 (144) | 310 | 600 | 740 | 320 |
| 17 (35) | 9 | 200 | 25 | 25 |
| 32 (50) | 32 | 420 | 40 | 420 |
| 20 (38) | 17 | 350 | 100 | 54 |

TABLE 42
Other highly active peptide candidates for
synergistic anti-cancer applications

| SEQ ID NO: | MCF7 LD50 | RBC LD50 | WI38 LD50 | WBC LD50 |
|---|---|---|---|---|
| (P No.) | μg/ml | μg/ml | μg/ml | μg/ml |
| 17 (35) | 9 | 200 | 25 | 25 |
| 32 (50) | 32 | 420 | 40 | 420 |
| 20 (38) | 17 | 350 | 100 | 54 |

### Example 14

### Synergistic Effects With Growth Factors

It has been shown above in Example 17 and Table 23 that certain of the FLAK peptides are synergistic with other mitogens or growth factors in the stimulatory and/or proliferative properties of the peptides.

### Example 15

### Synergistic Effects With Nalidixic Acid and Chloramphenicol

The synergistic effects of the inventive peptides with either chloramphenicol or nalidixic acid against efflux mutants of Pseudomonas aeruginosa were investigated. The MIC values were determined for either nalidixic acid or chloramphenicol alone as baselines. Peptides were added at their ¼ MIC concentration, and the concentration of either nalidixic acid or chloramphenicol to arrive at the MIC was determined. Table 43 shows the peptides' synergistic effects with nalidixic acid against P. aeruginosa H374, Table 44 shows the peptides' synergistic effects with nalidixic acid against P. aeruginosa H774, and Table 45 shows the peptides' synergistic effects with chloramphenicol against P. aeruginosa H374. The fractional inhibitory concentration (FIC) index was used to determine synergy between peptides and antibiotics. Two-fold serial dilutions of antibiotic were tested in the presence of a constant amount of peptide, equal to one quarter of peptide MIC. The FIC index was determined as follows: FIC=0.25+MIC_{antibiotic} in combination/MIC_{antibiotic} alone. An FIC index of 0.5 or less is considered as synergy.

TABLE 43

| | |
|---|---|
| Peptide in | P. aeruginosa H374 |

| Combination | MIC _{Nal-comb.} | |
|---|---|---|
| (¼ MIC) | (μg/ml) | FIC*_{Index} |
| Nal alone | 5000 | — |
| P12 | 2500 | 0.75 |
| P23 | 2500 | 0.75 |
| P24 | 5000 | 1.25 |
| P25 | 2500 | 0.75 |
| P26 | 2500 | 0.75 |
| P27 | 2500 | 0.25 |
| P30 | 5000 | 1.25 |
| P31 | 2500 | 0.75 |
| P34 | 2500 | 0.75 |
| P35 | 10,000 | 2.25 |
| P37 | 2500 | 0.75 |
| P39 | 1250 | 0.5 |
| P41 | 5000 | 1.25 |
| P42 | 5000 | 1.25 |
| P43 | 5000 | 1.25 |
| P44 | 5000 | 1.25 |
| P45 | 2500 | 0.75 |
| P46 | 2500 | 0.75 |
| P49 | 2500 | 0.75 |
| P50 | 5000 | 1.25 |
| P54 | 5000 | 1.25 |
| P55 | 5000 | 1.25 |
| P56 | 2500 | 0.75 |
| P59 | 2500 | 0.75 |
| P60 | 1250 | 0.5 |
| P61 | 5000 | 1.25 |
| P64 | 5000 | 1.25 |
| P66 | 5000 | 1.25 |
| P69 | 2500 | 0.75 |
| P71 | 2500 | 0.75 |
| P72 | 2500 | 0.75 |
| P73 | 2500 | 0.75 |
| P75 | 2500 | 0.75 |

| | |
|---|---|
| Peptide in | P. aeruginosa H374 |

| Combination | MIC _{Nal-comb.} | |
|---|---|---|
| (¼ MIC) | (μg/ml) | FIC_{Index} |
| P80 | 2500 | 0.75 |
| P81 | 5000 | 1.25 |
| P97 | 5000 | 1.25 |
| P100 | 2500 | 0.75 |
| P101 | 5000 | 1.25 |
| P102 | 5000 | 1.25 |
| P103 | 625 | 0.375 |
| P109 | 2500 | 0.75 |
| P110 | 2500 | 0.75 |
| P111 | 2500 | 0.75 |
| P118 | 2500 | 0.75 |
| P119 | 2500 | 0.75 |
| P124 | 2500 | 0.75 |
| P146 | 625 | 0.375 |
| P150 | 1250 | 0.5 |
| P153 | 5000 | 1.25 |
| P157 | 2500 | 0.75 |
| P177 | 5000 | 1.25 |
| P300 | 312 | 0.312 |
| P301 | 625 | 0.375 |
| P306 | 5000 | 1.25 |
| P307 | 625 | 0.375 |
| P504 | 5000 | 1.25 |
| P508 | 5000 | 1.25 |
| P510 | 625 | 0.375 |

TABLE 44

P. aeruginosa H744

| Peptide in | MIC _{Nal-comb.} | |
|---|---|---|
| combination | (μg/ml) | FIC*_{Index} |
| Nal alone | 624 | — |
| P12 | 312 | 0.75 |
| P23 | 624 | 1.25 |
| P24 | 624 | 1.25 |
| P25 | 156 | 0.5 |
| P26 | 624 | 1.25 |
| P27 | 624 | 1.25 |
| P30 | 624 | 1.25 |
| P31 | 624 | 1.25 |
| P34 | 624 | 1.25 |
| P35 | 624 | 1.25 |
| P37 | 624 | 1.25 |
| P39 | 624 | 1.25 |
| P41 | 624 | 1.25 |
| P42 | 624 | 1.25 |
| P43 | 624 | 1.25 |
| P44 | 624 | 1.25 |
| P45 | 624 | 1.25 |
| P46 | 624 | 1.25 |
| P49 | 624 | 1.25 |
| P50 | 624 | 1.25 |
| P54 | 624 | 1.25 |
| P55 | 624 | 1.25 |
| P56 | 624 | 1.25 |
| P59 | 624 | 1.25 |
| P60 | 624 | 1.25 |
| P61 | 624 | 1.25 |
| P64 | 624 | 1.25 |
| P66 | 624 | 1.25 |
| P69 | 312 | 0.75 |
| P71 | 624 | 1.25 |
| P72 | 312 | 0.75 |
| P73 | 624 | 1.25 |
| P75 | 624 | 1.25 |

P. aeruginosa H744

| Peptide in | MIC _{Nal-comb.} | |
|---|---|---|
| combination | (μg/ml) | FIC_{Index} |
| P80 | 624 | 1.25 |
| P81 | 624 | 1.25 |
| P97 | 78 | 0.375 |
| P100 | 624 | 1.25 |
| P101 | 624 | 1.25 |
| P102 | 624 | 1.25 |
| P103 | 624 | 1.25 |
| P109 | 624 | 1.25 |
| P110 | 624 | 1.25 |
| P111 | 624 | 1.25 |
| P118 | 624 | 1.25 |
| P119 | 624 | 1.25 |
| P124 | 624 | 1.25 |
| P146 | 624 | 1.25 |
| P150 | 312 | 0.75 |
| P153 | 624 | 1.25 |
| P157 | 624 | 1.25 |
| P177 | 312 | 0.75 |
| P300 | 156 | 0.5 |
| P301 | 624 | 1.25 |
| P306 | 312 | 0.75 |
| P307 | 156 | 0.5 |
| P504 | 1248 | 2.25 |
| P510 | 624 | 1.25 |

TABLE 45

| | |
|---|---|
| Peptide in | P. aeruginosa H374 |

| Combination | MIC _{Cm-comb.} | |
|---|---|---|
| (¼ MIC) | (μg/ml) | FIC*_{Index} |
| Cm alone | 16 | — |
| P12 | 16 | 1.25 |
| P23 | 8 | 0.75 |
| P24 | 16 | 1.25 |
| P25 | 4 | 0.5 |
| P26 | 8 | 0.75 |
| P27 | 8 | 0.75 |
| P30 | 16 | 1.25 |
| P31 | 16 | 1.25 |
| P34 | 16 | 1.25 |
| P35 | 16 | 1.25 |
| P37 | 4 | 0.5 |
| P39 | 8 | 0.75 |
| P41 | 16 | 1.25 |
| P42 | 16 | 1.25 |
| P43 | 16 | 1.25 |
| P44 | 16 | 1.25 |
| P45 | 16 | 1.25 |
| P46 | 8 | 0.75 |
| P49 | 8 | 0.75 |
| P50 | 16 | 1.25 |
| P54 | 16 | 1.25 |
| P55 | 16 | 1.25 |
| P56 | 16 | 1.25 |
| P59 | 8 | 0.75 |
| P60 | 4 | 0.5 |
| P61 | 16 | 1.25 |
| P64 | 16 | 1.25 |
| P66 | 16 | 1.25 |
| P69 | 8 | 0.75 |
| P71 | 8 | 0.75 |
| P72 | 8 | 0.75 |
| P73 | 8 | 0.75 |
| P75 | 8 | 0.75 |

| | |
|---|---|
| Peptide in | P. aeruginosa H374 |

| Combination | MIC _{Cm-comb.} | |
|---|---|---|
| (¼ MIC) | (μg/ml) | FIC_{Index} |
| P80 | 4 | 0.5 |
| P81 | 16 | 1.25 |
| P97 | 16 | 1.25 |
| P100 | 16 | 1.25 |
| P101 | 16 | 1.25 |
| P102 | 16 | 1.25 |
| P103 | 8 | 0.75 |
| P109 | 16 | 1.25 |
| P110 | 16 | 1.25 |
| P111 | 16 | 1.25 |
| P113 | 16 | 1.25 |
| P118 | 16 | 1.25 |
| P119 | 16 | 1.25 |
| P124 | 16 | 1.25 |
| P146 | 4 | 0.5 |
| P150 | 8 | 0.75 |
| P153 | 8 | 0.75 |
| P157 | 8 | 0.75 |
| P177 | 8 | 0.75 |
| P300 | 16 | 1.25 |
| P301 | 16 | 1.25 |
| P306 | 8 | 0.75 |
| P307 | 2 | 0.375 |
| P504 | 16 | 1.25 |
| P508 | 8 | 0.75 |
| P510 | 4 | 0.5 |

### Example 16

### Activity Against Drug Resistant Strains

Peptides were assayed for their activity against tobramycin sensitive and resistant strains. As shown in the following Table 46, peptides P56 (SEQ ID NO:36), P74 (SEQ ID NO:50), and P125 (SEQ ID NO:87) showed greater activity against tobramycin resistant (tr) Pseudomonas ATCC 13096 than against tobramycin sensitive (ts) Pseudomonas ATCC 27853. The same three peptides showed greater activity against clinical tobramycin resistant strain 960890198-3c (Table 46).

TABLE 46

| Peptide | tr Pseudomonas 13096 | ts Pseudomonas 27853 |
|---|---|---|
| SEQ ID NO: 36 (P56) | 16 | 125 |
| SEQ ID NO: 50 (P74) | 16 | 125 |
| SEQ ID NO: 87 (P125) | 4 | 31 |

TABLE 47

| | tr Pseudomonas | ts Pseudomonas |
|---|---|---|
| Peptide | 960890198-3c | 27853 |
| SEQ ID NO: 36 (P56) | >50 | 125 |
| SEQ ID NO: 50 (P74) | 25 | 125 |
| SEQ ID NO: 87 (P92) | 50 | 63 |

### Example 17

### Wound Healing

The inventive peptides can be used in compositions for topical or systemic delivery in wound healing applications. The compositions can be a liquid, cream, paste, or other pharmaceutically acceptable formulation. The compositions may contain other biologically active agents. The compositions may contain pharmaceutically acceptable carriers.

FLAK peptides have demonstrated high potency against the bacteria most associated with wound infections, S. aureus, S. pyogenes and P. aeruginosa (e.g. Tables 5, 6, and 7). The peptides have also demonstrated the ability to aid in the healing of wounds and perhaps reduce inflammation. These properties are all essential attributes of wound and wound infection treatment products.

Those peptides presently preferred for wound healing, shown in Table 48 below, are peptides that were preferred for either, or both, leukocyte or fibroblast stimulation and for anti-bacterial properties.

TABLE 48
Presently preferred peptides for wound healing

| SEQ ID NO: | P No. |
|---|---|
| 1 | 1 |
| 2 | 2 |
| 5 | 12 |
| 6 | 13 |
| 8 | 23 |
| 10 | 25 |
| 11 | 26 |
| 12 | 27 |
| 13 | 27B |
| 14 | 27C |
| 15 | 30 |
| 16 | 34 |
| 17 | 35 |
| 20 | 38 |
| 27 | 45 |
| 28 | 46 |
| 30 | 48 |
| 32 | 50 |
| 34 | 54 |
| 45 | 66 |
| 46 | 70 |
| 50 | 74 |
| 51 | 75 |
| 55 | 80 |
| 56 | 81 |
| 57 | 91 |
| 58 | 92 |
| 59 | 93 |
| 60 | 94 |
| 61 | 95 |
| 65 | 101 |
| 66 | 102 |
| 71 | 107 |
| 74 | 110 |
| 75 | 111 |
| 77 | 113 |
| 80 | 118 |
| 81 | 119 |
| 87 | 125 |
| 90 | 128 |
| 91 | 129 |
| 92 | 130 |
| 93 | 131 |
| 108 | 146 |
| 115 | 153 |
| 116 | 154 |
| 126 | 165 |
| 127 | 166 |
| 129 | 168 |
| 132 | 171 |
| 137 | 176 |
| 138 | 177 |
| 139 | 178 |
| 140 | 179 |
| 141 | 180 |
| 142 | 181 |
| 143 | 182 |
| 144 | 183 |
| 145 | 184 |
| 159 | 508 |
| 162 | 67 |
| 164 | 69 |
| 165 | 97 |

### Example 8

### Wound Healing With FLAK Peptides Demonstrated In-vivo

U.S. Pat. No. 5,861,478 disclosed in vivo wound healing in a rat model in which the healing agent was the peptide LSB-37. LSB-37 is identified herein as SEQ. NO. 150 (peptide P306), and is evaluated herein by way of comparision with the smaller FLAK peptides which are the subject of the present invention. As set forth in Example 17 the FLAK peptides, based on extensive in vitro assays, offer promise as wound healing agents. This has been demonstrated in in vivo testing of selected FLAK (and other) peptides in a small animal topical wound healing model developed for this purpose.

The objective of the study was to evaluate the effects of certain selected peptides on (i) the rate of wound closure, (ii) inflammatory response, and (iii) epidermal thickening on a chemically induced skin burn wound. The hairless rat was chosen as a suitable test model. Female hairless rats of 100 to 150 grams weight and 8 to 12 weeks age were used in the study.

Phenol based skin peels reported in the literature and in private communications were found to be systemically toxic for use in this study, where six separate test patches (peels) with a total surface area of >2 square inches were induced on a single animal. As an alternative, 70% trichloroacetic (TCA) dissolved in 70% ethanol was employed to induce the dermal erosion patches. With 30 minute peel occlusions resulted in third degree burns with complete erosion of the epidermis and dermis. As the chemical burn agent, the TCA treatment inflicted on the rats far less trauma and mortality than occurred with the Phenol model.

The experimental Protocol procedure steps were as follows:
1. The animal was anesthetized (40 mg/kg Phenobarbital).
2. Color photographs of the animal's back (with six separate peels) were taken before each treatment and daily thereafter.
3. Rat skin surface was prepared by wiping with 70% ethanol. Filter paper discs (1.1 cm diameter) were soaked in 70% TCA/ethanol.
4. The discs were placed on the back of the hairless rat for 30 minutes [6 disks providing for 2 control (no peptide treatment) disks and 4 disks for peels to receive peptide treatment.]
5. After a 30 minute burn the discs were removed. Twenty four hours later, different peptide solutions (1500 ppm in saline) were applied to four peels, and saline was applied to the two control peels.
6. Peptide solutions (and saline for the controls) were applied to the six wounds with a soft brush each day thereafter.
7. It took approximately one month for the wounds to heal (complete skin closure with stabilized epidermis), after which the animal was sacrificed.
8. The treated skin was harvested, section stained with trichrome, and mounted on slides.

The percentage of wound closure for each peel (six sites) was measured each day until the animal was sacrificed. The percentage closure was determined by measuring on the animal photographs the area of the remaining scab relative to the area of the initial scar after the burn. These measurements were made by digitizing and analyzing the peels using the Sigma Plot ProScan 4 program.

After full wound closure, a portion of each peel still had a red, inflamed area which was quantitated by the Sigma Plot analysis of the animal photgraph, as a percentage of the total healed scar. This provided a measure of the post-TCA burn treatment of the inflammatory response in each peel site.

The extent of epidermal thickening (hyperkeratosis) at each site was also determined by measurement with the Sigma Plot program applied to the stained section slides of the various wound areas and the normal untreated skin (control) surrounding the peels. At magnifications of 100× to 320×, the microphotographs of the color slides provided a powerful tool for such quantification of the extent of hyperkeratosis evident in each peel.

Treatment of the section slides with selective stains produced identifiable evidence of the presence of both leukocyte and fibroblast cells in the wound areas. This was also quantified by the Sigma Plot program. It proved to be a useful tool in determining, in vivo, the mechanisms by which different peptides affected the wound healing process, including leukocyte stimulation/proliferation and fibroblast stimulation/proliferation and chemotactic effects of the peptides in wound healing in-vivo.

The above described animal model and protocols were employed in the testing of approximately 20 of the peptides listed in Table 48 (and other peptides for comparison) as preferred FLAK peptides for wound healing. By way of example, the following results on an experiment with four peptides evaluated in a single animal are shown in Table 49. These peptides are SEQ ID NO:66 (P102), SEQ ID NO:71 (P107), SEQ ID NO:115 (P153), and SEQ ID NO:119 (P157). Peptide SEQ ID NO:71 (P107) is not a FLAK peptide, but is a derivative of LSB-37 (SEQ ID NO:150; P06). In earlier experiments these two peptides have been shown to have very similar wound healing properties in vivo. SEQ ID NO:119 (P157) is a non-FLAK peptide, reported in the literature, which is a comparison peptide.

Table 49 supports the conclusion that several peptides evaluated for post wound treatments demonstrated the ability to limit post-TCA burn inflammatory responses. SEQ ID NO:71 and SEQ ID NO:115 were superior in this respect and also showed the lowest evidence of hyperkeratosis (epidermal thickening). Since the experiment was carried to full wound closure at 26 days, these same two peptides displayed a small advantage in rate of wound closure over the other peptides and no peptide in post wound treatment. These two peptides also showed substantially no hyperkeratosis as compared to the TCA burn untreated control.

Overall the best wound healing activity was displayed by the two above cited peptides. However, the experiment was conducted under sterile conditions that do not usually occur in real life animal wound situations. Because such topical wounds are subject to infection, it must be considered that the superior anti-bacterial properties of both SEQ ID NO:66 (P102) and SEQ ID NO:1 15 (P153) make them logical candidates for wound healing applications.

TABLE 49
Selected in-vivo FLAK peptide wound healing example (Rat model)

| | | | | Leukocyte | Fibroblast |
|---|---|---|---|---|---|
| | Wound | Inflammatory | Epidermal | cells in test | cells in test |
| | closure | response area | thickening | area | area |
| | % of initial | % of healed | % of control | % of normal | % of normal |
| | wound | scar | (TCA only) | skin | skin |
| SKIN SAMPLE | | | | | |
| Normal skin | N/A | N/A | N/A | 100 | 100 |
| TCA burn untreated | 98.4 | 15 | 30 | 200 | 275 |
| (control) | | | | | |
| Burns treated by peptide: | | | | | |
| SEQ ID NO: 66 (P102) | 96.7 | 27 | 50 | 370 | 220 |
| SEQ ID NO: 71 (P107) | 100 | 0 | 33 | 400 | 420 |
| SEQ ID NO: 115 (P153) | 99.1 | 7 | 25 | 235 | 350 |
| SEQ ID NO: 119 (P157) | 95.2 | 25 | 80 | 265 | 450 |

### Example 19

### Treatment of Cystic Fibrosis (CF)

CF is the most common autosomal recessive genetic disorder in North America, causing inflammation and infection in the lungs of 30,000 children a year in the USA. Over 90% of CF lung infections are caused by P. aeruginosa and over 95% of these patients die from lung damage. Certain FLAK peptides are active against multi-drug resistant strains Pseudomonas aeruginosa and against clinical isolates from CF patients (Tables 9, 43 and 44). These include strains resistant to TOBI, the current drug of choice for this condition. In addition, peptides such as these (alpha-helical peptides) have previously been shown to have anti-inflammatory properties (Scott et al., J. Immunol. 165: 3358-3365, 2000) and it would therefore not be surprising if FLAK peptides also exhibited this property. The combination of an anti-inflammatory and an anti-infective role makes these peptides extremely good candidates as novel therapeutics for the CF lung.

### Example 20

### Treatment of Sexually Transmitted Diseases (STDs)

Sexually transmitted diseases (STD) are a significant problem in North America costing the US alone $10 billion a year in treatment costs. One of the key problems is the increasing incidence of anti-fungal, primarily fluconazole, resistant strains of Candida including species such as C. albicans, C. glabrata and C. tropicalis. Certain FLAK peptides have demonstrated significant activity against all three of these species (Tables 13 and 10) and present a very viable opportunity for the development of a topical anti-fungal agent to prevent the spread of fungal disease. There is evidence in the literature suggesting that FLAK peptides may also have activity against other STD agents including viruses and bacteria which suggests that a broad spectrum application may also be possible. Certain FLAK peptides demonstrate a broad spectrum of activity (Tables 12 and 13).

### Example 21

### Treatment of Acne

Acne is caused by a combination of infection and inflammation that leads to tissue damage and scarring. FLAK peptides have demonstrated activity against the primary bacteria isolated from acne sores, Propionibacterium acne and also will likely exhibit anti-inflammatory activities (Scott et al., J. Immunol. 165: 3358-3365, 2000). In addition, the FLAK peptides have also shown a propensity to increase the speed and efficiency of wound healing, increase the proliferation of fibroblasts and increase collagen and laminin production. All of these attributes provide compelling evidence for the application of FLAK peptides to the treatment of acne either as a clinical therapeutic or as a cosmeceutical.

### Example 22

### Cosmetics Applications

The attributes of FLAK peptides such as collagen stimulation, fibroblast stimulation and wound healing make the potential for the use of such peptides in cosmetics such as anti-aging and rejuvination products very appealing.

### Example 23

### Use of FLAK Peptides in the Food Industry

The primary causes of diseases related to the food industry are Gram-negative bacteria such as Salmonella typhimurium and Escherichia coli. A number of FLAK peptides demonstrated specific activity against these organisms (Tables 7 and 12). The application of such peptides to the treatment and also prevention of food borne disease is therefore an appealing application. For example the use of such peptides for the decontamination of food preparation surfaces is a specific and potentially novel application.

### Example 24

### Systemic Application of Peptides in Serum

A series of peptides were introduced into sheep serum at 1280 ug/ml and incubated at 37° C. for either 30 minutes or 2 hours (Table 50). Subsequently, the serum MICs against Pseudomonas aeruginosa were conducted to determine extent of serum inactivation of the peptides. Of the peptides tested, two (P153 and P508) were soluble at 1280 μg/ml in 70% serum and their activities were only modestly decreased by exposure to serum. This suggests that P153 and P508 are able to function in serum and are good candidates for a systemic application.

TABLE 50
Serum inactivation of peptides

| | | MIC 30 min treatment | | MIC 2 hr treatment | |
|---|---|---|---|---|---|
| Peptide | Solubility | (μg/ml) | | (μg/ml) | |
| P24 | Precipitated | 40 | 20 | 20 | 20 |
| P31 | Precipitated | 20 | 20 | 20 | 20 |
| P69 | Precipitated | 20 | 20 | 20 | 20 |
| P81 | Precipitated | 20 | 20 | 20 | 20 |
| P153 | Soluble | 10 | 5 | 20 | 5 |
| P508 | Soluble | 40 | 20 | 40 | 20 |
| KB142 | Precipitated | 20 | 20 | 20 | 20 |
| KB146 | Precipitated | 20 | 20 | 20 | 20 |

### Example 25

### Collagen and Laminin Stimulation by FLAK Peptides

Fibroblast cell lines were cultured under standard conditions and assayed for collagen and laminin using an ELISA system manufactured by Panvera (Madison, Wis.). Antibodies for collagen and laminin manufactured by Takara Shuzo Co., Ltd Japan. Table 51 below shows that one of the four peptides displayed significant stimulation of collagen and laminin production. The other three peptides tested neither stimulated nor inhibited production (i.e. no effect was observed).

TABLE 51
Collagen and laminin stimulation

| Peptide | Collagen stimulation | Laminin stimulation |
|---|---|---|
| TGFβ (control) | 60% | — |
| P153 (SEQ ID NO: 115) | 120% | 32% |
| P165 (SEQ ID NO: 126) | 0% | 0% |
| P94 (SEQ ID NO: 60) | 0% | 0% |
| P12 (SEQ ID NO: 5) | 0% | 0% |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention.

1. An isolated peptide comprising at least three different amino acid residues selected from the group consisting of phenylalanine, leucine, alanine, and lysine, wherein:
the peptide is from 10 to 22 amino acid residues in length;
the first amino acid residue in the peptide is valine; and
at least 80% of the peptide's amino acid residues are selected from the group consisting of phenylalanine, leucine, alanine, and lysine.

2. The peptide of claim 1 wherein the peptide is SEQ ID NO:15, SEQ ID NO:30, SEQ ID NO:32, SEQ ID NO:116, SEQ ID NO:141, SEQ ID NO:152, SEQ ID NO:155, SEQ ID NO:156, or SEQ ID NO:157:

3. The peptide of claim 1 wherein after its first amino acid residue the peptide has only leucine, alanine, and lysine amino acid residues.

4. The peptide of claim 3 wherein the peptide is SEQ ID NO:15, SEQ ID NO:30, SEQ ID NO:32, SEQ ID NO:141, SEQ ID NO:152, SEQ ID NO:155, SEQ ID NO:156, or SEQ ID NO:157.

5. The peptide of claim 3 that is SEQ ID NO:32.

6. A composition comprising at least one peptide according to claim 1.

7. The composition of claim 6 wherein the peptide is SEQ ID NO:15, SEQ ID NO:30, SEQ ID NO:32, SEQ ID NO:116, SEQ ID NO:141, SEQ ID NO:152, SEQ ID NO:155, SEQ ID NO:156, or SEQ ID NO:157.

8. The composition of claim 6 wherein after its first amino acid residue the peptide has only leucine, alanine, and lysine amino acid residues.

9. The composition of claim 9 wherein the peptide is SEQ ID NO:15, SEQ ID NO:30, SEQ ID NO:32, SEQ ID NO:141, SEQ ID NO:152, SEQ ID NO:155, SEQ ID NO:156, or SEQ ID NO:157.

10. The composition of claim 8 comprising the peptide of SEQ ID NO:32

11. The composition of claim 6 that is antimicrobial.

12. The composition of claim 6 that is antibacterial and/or antifungal.

13. The composition of claim 6 that is effective for inhibiting at least one microorganism selected from the group consisting of: Acinetobacter baumannii, Candida albicans, Candida glabrata, Candida guilliermondii, Candida tropicalis, Escherichia coli, Propionibacterium acnes, Pseudomonas aeruginosa, Salmonella typhimurium, Staphylococcus aureus, Staphylococcus epidermidis, Staphylococcus intermedius, Streptococcus pneumoniae, Streptococcus pyogenes.

14. A method of treating the skin or wound of an animal comprising: contacting the skin or wound with a composition comprising at least one peptide according to claim 1.

15. The method of claim 14 wherein the peptide is SEQ ID NO:15, SEQ ID NO:30, SEQ ID NO:32, SEQ ID NO:116, SEQ ID NO:141, SEQ ID NO:152, SEQ ID NO:155, SEQ ID NO:156, or SEQ ID NO:157.

16. The method of claim 14 wherein after its first amino acid residue the peptide has only leucine, alanine, and lysine amino acid residues.

17. The method of claim 16 wherein the peptide is SEQ ID NO:15, SEQ ID NO:30, SEQ ID NO:32, SEQ ID NO:141, SEQ ID NO:152, SEQ ID NO:155, SEQ ID
NO:156, or SEQ ID NO:157.

18. The method of claim 16 wherein the peptide is SEQ ID NO:32.

19. The method of claim 14 wherein the composition is antimicrobial.

20. The method of claim 14 wherein the composition is antibacterial and/or antifungal.

21. The method of claim 14 wherein the composition is effective for inhibiting at least one microorganism selected from the group consisting of: Acinetobacter baumannii, Candida albicans, Candida glabrata, Candida guilliermondii, Candida tropicalis, Escherichia coli, Propionibacterium acnes, Pseudomonas aeruginosa, Salmonella typhimurium, Staphylococcus aureus, Staphylococcus epidermidis, Staphylococcus intermedius, Streptococcus pneumoniae, Streptococcus pyogenes.
